(19) United States
(12) Patent Application Publication
(10) Pub. No.: US 2021/0301038 A1
(43) Pub. Date: Sep. 30, 2021

(54) POLYSIALIC ACID AND DERIVATIVES THEREOF, PHARMACEUTICAL COMPOSITION AND METHOD OF PRODUCING POLYSIALIC ACID

(71) Applicants: Medizinische Hochschule Hannover (MHH), Hannover (DE); Deutsches Zentrum für Neurodegenerative Erkrankungen E.V. (DZNE), Bonn (DE)

(72) Inventors: Alexander Dityatev, Wahlitz (DE); Hristo Varbanov, Mainz (DE); Shaobo Jia, Magdeburg (DE); Rita Gerardy-Schahn, Hiddenhausen (DE); Evgeni Ponimaskin, Hannover (DE); Hauke Thiesler, Hannover (DE); Herbert Hildebrandt, Hannover (DE); Timm Fiebig, Hannover (DE)

(21) Appl. No.: 17/263,595

(22) PCT Filed: Jul. 31, 2019
(86) PCT No.: PCT/EP2019/070576

§ 371 Date: Jan. 27, 2021

(30) Foreign Application Priority Data
Jul. 31, 2018 (EP) 18186475.2

(51) Int. Cl. C08B 37/00; A61K 9/00; A61P 25/28

(52) U.S. Cl. CPC C08B 37/0006; A61P 25/28; A61K 9/0043

(57) ABSTRACT

The present invention relates to a polysialic acid according to the general formula (1) as given as follows and derivatives thereof: (α(2→8)Neu5Ac)ₙ, with n being an integer in the range from 6 to 13, for use in the prevention or treatment of neurological and neuropsychiatric disorders. The present invention also relates to a pharmaceutical composition comprising as an active ingredient said polysialic acid and/or derivatives and/or pharmaceutically acceptable salts thereof. Furthermore, the present invention relates to a method of producing said polysialic acid and/or pharmaceutically acceptable salts thereof.

### CROSS-REFERENCE TO RELATED APPLICATIONS

The present application claims the benefit of priority of EP Patent Application No. 18 186 475.2 filed 31 Jul. 2018, the content of which is hereby incorporated by reference in its entirety for all purposes.

### TECHNICAL FIELD OF THE INVENTION

The present invention relates to a polysialic acid according to the general formula (1) as given as follows and derivatives thereof: (α(2→8)Neu5Ac)ₙ with n being an integer in the range from 6 to 13, for use in the prevention or treatment of a neurological and neuropsychiatric disorder. The present invention also relates to a pharmaceutical composition comprising as an active ingredient said polysialic acid and/or derivatives and/or pharmaceutically acceptable salts thereof. Furthermore, the present invention relates to a method of producing said polysialic acid and/or derivatives and/or pharmaceutically acceptable salts thereof.

### BACKGROUND OF THE INVENTION

Dysregulation of polysialic acid (polySia) predominantly carried by the neural cell adhesion molecule (NCAM) as the major carrier protein of polysialic acid in vertebrate brains has been associated with several neuropsychiatric, neurological/neurodegenerative disorders, including schizophrenia, bipolar disorder, depression, and Alzheimer's disease (AD) (Brennaman and Maness, 2010). For example, in AD patients, polySia-NCAM expression in the entorhinal cortex is significantly decreased and negatively correlated with hyperphosphorylated Tau levels, one of the hallmarks of AD.

PolySia is a linear homopolymer that is composed of varying numbers of sialic acid residues (Hildebrandt et al., 2010; Schnaar et al., 2014). PolySia may though adopt a helical structure and can be identified with specific probes, such as antibodies and endo-N-acylneuraminidase (Endo-N). In the adult brain, polySia is typically found at very low levels. However, it is found in distinct regions where neural plasticity, remodeling of neural connections or neurogenesis is ongoing, such as the hippocampus, subventricular zone (SVZ), thalamus, prefrontal cortex and amygdala. In the mature hippocampus, polySia-NCAM is involved in regulation of N-methyl-D-aspartate receptor (NMDAR)-dependent synaptic plasticity.

The number of monomers in natural polysialic acids can reach 200. Most of the polySia chains on the mammalian glycoprotein neural cell adhesion molecule (NCAM) consist of a variable degree of sialic acid monomers. Extended polysialic acid chains have been observed on glycoproteins of human neuroblastoma.

However, there have been no causal studies investigating how the reduced polySia expression in the medial prefrontal cortex (mPFC) may affect cortical synaptic plasticity and cognitive functions in a neurological or neuropsychiatric disorder.

Therefore, there is a need in the art to provide new, alternative prevention and treatments of a neurological and neuropsychiatric disorder using short, defined-length fragments of polysialic acid.

Therefore, the objective of the present invention is to comply with this need.

The solution of the present invention is described in the following, exemplified in the appended examples, illustrated in the figures and reflected in the claims.

### SUMMARY OF THE INVENTION

The present inventors found that polysialic acids and/or derivatives thereof and/or pharmaceutically acceptable salts thereof according to the present invention may compensate for polysialic acid deficiency and be used for therapeutic targeting of extra synaptic NMDARs in a neurological and neuropsychiatric disorder, such as schizophrenia, a tauopathy, or amyloidosis. The efficiency of polysialic acids according to the present invention was clearly demonstrated for improvements of cortical synaptic and cognitive functions.

Therefore, the present invention deals with a polysialic acid according to the general formula (1) as given as follows and derivatives thereof:

(α(2→8)Neu5Ac)ₙ (1)

wherein Neu5Ac is N-acetylneuraminic acid, and
n is an integer in the range from 6 to 13,
for use in the prevention or treatment of a neurological and neuropsychiatric disorder,
wherein said derivatives of the polysialic acid are substituted with at least one sugar, acetyl group or acyl group at at least one monomer of the polysialic acid,
optionally linked to at least one nano-carrier,
and pharmaceutically acceptable salts thereof.

Additionally, the present invention may comprise the polysialic acid as mentioned above, wherein n is an integer in the range from 10 to 13.

The present invention may also envisage the polysialic acid as mentioned above, wherein the polysialic acid inhibits the activation of heterodimeric GluN1/GluN2B or heterotrimeric GluN1/GluN2A/GluN2B-containing NMDA receptors.

Further, the present invention may encompass the polysialic acid as mentioned above, wherein said derivatives are substituted with at least one sugar, acetyl group or acyl group at one monomer of the polysialic acid. Preferably, the present invention comprises the polysialic acid as mentioned above, wherein said derivatives are substituted with at least one sugar, acetyl group or acyl group at the one monomer at the non-reducing end of the polysialic acid.

The present invention may further envisage the polysialic acid as mentioned above, wherein the at least one sugar is glucose, N-acetylglucosamine, N-acetylgalactosamine, galactose, fucose, mannose or xylose.

Also comprised by the present invention may be a polysialic acid as mentioned above, wherein said derivatives thereof have the formula (2) as given as follows:

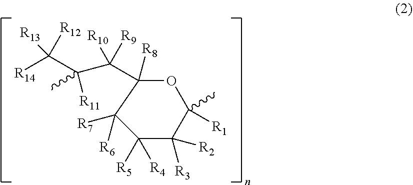

wherein at the one monomer at the non-reducing end of the polysialic acid:
i) R₁ is a carboxyl group;
ii) each of R₂, R₃, R₅, R₇, R₈, R₁₀, R₁₂, R₁₃ is independently from each other a hydrogen;
iii) R₄ is a hydroxyl group or an O-acetyl group;
iv) R₆ is NHCOCH₃;
v) R₉ is a hydroxyl group or an O-acetyl group;
vi) R₁₁ is a hydroxyl group or a hydrogen;
vii) R₁₄ is a hydroxyl group or an O-acetyl group.

Preferably, in said derivatives of said polysialic acid as defined in formula (2) at the one monomer at the non-reducing end of said polysialic acid R₄ is a hydroxyl group, R₆ is NHCOCH₃, R₉ is a hydroxyl group or an O-acetyl group, R₁₄ is an O-acetyl group. More preferably, in said embodiment wherein at the one monomer at the non-reducing end of said polysialic acid R₄ is a hydroxyl group, R₆ is NHCOCH₃, R₉ is a hydroxyl group or an O-acetyl group and R₁₄ is an O-acetyl group, n of formula (2) is an integer in the range from 10 to 13.

Additionally, the present invention may also encompass the polysialic acid as mentioned above, wherein the polysialic acid is chemically linked via its reducing end to a nano-carrier.

Also comprised by the present invention may be a polysialic acid as mentioned above, wherein the neurological and neuropsychiatric disorder is schizophrenia, tauopathy, bipolar disorder, depression, epilepsy, Huntington's disease, multiple sclerosis, amyotrophic lateral sclerosis or stroke. Additionally, the present invention may envisage the polysialic acid as mentioned above, wherein tauopathy comprises Alzheimer's disease, frontotemporal dementia (FTD), primary age-related tauopathy (PART), chronic traumatic encephalopathy, progressive supranuclear palsy (PSP), corticobasal degeneration, dementia with Lewy Bodies (DLB), frontotemporal dementia and parkinsonism linked to chromosome 17 (FTDP-17), argyrophilic grain disease (AGD), glial globular tauopathy, Pick's diseases, and Parkinson's disease.

The present invention also comprises a pharmaceutical composition comprising as an active ingredient the polysialic acid and/or derivatives and/or pharmaceutically acceptable salts thereof as mentioned above, optionally comprising a pharmaceutical acceptable carrier.

The present invention may also encompass the pharmaceutical composition as described above, wherein the pharmaceutically composition comprises a pharmaceutically acceptable carrier, wherein the pharmaceutical acceptable carrier is a solid pharmaceutical acceptable carrier, preferably lactose, terra alba, sucrose, talc, gelatin, agar, pectin, acacia, magnesium stearate or stearic acid, a liquid pharmaceutical acceptable carrier, preferably sugar syrup, peanut oil, olive oil or water, or a gaseous pharmaceutical acceptable carrier, preferably carbon dioxide or nitrogen.

Also comprised by the present invention may be the pharmaceutical composition as described above, wherein the pharmaceutical composition is administered intranasal, oral, dermal, rectal, parenteral, preferably intranasal. Additionally, the present invention may also encompass the pharmaceutical composition as described above, wherein parenteral administration comprises intravitreal injection, subcutaneous injection, intravenous injection, intraperitoneal injection, intramuscular injection, perfusion, infusion or topical administration.

Further, the present invention may envisage the pharmaceutical composition as described above for use in the prevention or treatment of a neurological and neuropsychiatric disorder. Additionally, the present invention may comprise the pharmaceutical composition as described above, wherein the neurological and neuropsychiatric disorder is schizophrenia, tauopathy, bipolar disorder, depression, epilepsy, Huntington's disease, multiple sclerosis, amyotrophic lateral sclerosis or stroke. The present invention may also encompass the pharmaceutical composition as described above, wherein tauopathy comprises Alzheimer's disease, frontotemporal dementia (FTD), primary age-related tauopathy (PART), chronic traumatic encephalopathy, progressive supranuclear palsy (PSP), corticobasal degeneration, dementia with Lewy Bodies (DLB), frontotemporal dementia and parkinsonism linked to chromosome 17 (FTDP-17), argyrophilic grain disease (AGD), glial globular tauopathy, Pick's diseases, and Parkinson's disease.

Finally, the present invention comprises a method of producing the polysialic acid and/or derivatives and/or pharmaceutically acceptable salts thereof as mentioned above, comprising:

a) dissolving colominic acid in acetic acid;
b) stopping the reaction of step a) with NaOH;
c) storing the mixture of step b);
d) separating oligo- and polymers by chromatography with a NaCl gradient;
e) obtaining the polysialic acid and/or derivatives and/or pharmaceutically acceptable salts thereof.

### BRIEF DESCRIPTION OF THE FIGURES

FIG. 1 shows the effects of polySia fragments on evoked NMDAR-EPSCs in endoNF-treated mPFC acute slices from C57BL/6J mice.

FIG. 1(A, C) shows a scheme of recordings (top left) and representative examples (top right) and time courses of normalized amplitudes (bottom) of evoked NMDAR-EPSCs (evoked by stimulation (S) at layers II/III and recorded (R) in layer V pyramidal neurons at the holding potential of −60 mV) during basal recording, followed by successive bath application of DP12 (NANA12) (1 μg/ml) (FIG. 1(A)) or DP1 (NANA1) (10 μg/ml, control compound) (FIG. 1(C)) and the GluN2B-subunit specific antagonist Ro 25-6981 (0.3 μM) in sham- and endoNF-treated slices. Note that DP12 inhibited NMDAR-EPSCs in endoNF-treated, but not in sham-treated slices. FIG. 1(B) shows a bar graph summarizing mean levels of NMDAR-EPSC inhibition after DP12 and DP12+Ro 25-6981 application (cells presented in FIG. 1(A)). Two-way ANOVA revealed significant effects of enzyme (sham/endoNF, P<0.001) and drug treatment (DP12/DP12+Ro 25-6981, P=0.005), and no enzyme x drug interaction (P=0.198). **P<0.01, Holm-Sidak post hoc test. FIG. 1(C) shows no effect of DP1, but a significant inhibition of NMDAR-EPSC amplitude by Ro 25-6981 (P<0.05, paired Student's t test) in endoNF-treated slices. FIG. 1(D) shows a bar graph summarizing effects of DP1 (NANA1), DP5 (NANA5), and DP12 (NANA12) on NMDAR-EPSC amplitude in endoNF-incubated slices (normalized to the mean Ro 25-6981-mediated inhibition of amplitude in endoNF slices). One-way ANOVA revealed a significant difference between treatment groups (P<0.001). *P<0.05, ***P<0.001, Holm-Sidak post hoc test. Numbers of cells recorded and mice used are presented in bars. Data are shown as mean±SEM values.

FIG. 2 shows the effects of the polySia mimetic tegaserod on synaptically evoked NMDAR-EPSCs in mPFC slices from C57BL/6 mice.

FIG. 2(A) shows a scheme of recordings (top left) and representative examples (top right, averages of 8-10 traces) of evoked NMDAR-EPSCs before (black) and after the application of tegaserod (0.1 μM, grey) recorded from layer V pyramidal cells in sham- and endoNF-incubated slices. A time course (bottom) of normalized NMDAR-EPSC amplitudes showing a strong inhibition of EPSCs by tegaserod in both sham- and endoNF-incubated slices, suggesting that it acts on both synaptic and extra synaptic NMDARs. NMDAR-EPSCs were recorded in whole-cell voltage-clamp mode at −60 mV and isolated pharmacologically in a conventional manner, but in the presence of the 5-HT₄ receptor antagonist RS 39604 (10 μM), to dissect the polySia-related effects of tegaserod. FIG. 2(B) shows a bar graph summarizing the mean levels of inhibition of NMDAR-EPSCs in sham- and endoNF-treated slices (P=0.125, Student's unpaired t test). Numbers of slices recorded and mice used are presented in the bars. Data are shown as mean±SEM values.

FIG. 3 shows the impact of endoNF, Ro 25-6981, DP12, and GlyT1 inhibitors on long-term potentiation (LTP) in polySia-depleted mPFC slices.

FIG. 3(A) shows the time courses of normalized mean slope of field excitatory postsynaptic potentials (fEPSPs), showing impaired theta-burst stimulation (TBS)-induced LTP in endoNF-compared with sham-treated slices. FIG. 3(B) shows normal LTP levels in endoNF-treated slices recorded in the presence of the GluN2B-selective antagonist Ro 25-6981 (0.3 μM, abbreviated as Ro25) or DP12 (1 μg/ml). FIG. 3(C) shows both glycine transporter type 1 inhibitors, sarcosine (0.75 mM) and SSR 504734 (3 μM), restored LTP magnitude in endoNF-treated slices to the level measured in sham-treated slices. Delivery of 5× theta-burst stimulation (TBS) is indicated by arrows. Insets show averages of 30 fEPSPs recorded during 10 min before TBS (black) and 50-60 min after TBS (gray), respectively, in each condition. The mean slope of fEPSPs recorded 10 min before TBS was taken as 100%. FIG. 3(D) shows a bar graph summarizing mean levels of LTP measured 50-60 min after TBS application in FIG. 3(A-C). Note that DP12 was tested at 1 μg/ml (1). DP5 was tested at 1 μg/ml (1) and 10 μg/ml (10). One-way ANOVA revealed a significant difference between groups (P<0.001). *P<0.05, P<0.01, ˜P<0.001, Holm-Sidak post hoc test. Numbers of slices recorded and mice used are presented in bars. Data are shown as mean±SEM values.

FIG. 4 shows the restoration of LTP in the mPFC of ST8SIA4-deficient mice by Ro 25-6981, DP12 and sarcosine.

FIG. 4(A) shows reduced levels of TBS-induced LTP in slices from St8sia4^{-/-} mice compared with St8sia4^{+/+} controls. FIGS. 4(B-D) show fully restored LTP levels in slices from St8sia4^{-/-} mice and unchanged LTP levels in St8sia4^{+/+} mice in the presence of Ro 25-6981 (0.3 μM) (FIG. 4(B)), polySia DP12 (1 μg/ml) (FIG. 4(C)), or sarcosine (0.75 mM) (FIG. 4(D)). FIG. 4(E) shows the summary of mean LTP levels in St8sia4 mice in FIG. 4(A-D). Two-way ANOVA revealed significant effects of genotype (P<0.03) and treatment (P<0.001), and a genotype x treatment interaction (P<0.001). ^{###}P<0.001, Holm-Sidak post hoc test. Bar graphs in FIG. 4(E) show LTP levels measured 50-60 min after TBS. Numbers of slices recorded and mice used are presented in bars in FIG. 4(E). Data are shown as mean+SEM values.

FIG. 5 shows novel object recognition and recency tests in St8sia4^{-/-} mice and after in vivo intra-mPFC injection of endoNF in C57BL/6 mice.

FIG. 5(A) shows the experimental design. In the novel object recognition test, sarcosine (600 mg/kg b.w., intraperitoneally), DP12/DP1 (1 mg/kg, intranasally), and DMB-DP12/DMB-DP2 (2 mg/kg, intranasally) were delivered 30 min before the encoding phase, and retention of memory was evaluated 2 hours later, in the retrieval phase. In the recency test, reagents were applied 30 min before the first of two encoding phases separated by 1 hour interval, and recency memory was then assessed 10 min later. In FIG. 5(B-D), upper panels show exploration times, while lower panels show discrimination ratio (%) in all tested groups and trials. FIG. 5(B) shows exploration time and object discrimination ratio before and after intra-mPFC endoNF injection in C57BL/6J mice (n=12). An arrow shows the timing of endoNF injection. There is impairment in novel object recognition at day 1 and 3 after endoNF intra-mPFC injection, but it can be rescued by sarcosine at day 2. Intranasal delivery of DP12, but not DP1, at day 5 could also restore cognitive function compared to a control compound (*P<0.05, **P<0.01, ***P<0.001, paired t test). Two-way repeated measures ANOVA for discrimination ratios on d-2, d-1, d1 and d2 (lower panel in FIG. 5(B)) revealed statistically significant effects of endoNF injection (P=0.015), sarcosine treatment (P=0.017), and a significant endoNF x sarcosine interaction (P=0.002). ^{###}P<0.001, Holm-Sidak post hoc test; ^{#}P<0.05, paired Student's t test. FIG. 5(C) show a rescue of St8sia4^{-/-} mice (n=10) performance in novel object recognition tasks by DMB-DP12. Untreated St8sia4^{-/-} mice (n=10) failed to discriminate between novel (N) and familiar (F) objects. Intranasal administration of DMB-DP12, but not of DMB-DP2, restored discrimination (*P<0.05, **P<0.01, ***P<0.001, paired t-test). Two-way repeated measures ANOVA for discrimination ratio (lower panel) revealed statistically significant effects of genotype (P=0.0049) and interaction between genotype and treatment (P=0.0216). ^{#}P<0.05, ^{##}P<0.01, Holm-Sidak post hoc test. FIG. 5(D) show a rescue of St8sia4^{-/-} mice (n=10) performance in the recent test by DMB-DP12. Untreated St8sia4^{-/-} mice (n=10) failed to discriminate between the most recent (R) and least recent (L) objects (D). Intranasal administration of DMB-DP12, but not of DMB-DP2, restored discrimination (*P<0.05, **P<0.01, ***P<0.001, paired t-test). Two-way repeated measures ANOVA for discrimination ratio (lower panel) revealed statistically significant effects of genotype (P=0.0429). ^{#}P<0.05, *P=0.0603, Holm-Sidak post hoc test. Data are shown as mean+SEM values.

FIG. 6 shows two-photon in vivo imaging of DP12 penetration in the mPFC.

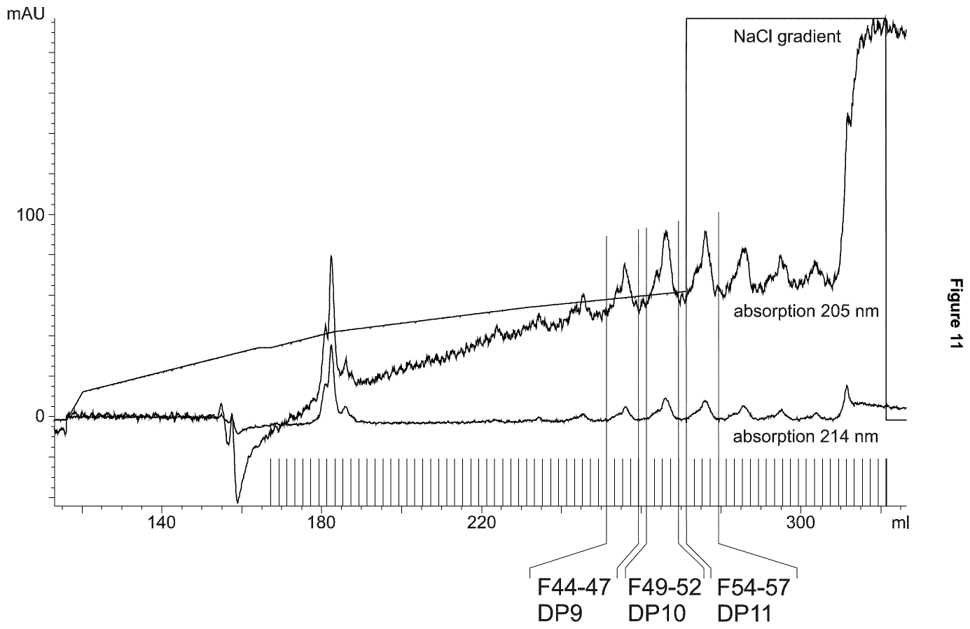

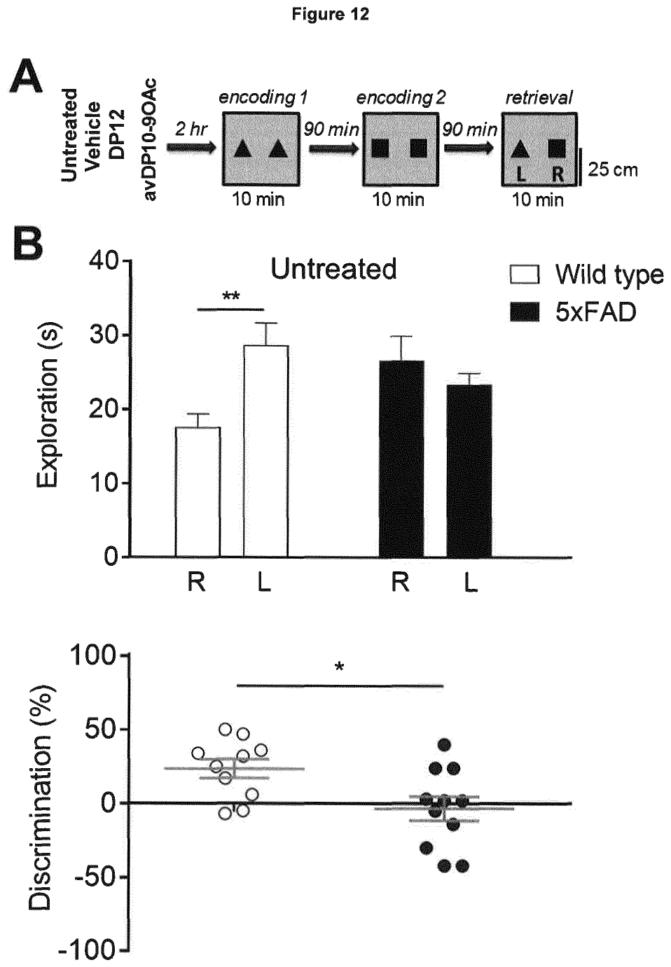

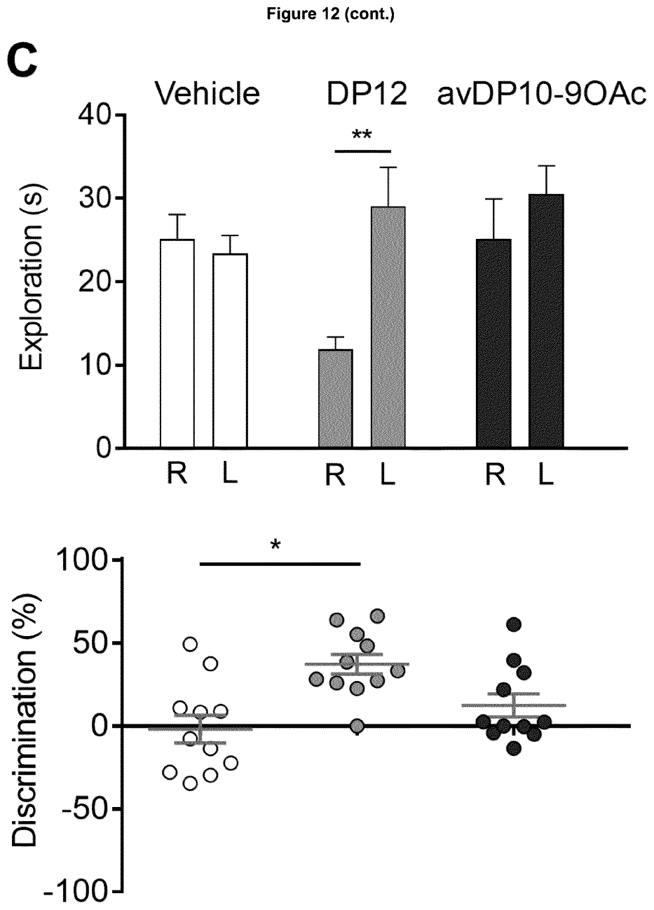

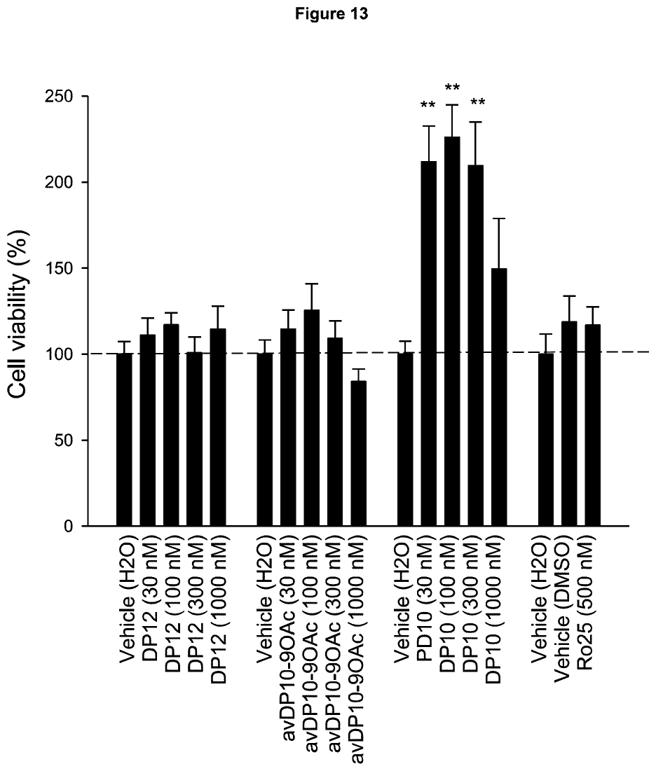

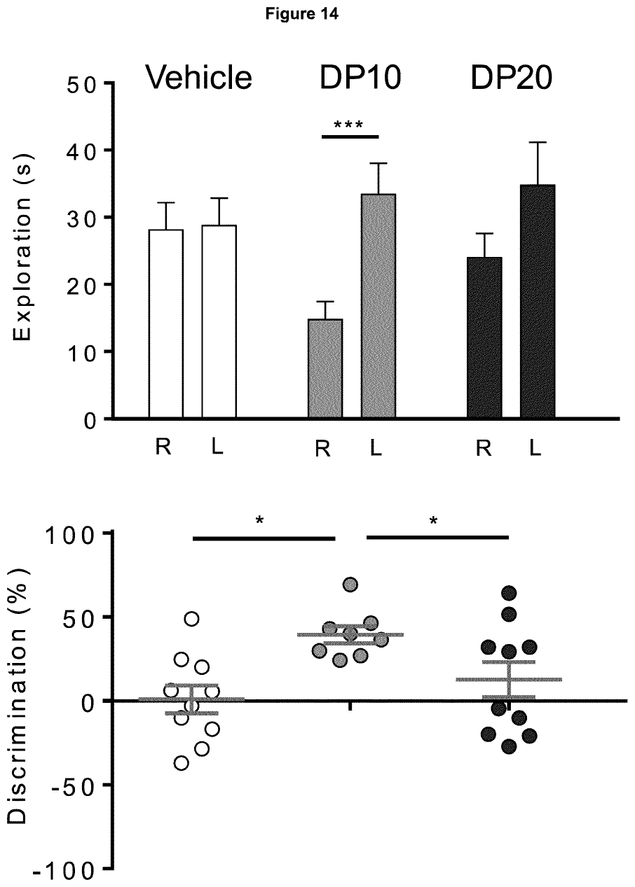

Time-lapse two-photon microscopy detection of 1,2-diamino-4,5-methylenedioxybenzene 2 HCl (DMB)-labelled DP2 and DP12 in the mPFC of adult Thy1-EGFP mice. FIG. 6(A) shows EGFP signal that was used to do imaging in the same position before (baseline, 0 h) as well as 0.5 h, 3 h, and 24 h after intranasal administration of drugs (10 mg/kg, 3 mice per group). FIG. 6(B) shows DMB signal in the same position where the EGFP imaging was done. Scale bar, 50 μm. FIG. 6(C) provides a statistical summary of imaging data from three mice. The DMB/EGFP ratio was used for quantification to compensate for unaccounted variability in laser intensity. Note the increase of fluorescent signal 0.5-3 hours after intranasal delivery of reagents. Data are shown as mean±SEM values.

FIG. 7 shows that DP12 rescues mPFC LTP and recency memory in a mouse model of tauopathy overexpressing mutated human Tau[R406W] protein in the mPFC.

FIG. 7(A) shows a scheme of experiment. FIG. 7(B) shows AAV-driven expression of GFP-Tau[R406W] and control GFP in the medial prefrontal cortex 1 month after injection of AAVs. Expression of GFP-Tau[R406W] leads to increased level of phosphorylated Tau (p-Tau). Scale bar, 100 μm. FIG. 7(C) shows that injection of Tau-GFP results in impaired mPFC LTP, which could be rescued by application of 0.3 μM DP12. Inserts above LTP profiles show fEPSPs recorded 10 min before and 50-60 min after induction of LTP. The bar plot depicts mean+SEM values of LTP 50-60 min after induction. *P<0.05, ***P<0.001, t-test. FIG. 7(D) shows a rescue of performance of AAV-GFP-Tau[R406W]-injected mice (n=10) in the recency test by DP12. Untreated AAV-GFP-Tau[R406W]-injected mice failed to discriminate the most recent (R) and least recent (L) objects, in contrast to AAV-GFP injected mice. Intranasal administration of DP12, but not of DP1, restored cognitive function in the recency test. *P<0.05, **P<0.01, ***P<0.001, paired Student's t-test. Two-way repeated measures ANOVA for discrimination ratio (lower panel in FIG. 7(D)) revealed statistically significant effects of Tau (P=0.0003), treatment (P=0.0205) and a significant Tau x treatment interaction (P=0.0001). ^{#}P<0.05, ^{###}P<0.001, Holm-Sidak post hoc test. Data are shown as mean+SEM values.

FIG. 8 shows that application of DP12 increased LTP in CA3-CA1 synapses in 5×FAD mice. FIG. 8(A) shows profiles of LTP induced in 5×FAD mice by theta-burst stimulation (at time 0) in control and 1 μg/ml (approx. 0.3 μM) DP12 bath-perfused slices. FIG. 8(B) shows mean±SEM of LTP levels 50-60 min after theta-burst stimulation. **P<0.01, t-test. Numbers of slices recorded and mice used are presented in bars.

FIG. 9 shows a column run after loading of 5 mg hydrolysed colominic. The positions of oligosialic acid with an n being 5 (DP5) and polysialic acid with an n being 26 (DP26) are indicated. The Figure shows isolation of single DPs from a DNAPac100 column. The elution gradient is monitored by the conductivity of the elution buffer (see legend). The recording at 280 nm demonstrates that the sample was free of protein contaminants. The elution profile of polysialic acid recorded at 214 nm demonstrates base line separation of single DPs. The positions of DP5 and DP26 are indicated by black arrows.

FIG. 10 shows that the oligosaccharide acceptor was successfully elongated with the supplied modified sialic acid. HPLC-AEC analysis with UV detection (214 nm) of the oligosaccharide products generated in the one-pot enzymatic elongation of DP9 with 9OAc-Sia. The elution time of the unmodified acceptor DP9 is indicated by a vertical line.

FIG. 11 shows the isolation and purification of 9OAc-modified oligosaccharides by FPLC-AEC. The fractions containing DP9-9OAc (F44-47), DP10-9OAc (F49-52) and DP11-9OAc (F54-57) are indicated. These fraction were pooled to obtain avDP10-9OAc.

FIG. 12(A) shows a modified scheme of recency test (increased difficulty) used in experiments with 5×FAD mice. FIG. 12(B) shows impaired performance of 5×FAD mice (n=11) in the recency test, while control wild-type mice (n=10) normally discriminated the most recent (R) and least recent (L) objects (**P<0.01, paired t-test). Discrimination ratio was significantly smaller in 5×FAD mice as compared to wild-types (*P<0.05, t-test). FIG. 12(C) shows that intranasal administration of DP12 restored cognitive function in the recency test. DP12-treated mice discriminated the most recent and least recent objects (**P<0.01, paired t-test). One-way repeated measures ANOVA for discrimination ratio (lower panel in FIG. 12(C)) revealed statistically significant difference between treatments (P=0.012). *P<0.05, post hoc Holm-Sidak test for comparison between vehicle and DP12. avDP10-9OAc had a tendency to improve object discrimination. Data are shown as means+SEMs.

FIG. 13 shows the effects of DP12, avDP10-9OAc, and DP10 on neural cell viability measured using the MTT assay. The cell viability represents means+SEMs of corrected optical densities in treated groups, which were expressed in % of mean value measured in control wells (treated with H₂O as vehicle) in the same plate. One-way ANOVA or Kruskal-Wallis test (in cases when the equal variance Brown-Forsythe test failed) were used for statistical analysis. No effects of DP12 and DP10-9OAc were detected by Kruskal-Wallis test (p=0.714, n=12 cultures per group) and one-way ANOVA (p=0.079, n=12 cultures per group), respectively. However, DP10 had a significant potentiating effect (p<0.001, Kruskal-Wallis test, 11 cultures per group) and Dunnett's post hoc test revealed a significant increase in cell viability in 30, 100 and 300 nM DP10-treated groups as compared to the vehicle group (p<0.01). Application of Ro25 had no effects on cell viability (one-way ANOVA, p=0.519, n=12 cultures per group).

FIG. 14 shows that intranasal administration of DP10, but not of vehicle or DP20 restored cognitive function in the recency test. Only DP10-treated mice discriminated the most recent and least recent objects (***P<0.001, paired t-test). One-way repeated measures ANOVA for discrimination ratio (lower panel) revealed statistically significant difference between treatments (P=0.0114), n=10, 5×FAD mice. *P<0.05, posthoc Holm-Sidak test. Data are shown as means+SEMs.

### DETAILED DESCRIPTION OF THE INVENTION

Although the present invention is described in detail below, it is to be understood that this invention is not limited to the particular methodologies, protocols and reagents described herein as these may vary. It is also to be understood that the terminology used herein is for the purpose of describing particular embodiments only, and is not intended to limit the scope of the present invention, which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and preferred embodiments described throughout the specification should not be construed to limit the present invention to only the explicitly described embodiments. This description should be understood to support and encompass embodiments, which combine the explicitly described embodiments with any number of the disclosed and/or preferred elements. Furthermore, any permutations and combinations of all elements described herein should be considered disclosed by the description of the present application unless the context indicates otherwise.

Throughout this specification and the claims which follow, unless the context requires otherwise, the word “comprise”, and variations such as “comprises” and “comprising”, will be understood to imply the inclusion of a stated member, integer or step or group of members, integers or steps, but not the exclusion of any other member, integer or step or group of members, integers or steps although in some embodiments such other member, integer or step or group of members, integers or steps may be excluded, i.e. the subject-matter consists in the inclusion of a stated member, integer or step or group of members, integers or steps. When used herein the term “comprising” can be substituted with the term “containing” or “including” or sometimes when used herein with the term “having”. When used herein “consisting of” excludes any element, step, or ingredient not specified.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in any suitable order unless otherwise indicated herein or otherwise clearly contradicted by context. The use of any and all examples, or exemplary language (e.g., “such as”), provided herein is intended merely to better illustrate the invention and does not pose a limitation on the scope of the invention otherwise claimed. No language in the specification should be construed as indicating any non-claimed element essential to the practice of the invention.

Unless otherwise indicated, the term “at least” preceding a series of elements is to be understood to refer to every element in the series. The term “at least one” refers to one or more such as two, three, four, five, six, seven, eight, nine, ten and more. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

When used herein “consisting of” excludes any element, step, or ingredient not specified in the claim element. When used herein, “consisting essentially of” does not exclude materials or steps that do not materially affect the basic and novel characteristics of the claim.

The term “including” means “including but not limited to”. “Including” and “including but not limited to” are used interchangeably.

The term “about” means plus or minus 10%, preferably plus or minus 5%, more preferably plus or minus 2%, most preferably plus or minus 1%.

It should be understood that this invention is not limited to the material and substances, etc., described herein and as such can vary.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent, the material incorporated by reference contradicts or is inconsistent with this specification, the specification will supersede any such material.

The content of all documents and patent documents cited herein is incorporated by reference in their entirety.

A better understanding of the present invention and of its advantages will be gained from the examples, offered for illustrative purposes only. The examples are not intended to limit the scope of the present invention in any way.

### Compound

The present invention relates to a polysialic acid according to the general formula (1) as given as follows: (α(2→8)Neu5Ac)ₙ

and pharmaceutically acceptable salts thereof,
wherein Neu5Ac is N-acetylneuraminic acid, and
n is an integer in the range from 6 to 13,
for use in the prevention or treatment of a neurological and neuropsychiatric disorder.

Further, the present invention relates to a polysialic acid according to the general formula (1) as given as follows: (α(2→8)Neu5Ac)ₙ

wherein Neu5Ac is N-acetylneuraminic acid, and
n is an integer in the range from 6 to 13,
for use in the prevention or treatment of a neurological and neuropsychiatric disorder, wherein said derivatives are substituted with at least one sugar, acetyl group, or acyl group at at least one monomer of the polysialic acid, optionally linked to at least one nano-carrier, and pharmaceutically acceptable salts thereof.

The term “polysialic acid” according to the present invention refers to homopolymers of sialic acid comprising 6-13 monomers, preferably 10, 11, 12 or 13 monomers. Additionally or alternatively, the term “polysialic acid” according to the present invention means sialic acid homopolymers with Neu5Ac residues at the position 5, wherein the homopolymers are linked via an α-2,8-linkage and which are recognized by the monoclonal antibody 735 (Frosch, M., Görgen, I., Boulnois, G. J., Timmis, K. N., Bitter-Suermann, D. (1985). Proc. Natl. Acad. Sci. USA, 82(4): 1194-8, PMID: 3919387). Based on a modified Farr assay (radioactive test system) according to Finne et al. (1983) and Chad (1980), a hexamer of an α-2,8-linked sialic acid with Neu5Ac at position 5 of each monomer, corresponding to a polysialic acid with an n being 6 (can be also called DP6) was demonstrated to be the minimal size needed to compete antibody 735 binding according to Hayrinen, J., Jennings, H., Raff, H. V., Rougon, G., Hanai, N., Gerardy-Schahn, R., Finne, J (1995) Journal Infect. Dis. 1995; 171(6):1481-90. This also means that the term “oligosialic acid” according to the present invention refers to homopolymers of sialic acid comprising 5 or less, e.g. 5, 4 or 3, monomers. The same may apply for the derivatives of the polysialic acid as defined herein and the respective pharmaceutically acceptable salts thereof.

In this context and throughout the present description, the term “monomer” refers to a molecule being able to undergo polymerization, thereby contributing constitutional units to the essential structure of a macromolecule. Large numbers of monomers combine to form polymers in a process called polymerization. Polymers are large molecules (macromolecules) composed of many repeating units (many monomers), where the number of monomers is in principle infinite. However, oligomers only consist of a few monomer units. In the specific context of the present invention, the term “monomer” may preferably mean one sialic acid molecule of the whole polysialic acid molecule with an integer n as described herein. The same may apply for the derivatives of the polysialic acid as defined herein and the respective pharmaceutically acceptable salts thereof.

The number of monomer units of the polysialic acid of the present invention may be depicted by “n” in the general formula (1). Having n=10, n=11, n=12, n=13 in formula (1) means having 10, 11, 12 or 13 monomers for said polysialic acid. Further, in the context of the present invention “DPn” refers to the polysialic acid as defined and used in the present invention with the respective n being an integer as described herein. This means, for example, that “DP12” refers to the polysialic acid as defined herein with n=12. The same may apply for the derivatives of the polysialic acid as defined herein and the respective pharmaceutically acceptable salts thereof.

Polysialic acid is an extended homopolymer of sialic acid. Sialic acid is a N- or O-substituted derivative of neuraminic acid. Polysialic acid/polySia (PSA) is found on glycoproteins, and is a component of the capsular polysaccharides of certain pathogenic bacteria. In bacteria, the sialic acid monomers of PSA can be linked by a 2.8 or a 2.9 linkage to form polysialic acid. In humans, the sialic acid monomer of PSA is linked by a 2.8 linkage and is acetylated at position 5.

The sialic acid monomer N-acetylated at position 5 usually is abbreviated Neu5Ac (N-acetylneuraminic acid). The monosaccharide Neu5Ac is denoted 5-acetamido-2,4-dihydroxy-6-(1,2,3-trihydroxypropyl)oxane-2-carboxylic acid according to the IUPAC nomenclature. The Neu5Ac monomers are linked by a (2→8) linkage to form polysialic acid molecules in the present invention. This may be achieved by using glycosidic bonds between the Neu5Ac monomer units of the polysialic acid.

At neutral pH, the α(2→8) linkages result in a highly flexible linear molecule, while at low pH the chemical structure of the polymer forms lactones, resulting in a more rigidified structure. The number of monomers in polysialic acid can reach 200. Most of the PSA chains on the mammalian glycoprotein neural cell adhesion molecule (NCAM) consist of a variable degree of sialic acid monomers. Extended polysialic acid chains have been observed on glycoproteins of human neuroblastoma.

In mammals, polysialic acid is added to the extracellular domain of NCAM by the two complementary polysialyltransferases ST8SIA2 and ST8SIA4. The neural cell adhesion molecule (NCAM) is a transmembrane glycoprotein that promotes cell-cell and cell-extracellular matrix adhesion. During development, NCAM regulates multiple processes, such as neurite outgrowth, neuronal migration, and synaptogenesis. The removal of polySia is performed by bacterial endosialidases, such as endoNF. In the mature hippocampus, polySia-NCAM is involved in N-methyl-D-aspartate receptor (NMDAR)-dependent synaptic plasticity.

A NMDA receptor (NMDAR) is a glutamate receptor and ion channel protein found in the membrans of nerve cells. It forms glutamate-gated ion channels that have central roles in neuronal communication and plasticity throughout the brain. It is activated when glutamate and glycine (or D-serine) bind to it, and when activated it allows positively charged ions, most importantly Ca²⁺, to flow through the cell membrane. Dysfunctions of NMDARs are involved in several central nervous system disorders, including stroke, chronic pain, dementia and schizophrenia. One hallmark of NMDARs is that their activity can be allosterically regulated by a variety of extracellular small ligands. The receptor is a heteromeric complex that interacts with multiple intracellular proteins by three different subunits: GluN1, GluN2 and GluN3. GluN1 has eight different isoforms (or also called slicing variants) generated by alternative splicing from a single gene. There are four different GluN2 subtypes (A-D) and late in the 20^{th} century, GluN3A and GluN3B subtypes have been reported. NMDARs form heterotetrameric complexes usually consisting of two GluN1 and two GluN2 subunits.

Because polysialic acid inhibits GluN2B-containing NMDARs only at the low micromolar glutamate concentrations characteristic of the extra synaptic space and because most extra synaptic NMDARs are thought to be GluN2B heterodimers, the inventors found it plausible that polysialic acid specifically inhibits extra synaptic NMDARs such as extra synaptic GluN1/2B receptors or extra synaptic GluN1/GluN2A/GluN2B receptors.

It was shown that acute enzymatic removal or genetic ablation of polysialic acid expression in the medial prefrontal cortex (mPFC), which both models the observed deficit of polysialic acid in schizophrenia, leads to increased transmission mediated by the GluN1/GluN2B subunits of NMDARs and impaired long-term potentiation (LTP). The latter could be fully rescued by application of short soluble polysialic acid fragments according to the general formula (1) as described elsewhere herein, which inhibited the activation of extra synaptic GluN1/GluN2B receptors and occluded the effects of 0.3 μM Ro 25-6981 (previously established specific treatment to antagonize GluN1/GluN2B receptors).

Thus, the present invention showed that said polysialic acid according to the general formula (1) as described elsewhere herein indeed inhibits the activation of heterodimeric GluN1/GluN2B and may inhibit heterotrimeric GluN1/GluN2A/GluN2B-containing NMDARs. The present invention thus demonstrated that polysialic acid according to the general formula (1) may be used for therapeutic targeting of extra synaptic NMDARs in a neurological and neuropsychiatric disorder, further being used in the prevention or treatment of a neurological and neuropsychiatric disorder.

As used herein and throughout the entire description, a neurological and neuropsychiatric disorder is schizophrenia, tauopathy, bipolar disorder, depression, epilepsy, Huntington's disease, multiple sclerosis, amyotrophic lateral sclerosis or stroke. In a preferred embodiment, a neurological and neuropsychiatric disorder is schizophrenia, tauopathy, or amyloidosis. In a more preferred embodiment, a neurological and neuropsychiatric disorder is schizophrenia or tauopathy. In an even more preferred embodiment, a neurological and neuropsychiatric disorder is a tauopathy.

In this context, the term “tauopathy” comprises Alzheimer's disease, frontotemporal dementia (FTD), primary age-related tauopathy (PART), chronic traumatic encephalopathy, progressive supranuclear palsy (PSP), corticobasal degeneration, dementia with Lewy Bodies (DLB), frontotemporal dementia and parkinsonism linked to chromosome 17 (FTDP-17), argyrophilic grain disease (AGD), glial globular tauopathy, Pick's diseases, Parkinson's disease. Preferably, a tauopathy is Alzheimer's disease.

In a preferred embodiment, the present invention comprises the polysialic acid according to the general formula (1) as defined elsewhere herein and pharmaceutical acceptable salts thereof for use in the prevention or treatment of schizophrenia or a tauopathy. In an even more preferred embodiment, the present invention comprises the polysialic acid according to the general formula (1) as defined elsewhere herein and pharmaceutical acceptable salts thereof for use in the prevention or treatment of a tauopathy, preferably of Alzheimer's disease.

As used herein and throughout the entire description, the term “pharmaceutically acceptable salt” refers to a salt that retains the desired biological activity of the respective compound and does not impart any undesired toxicological effects (see e.g., Berge, S. M., et al. (1977), J. Pharm. Sci., 66, 1-19). Physiologically acceptable salts of the compounds of the present invention are in particular salts with a non-toxic salt component and preferably are pharmaceutically utilizable salts. They can contain inorganic or organic salt components. Such salts can be formed, for example, from compounds of the present invention, which contain an acidic group, for example a carboxylic acid group (HO—CO—) or a sulfonic acid group (HO—S(O)₂—) and non-toxic, inorganic or organic bases. Suitable bases are, for example, alkali metal compounds or alkaline earth metal compounds, such as sodium hydroxide, potassium hydroxide, sodium carbonate or sodium hydrogencarbonate, or ammonia, organic amino compounds and quaternary ammonium hydroxides. Reactions of compounds of the present invention with bases for the preparation of the salts are in general carried out according to customary procedures in a solvent or diluent. On account of the physiological and chemical stability, advantageous salts of acidic groups are in many cases sodium, potassium, magnesium or calcium salts or ammonium salts, which can also carry one or more organic groups on the nitrogen atom. Compounds of the present invention, which contain a basic, i.e. protonatable, group, for example an amino group or another basic heterocycle, can be present in the form of their acid addition salts with physiologically acceptable acids, for example, a salt with hydrogen chloride, hydrogen bromide, phosphoric acid, sulfuric acid, acetic acid, benzoic acid, methanesulfonic acid, p-toluenesulfonic acid, which in general can be prepared from the compounds of the present invention by reaction with an acid in a solvent or diluent according to customary procedures. As usual, in particular, in the case of acid addition salts of a compound containing two or more basic groups, in an obtained salt, the ratio of the salt components can deviate upward or downward from the stoichiometric ratio, such as the molar ratio 1:1 or 1:2 in the case of the acid addition salt of a compound of the present invention containing one or two basic groups with a monovalent acid, and vary depending on the applied conditions. The present invention comprises also salts containing the components in a non-stoichiometric ratio, and an indication that an acid addition salt of a compound of the present invention contains an acid in equimolar amount, for example, also allows for a lower or higher amount of acid in the obtained salt, for example, about 0.8 or about 1.1 mol of acid per mol of compound of the present invention. If the compounds of the present invention simultaneously contain an acidic and a basic group in the molecule, the invention also includes internal salts (betaines, zwitterions) in addition to the salt forms mentioned.

As used herein and throughout the entire description, the term “pharmaceutically acceptable” may in particular mean approved by a regulatory agency or other generally recognized pharmacopoeia for use in animals, and more particularly in humans.

In a preferred embodiment, the “n” of formula (1) is an integer in the range from 10 to 13, such as 10, 11, 12, or 13. Having said that, the present invention comprises a polysialic acid having the following formula: (α(2→8)Neu5Ac)₁₀, (α(2→8)Neu5Ac)₁₁, (α(2→8)Neu5Ac)₁₂ or (α(2→8)Neu5Ac)₁₃ and derivatives thereof and pharmaceutical acceptable salts thereof. Having n=10, n=11, n=12, n=13 in formula (1) of the present invention means having 10, 11, 12 or 13 monomers of said polysialic acid having the general formula (1) as described above.

The polysialic acid according to the general formula (1) may optionally be linked to at least one nano-carrier. Thus, said polysialic acid of the present invention may be linked to at least one nano-carrier, preferably one nano-carrier. In this connection, the term “nano-carrier” refers to nanomaterial being used as a transport module for another substance (e.g. for the polysialic acid of the present invention). Commonly used nano-carriers may include, but are not limited to, lipid-based carriers, such as micelles and liposomes, polymers, carbon-based materials, polymeric nanoparticles, dendrimers, carbon nanotubes, and gold nanoparticles and other substances. Nano-carriers range from sizes of diameter 1-1000 nm. In the present invention a nano-carrier of <200 nm is preferably used. Nano-carriers are useful in the drug delivery process. They are able to deliver drugs to site-specific targets, allowing drugs to be delivered in certain organs or cells but not in others. Thus, this site-specificity is a major therapeutic benefit since it prevents drugs from being delivered to the wrong places. The nano-carrier being used in the present invention thus helps to deliver polysialic acid according to the general formula (1) as described elsewhere to its target/acting place (e.g. the brain), where it is able to inhibit extra synaptic NMDARs in a neurological and neuropsychiatric disorder.

The polysialic acid according to the general formula (1) may be chemically linked via its reducing end to a nano-carrier. In this context and as used throughout the present invention, the term “chemically linked” refers to a direct glycosidic linkage formed between the anomeric OH-group at position 2 of Neu5Ac and a second substrate or the use of a bifunctional linker that on one hand side reacts with the anomeric OH in position 2 of Neu5Ac and on the other hand reacts with a chemical group (usually a free amino-group) in the second substrate.

The term “reducing end of the polysialic acid” in this context and as used throughout the present invention refers to the Neu5Ac-residue of said polysialic acid according to the general formula (1), in which the anomeric center is not involved into the formation of an α-2,8-glycosidic linkage, even if it is conjugated through the hydroxyl-group at C2 (position 2). Biological polysialic acid is usually part of a glycan forming a posttranslational modification on proteins (primarily membrane anchored proteins). The conjugation to the glycan core structure (N-glycan or O-glycan) is through the reducing end. There may be several types of cell-type-specific polysialic acid membrane anchors, such as a membrane anchored protein molecule (e.g., N-glycosylated membrane anchored protein, O-glycosylated membrane anchored protein or a glycosylated, glycosylphosphoinositol (GPI)-membrane anchored protein). It may also be possible to link a nano-carrier to said reducing end of the polysialic acid according to the general formula (1) as indicated above. On the other hand, the term “non-reducing end of polysialic acid” in this context and as used through the present invention refers to the terminal sugar being α-2,8-glycosidically linked to the previous according to the general formula (1).

Additionally, also derivatives of said polysialic acid according to the general formula (1) as described elsewhere herein and pharmaceutical acceptable salts thereof may be used in the prevention or treatment of a neurological and neuropsychiatric disorder.

In this context and as used throughout the entire invention, the term “prevention” or “prevent/preventing” refers to a complete inhibition of the development of a neurological and neuropsychiatric disorder in a subject by applying the polysialic acid according to the general formula (1) as defined elsewhere herein and/or derivatives thereof and/or pharmaceutically acceptable salts thereof or a pharmaceutical composition comprising as an active ingredient the polysialic acid and/or derivatives and/or pharmaceutically acceptable salts thereof as described elsewhere herein.

The term “treatment” or “treat/treating” as used throughout the entire invention refers to halting the progression of a neurological and neuropsychiatric disorder in a subject, which has already been suffering from a neurological and neuropsychiatric disorder when the treatment with the polysialic acid according to the general formula (1) as defined elsewhere herein and/or derivatives thereof and/or pharmaceutically acceptable salts thereof or a pharmaceutical composition comprising as an active ingredient the polysialic acid and/or derivatives and/or pharmaceutically acceptable salts thereof as described elsewhere herein has been initiated.

A derivative in the context of the present invention refers to the polysialic acid according to the general formula (1) as described elsewhere herein being further substituted with at least one sugar, acetyl group or acyl group at at least one monomer of the polysialic acid.

In this context, the term “substituted at at least one monomer” means that a group (e.g. a hydrogen or hydroxyl group or the like) or more groups at (a) certain position(s) (e.g. at C1 and/or C2 and/or C3 and/or C4 and/or C5 and/or C6 and/or C7 and/or C8 and/or C9 of said polysialic acid) at at least one monomer of said polysialic acid of the present invention is being substituted/replaced with at least one sugar, acetyl group or acyl group, whereby said sugar, acetyl group, or acyl group replaces the group (e.g. a hydrogen or hydroxyl group or the like) being attached to said C-molecule (e.g. a hydrogen on C3 for example) or whereby said sugar, acetyl group or acyl group replaces more groups being attached at several, different C-molecules at at least one monomer, such as two, three, four, five, six, seven, eight, nine, ten, eleven, twelve, or thirteen monomers of the polysialic acid of the present invention.

In a preferred embodiment, said derivative of the present invention may be substituted with at least one sugar, acetyl group or acyl group at only one monomer of said polysialic acid according to the general formula (1). It is especially preferred, that said derivative of the polysialic acid according to formula (1) may be substituted with at least one sugar, acetyl group or acyl group at the one monomer at the non-reducing end of the polysialic acid.

In this context, the term “substituted at one monomer” means that a group (e.g. a hydrogen or hydroxyl group or the like) or more groups at (a) certain position(s) (e.g. at C1 and/or C2 and/or C3 and/or C4 and/or C5 and/or C6 and/or C7 and/or C8 and/or C9 of said polysialic acid) at one monomer of said polysialic acid of the present invention is being substituted/replaced with at least one sugar, acetyl group or acyl group, whereby said sugar, acetyl group or acyl group replaces the group (e.g. a hydrogen or hydroxyl group or the like) being attached to said C-molecule (e.g. a hydrogen on C3 for example) or whereby said sugar, acetyl group or acyl group replaces more groups being attached at several, different C-molecules at one monomer of the polysialic acid of the present invention.

When said derivatives of the polysialic acid according to formula (1), wherein n of formula (1) is 10, 11, 12 or 13, may be substituted with at least one sugar, acetyl group or acyl group at only one monomer of said polysialic acid, then a group (e.g. a hydrogen or hydroxyl group or the like) or more groups at (a) certain position(s) (e.g. at C1 and/or C2 and/or C3 and/or C4 and/or C5 and/or C6 and/or C7 and/or C8 and/or C9 of said polysialic acid) at one monomer of said polysialic acid having 10, 11, 12 or 13 sialic acid monomers is being substituted/replaced with at least one sugar, acetyl group or acyl group. It is especially preferred, that said derivative of the polysialic acid according to formula (1), wherein n of formula (1) is 10, 11, 12 or 13, may be substituted with at least one sugar, acetyl group or acyl group at the one monomer at the non-reducing end of the polysialic acid according to the general formula (1).

In another and even more preferred embodiment, said derivative of the present invention may be substituted with at least one sugar, acetyl group or acyl group at the one monomer at the non-reducing end of said polysialic acid according to the general formula (1).

In this context, the term “substituted at the one monomer at the non-reducing end” means that a group (e.g. a hydrogen or hydroxyl group or the like) or more groups at (a) certain position(s) (e.g. at C1 and/or C2 and/or C3 and/or C4 and/or C5 and/or C6 and/or C7 and/or C8 and/or C9 of said polysialic acid) at the one monomer at the non-reducing end of said polysialic acid of the present invention is being substituted/replaced with at least one sugar, acetyl group, or acyl group, whereby said sugar, acetyl group or acyl group replaces the group (e.g. a hydrogen or hydroxyl group or the like) being attached to said C-molecule (e.g. a hydrogen on C3 for example) or whereby said sugar, acetyl group or acyl group replaces more groups being attached at several, different C-molecules at the one monomer at the non-reducing end of the polysialic acid of the present invention.

When said derivatives of the polysialic acid according to formula (1), wherein n of formula (1) is 10, 11, 12 or 13, may be substituted with at least one sugar, acetyl group or acyl group at the one monomer at the non-reducing end of said polysialic acid, then a group (e.g. a hydrogen or hydroxyl group or the like) or more groups at (a) certain position(s) (e.g. at C1 and/or C2 and/or C3 and/or C4 and/or C5 and/or C6 and/or C7 and/or C8 and/or C9 of said polysialic acid) at the one monomer at the non-reducing end of said polysialic acid having 10, 11, 12 or 13 sialic acid monomers is being substituted/replaced with at least one sugar, acetyl group or acyl group.

In this context and as used throughout the entire description, the term “non-reducing end of polysialic acid” refers to the terminal sugar α-2,8-glycosidically linked to the previous according to the general formula (1). Polysialic acid chain growth occurs by the addition of sialic acid monomers to the non-reducing terminus of the growing chain. This may also be called tail growth.

The term “capping” in the context of the present invention means herein that polysialic acid according to the present invention is substituted only at its non-reducing end sugar. It is especially preferred that such a capping is an O-acetylation.

When the polysialic acid of the present invention is being substituted with at least one sugar at at least one monomer, at one monomer or at the one monomer at the non-reducing end of said polysialic acid of the present invention according to the general formula (1), said sugar molecule may be linked to said specific position at at least one monomer, at one monomer or at the one monomer at the non-reducing end of said polysialic acid of the present invention according to the general formula (1) via glycosidic linkage. Said linkage is a type of covalent bond that joins a carbohydrate (sugar) molecule to another group (e.g. the polysialic acid of the present invention), which may or may not be another carbohydrate. Glycosidic bonds are known to the person skilled in the art.

The term “sugar” according to the present invention is to be understood as meaning monosaccharides and disaccharides, which commonly are referred to as sugars. The at least one sugar may be glucose, N-acetylglucosamine, N-acetylgalactosamine, galactose, fucose, mannose or xylose. Advantageously, glucose, N-acetylglucosamine, N-acetylgalactosamine, galactose, fucose, mannose and xylose are essential sugars within the human body. The polysialic acid can comprise one terminal sugar molecule or be glycosidically bound to two or more sugars. Polysialic acid glycosidically linked to one or more sugar molecules can result in improved pharmacokinetics. The sugar molecule may be linked to the reducing end of said polysialic acid chain being linked via an α-(2,3)-linkage.

The term “glycosidically bound” according to the present invention is to be understood as meaning the polysialic acid that is bound to a further saccharide molecule, or other molecules capable of forming a glycosidic bond such as amino acids.

Thus, also comprised by the present invention may be derivatives of the polysialic acid of the present invention being substituted with at least one glucose, at least one N-acetylglucosamine, at least one N-acetylgalactosamine, at least one galactose, at least one fucose, at least one mannose or at least one xylose at at least one monomer, at one monomer or at the one monomer at the non-reducing end of said polysialic acid of the present invention according to the general formula (1).

Preferably, said derivatives of said polysialic acid according to the general formula (1) are substituted with at least one acetyl group at at least one monomer, at one monomer or at the one monomer at the non-reducing end of said polysialic acid of the present invention according to the general formula (1). More preferably, said derivatives of said polysialic acid according to the general formula (1) are substituted with at least one O-acetyl group at at least one monomer, at one monomer or at the one monomer at the non-reducing end of said polysialic acid of the present invention according to the general formula (1). Most preferably, said derivatives of said polysialic acid according to the general formula (1) are substituted with at least one O-acetyl group at the one monomer at the non-reducing end of said polysialic acid of the present invention according to the general formula (1).

The O-acetylation(s) of said derivative(s) of the present invention may be at position 4 (C4) and/or position 7 (C7) and/or position 9 (C9) at the one monomer at the non-reducing end of said polysialic acid according to the general formula (1). Preferably, the O-acetylation(s) of said derivative(s) of the present invention may be at position 7 (C7) and/or position 9 (C9) at the one monomer at the non-reducing end of the polysialic acid according to the general formula (1) as described herein.

More preferably, the O-acetylation of said derivative(s) of the present invention may be at position 9 (C9) at the one monomer at the non-reducing end of the polysialic acid according to the general formula (1) as described herein.

In a specially preferred embodiment, derivatives of said polysialic acid according to the general formula (1) being O-acetylated at position 9 at the one monomer at the non-reducing end and with a length of n being 10, refer to a polysialic acid according to the general formula (1), wherein n is 9, and an additional sialic acid according to the formula (α(2→8)Neu5Ac) being additionally O-acetylated at position 9. Thereby, the sialic acid according to the formula (α(2→8)Neu5Ac) being additionally O-acetylated at position 9 is at the non-reducing end of said polysialic acid according to the general formula (1), so that the complete polysialic acid has in total an n being 10.

In a specially preferred embodiment, derivatives of said polysialic acid according to the general formula (1) being O-acetylated at position 9 at the one monomer at the non-reducing end of the polysialic acid with a length of n being 11, refer to a polysialic acid according to the general formula (1), wherein n is 10, and an additional sialic acid according to the formula (α(2→8)Neu5Ac) being additionally O-acetylated at position 9. Thereby, the sialic acid according to the formula (α(2→8)Neu5Ac) being additionally O-acetylated at position 9 is at the non-reducing end of said polysialic acid according to the general formula (1), so that the complete polysialic acid has in total an n being 11.

In a specially preferred embodiment, derivatives of said polysialic acid according to the general formula (1) being O-acetylated at position 9 at the one monomer at the non-reducing end of the polysialic acid with a length of n being 12, refer to a polysialic acid according to the general formula (1), wherein n is 11 and an additional sialic acid according to the formula (α(2→8)Neu5Ac) being additionally O-acetylated at position 9. Thereby, the sialic acid according to the formula (α(2→8)Neu5Ac) being additionally O-acetylated at position 9 is at the non-reducing end of said polysialic acid according to the general formula (1), so that the complete polysialic acid has in total an n being 12.

In a specially preferred embodiment, derivatives of said polysialic acid according to the general formula (1) being O-acetylated at position 9 at the one monomer at the non-reducing end of the polysialic acid with a length of n being 13, refer to a polysialic acid according to the general formula (1), wherein n is 12 and an additional sialic acid according to the formula (α(2→8)Neu5Ac) being additionally O-acetylated at position 9. Thereby, the sialic acid according to the formula (α(2→8)Neu5Ac) being additionally O-acetylated at position 9 is at the non-reducing end of said polysialic acid according to the general formula (1), so that the complete polysialic acid has in total an n being 13.

The transfer of 9-O-acetylated sialic acid onto the non-reducing end of the polysialic acid according to the general formula (1), wherein n is an integer in the range from 10 to 13, such as 10, 11, 12 or 13, thereby producing the “derivatives” of the present invention, may be carried out with CMP activated 9-O-acetylated sialic acid and polysialic acid according to the general formula (1), wherein n is an integer in the range from 9 to 12, preferably in the molecular ratio 1.5:1 using the engineered bacterial derived NmB-polyST clone F116 (Keys et al., 2014). In particular, CMP-9-O-acetylated sialic acid was synthesized from 0.5 mM 5-acetamido-9-O-acetyl-3,5-dideoxy-D-glycero-galacto-non-2-ulosonic acid and 0.5 mM CTP using 0.5 μM of the CMP-sialic acid synthase (CMAS) from Neisseria meningitidis serogroup B. Further, 9-O-acetylated sialic acid was then transferred from CMP-9-O-acetylated sialic acid onto 0.1 mM of the acceptor oligosaccharide (e.g. a polysialic acid according to the general formula (1), wherein n is an integer of 9 using 0.5 μM of the distributive Neisseria meningitidis serogroup B polysialyltransferase (clone F116 (1)) as mentioned elsewhere herein.

The reaction buffer being used in the method described above concerning the synthesis of said derivatives of the present invention may comprise sodium phosphate, MgCl₂ and glycerol. In a preferred embodiment, the reaction buffer may comprise 50 mM phosphate, 10 mM MgCl₂ and 5% glycerol and a pH 8.0. Reactions may be incubated at 25° C. After 1 h (HPLC; DNAPac PA 100 column; Dionex, Sunnyvale, USA), modified polymer may be separated from non-modified material by preparative HPLC as it is described in paragraphs [00160]-[00161].

Said derivative(s) of the present invention may have the formula (2) as given as follows:

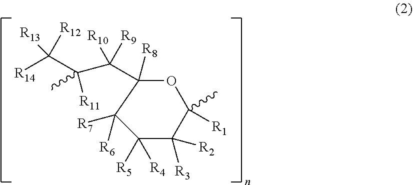

wherein at the one monomer at the non-reducing end of polysialic acid:
i) R₁ is a carboxyl group;
ii) each of R₂, R₃, R₅, R₇, R₈, R₁₀, R₁₂, R₁₃ is independently from each other a hydrogen;
iii) R₄ is a hydroxyl group or an O-acetyl group;
iv) R₆ is NHCOCH₃;
v) R₉ is a hydroxyl group or an O-acetyl group;
vi) R₁₁ is a hydroxyl group or hydrogen; and
vii) R₁₄ is a hydroxyl group or an O-acetyl group.

Said derivative(s) of the present invention may also have the formula (2) as given as follows:

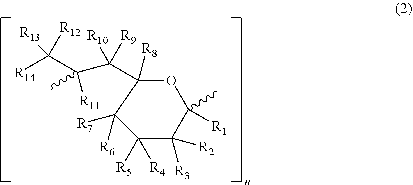

wherein at the one monomer at the non-reducing end of polysialic acid:
i) R₁ is a carboxyl group;
ii) each of R₂, R₃, R₅, R₇, R₈, R₁₀, R₁₂, R₁₃ is independently from each other a hydrogen;
iii) R₄ is a hydroxyl group or an O-acetyl group;
iv) R₆ is NHCOCH₃;
v) R₉ is a hydroxyl group or an O-acetyl group;
vi) R₁₁ is a hydroxyl group, and
vii) R₁₄ is a hydroxyl group or an O-acetyl group.

Said derivative(s) of the present invention may also have the formula (2) as given as follows:

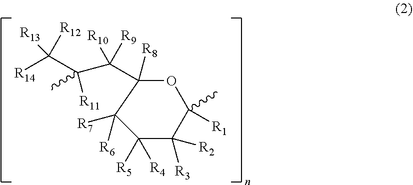

wherein at the one monomer at the non-reducing end of polysialic acid:
i) R₁ is a carboxyl group;
ii) each of R₂, R₃, R₅, R₇, R₈, R₁₀, R₁₂, R₁₃ is independently from each other a hydrogen;
iii) R₄ is a hydroxyl group or an O-acetyl group;
iv) R₆ is NHCOCH₃;
v) R₉ is a hydroxyl group or an O-acetyl group;
vi) R₁₁ is hydrogen, and
vii) R₁₄ is a hydroxyl group or an O-acetyl group.

In a preferred embodiment, n of formula (2) as described above is an integer in the range from 10 to 13, such as 10, 11, 12 or 13.

Thus, the present invention may comprise (a) derivative(s) according to formula (2) of the polysialic acid of the present invention, wherein at the one monomer at the non-reducing end:

i) R₁ is a carboxyl group;
ii) each of R₂, R₃, R₅, R₇, R₈, R₁₀, R₁₂, R₁₃ is independently from each other a hydrogen;
iii) R₄ is a hydroxyl group or an O-acetyl group;
iv) R₆ is NHCOCH₃;
v) R₉ is a hydroxyl group or an O-acetyl group;
vi) R₁₁ is a hydroxyl group or hydrogen;
vii) R₁₄ is a hydroxyl group or an O-acetyl group;
and wherein n is an integer in the range from 10 to 13, such as 10, 11, 12 or 13,
and pharmaceutically acceptable salts thereof,
for use in the prevention or treatment of a neurological and neuropsychiatric disorder.

The present invention may also comprise (a) derivative(s) according to formula (2) of the polysialic acid of the present invention, wherein at the one monomer at the non-reducing end:

i) R₁ is a carboxyl group;
ii) each of R₂, R₃, R₅, R₇, R₈, R₁₀, R₁₂, R₁₃ is independently from each other a hydrogen;
iii) R₄ is a hydroxyl group or an O-acetyl group;
iv) R₆ is NHCOCH₃;
v) R₉ is a hydroxyl group or an O-acetyl group;
vi) R₁₁ is a hydroxyl group;
vii) R₁₄ is a hydroxyl group or an O-acetyl group;
and wherein n is an integer in the range from 10 to 13, such as 10, 11, 12 or 13, and pharmaceutically acceptable salts thereof,
for use in the prevention or treatment of a neurological and neuropsychiatric disorder.

The present invention may also comprise (a) derivative(s) according to formula (2) of the polysialic acid of the present invention, wherein at the one monomer at the non-reducing end:

i) R₁ is a carboxyl group;
ii) each of R₂, R₃, R₅, R₇, R₈, R₁₀, R₁₂, R₁₃ is independently from each other a hydrogen;
iii) R₄ is a hydroxyl group or an O-acetyl group;
iv) R₆ is NHCOCH₃;
v) R₉ is a hydroxyl group or an O-acetyl group;
vi) R₁₁ is hydrogen;
vii) R₁₄ is a hydroxyl group or an O-acetyl group;
and wherein n is an integer in the range from 10 to 13, such as 10, 11, 12 or 13, and pharmaceutically acceptable salts thereof,
for use in the prevention or treatment of a neurological and neuropsychiatric disorder.

In another embodiment, the present invention may also comprise (a) derivative(s) having the formula (2) as described above, wherein at the one monomer at the non-reducing end of polysialic acid:

i) R₁ is a carboxyl group;
ii) each of R₂, R₃, R₅, R₇, R₈, R₁₀, R₁₂, R₁₃ is independently from each other a hydrogen;
iii) R₄ is an O-acetyl group;
iv) R₆ is NHCOCH₃;
v) R₉ is a hydroxyl group;
vi) R₁₁ is a hydroxyl group or a hydrogen;
vii) R₁₄ is a hydroxyl group;
and pharmaceutically acceptable salts thereof,
for use in the prevention or treatment of a neurological and neuropsychiatric disorder.

In another embodiment, the present invention may also comprise (a) derivative(s) having the formula (2) as described above, wherein at the one monomer at the non-reducing end of polysialic acid:

i) R₁ is a carboxyl group;
ii) each of R₂, R₃, R₅, R₇, R₈, R₁₀, R₁₂, R₁₃ is independently from each other a hydrogen;
iii) R₄ is an O-acetyl group;
iv) R₆ is NHCOCH₃;
v) R₉ is a hydroxyl group;
vi) R₁₁ is a hydroxyl group;
vii) R₁₄ is a hydroxyl group;
and pharmaceutically acceptable salts thereof,
for use in the prevention or treatment of a neurological and neuropsychiatric disorder.

In another embodiment, the present invention may also comprise (a) derivative(s) having the formula (2) as described above, wherein at the one monomer at the non-reducing end of polysialic acid:

i) R₁ is a carboxyl group;
ii) each of R₂, R₃, R₅, R₇, R₈, R₁₀, R₁₂, R₁₃ is independently from each other a hydrogen;
iii) R₄ is an O-acetyl group;
iv) R₆ is NHCOCH₃;
v) R₉ is a hydroxyl group;
vi) R₁₁ is a hydrogen;
vii) R₁₄ is a hydroxyl group;
and pharmaceutically acceptable salts thereof,
for use in the prevention or treatment of a neurological and neuropsychiatric disorder.

Also comprised by the present invention may be (a) derivative(s) having the formula (2) as described above, wherein at the one monomer at the non-reducing end of polysialic acid:

i) R₁ is a carboxyl group;
ii) each of R₂, R₃, R₅, R₇, R₈, R₁₀, R₁₂, R₁₃ is independently from each other a hydrogen;
iii) R₄ is an O-acetyl group;
iv) R₆ is NHCOCH₃;
v) R₉ is a hydroxyl group;
vi) R₁₁ is a hydroxyl group or a hydrogen;
vii) R₁₄ is a hydroxyl group;
and wherein n is an integer in the range from 10 to 13, such as 10, 11, 12 or 13, and pharmaceutically acceptable salts thereof,
for use in the prevention or treatment of a neurological and neuropsychiatric disorder.

Also comprised by the present invention may be (a) derivative(s) having the formula (2) as described above, wherein at the one monomer at the non-reducing end of polysialic acid:

i) R₁ is a carboxyl group;
ii) each of R₂, R₃, R₅, R₇, R₈, R₁₀, R₁₂, R₁₃ is independently from each other a hydrogen;
iii) R₄ is an O-acetyl group;
iv) R₆ is NHCOCH₃;
v) R₉ is a hydroxyl group;
vi) R₁₁ is a hydroxyl group;
vii) R₁₄ is a hydroxyl group;
and wherein n is an integer in the range from 10 to 13, such as 10, 11, 12 or 13, and pharmaceutically acceptable salts thereof,
for use in the prevention or treatment of a neurological and neuropsychiatric disorder.

Also comprised by the present invention may be (a) derivative(s) having the formula (2) as described above, wherein at the one monomer at the non-reducing end of polysialic acid:

i) R₁ is a carboxyl group;
ii) each of R₂, R₃, R₅, R₇, R₈, R₁₀, R₁₂, R₁₃ is independently from each other a hydrogen;
iii) R₄ is an O-acetyl group;
iv) R₆ is NHCOCH₃;
v) R₉ is a hydroxyl group;
vi) R₁₁ is a hydrogen;
vii) R₁₄ is a hydroxyl group;
and wherein n is an integer in the range from 10 to 13, such as 10, 11, 12 or 13, and pharmaceutically acceptable salts thereof,
for use in the prevention or treatment of a neurological and neuropsychiatric disorder.

In another embodiment, the present invention may also comprise (a) derivative(s) having the formula (2) as described above, wherein at the one monomer at the non-reducing end:

i) R₁ is a carboxyl group;
ii) each of R₂, R₃, R₅, R₇, R₈, R₁₀, R₁₂, R₁₃ is independently from each other a hydrogen;
iii) R₄ is a hydroxyl group;
iv) R₆ is NHCOCH₃;
v) R₉ is an O-acetyl group;
vi) R₁₁ is a hydroxyl group or a hydrogen;
vii) R₁₄ is an O-acetyl group;
and pharmaceutically acceptable salts thereof,
for use in the prevention or treatment of a neurological and neuropsychiatric disorder.

In another embodiment, the present invention may also comprise (a) derivative(s) having the formula (2) as described above, wherein at the one monomer at the non-reducing end:

i) R₁ is a carboxyl group;
ii) each of R₂, R₃, R₅, R₇, R₈, R₁₀, R₁₂, R₁₃ is independently from each other a hydrogen;
iii) R₄ is a hydroxyl group;
iv) R₆ is NHCOCH₃;
v) R₉ is an O-acetyl group;
vi) R₁₁ is a hydroxyl group;
vii) R₁₄ is an O-acetyl group;
and pharmaceutically acceptable salts thereof,
for use in the prevention or treatment of a neurological and neuropsychiatric disorder.

In another embodiment, the present invention may also comprise (a) derivative(s) having the formula (2) as described above, wherein at the one monomer at the non-reducing end:

i) R₁ is a carboxyl group;
ii) each of R₂, R₃, R₅, R₇, R₈, R₁₀, R₁₂, R₁₃ is independently from each other a hydrogen;
iii) R₄ is a hydroxyl group;
iv) R₆ is NHCOCH₃;
v) R₉ is an O-acetyl group;
vi) R₁₁ is a hydrogen;
vii) R₁₄ is an O-acetyl group;
and pharmaceutically acceptable salts thereof,
for use in the prevention or treatment of a neurological and neuropsychiatric disorder.

Also comprised by the present invention may be (a) derivative(s) having the formula (2) as described above, wherein at the one monomer at the non-reducing end:

i) R₁ is a carboxyl group;
ii) each of R₂, R₃, R₅, R₇, R₈, R₁₀, R₁₂, R₁₃ is independently from each other a hydrogen;
iii) R₄ is a hydroxyl group;
iv) R₆ is NHCOCH₃;
v) R₉ is an O-acetyl group;
vi) R₁₁ is a hydroxyl group or a hydrogen;
vii) R₁₄ is an O-acetyl group;
and wherein n is an integer in the range from 10 to 13, such as 10, 11, 12 or 13, and pharmaceutically acceptable salts thereof,
for use in the prevention or treatment of a neurological and neuropsychiatric disorder.

Also comprised by the present invention may be (a) derivative(s) having the formula (2) as described above, wherein at the one monomer at the non-reducing end:

i) R₁ is a carboxyl group;
ii) each of R₂, R₃, R₅, R₇, Ra, R₁₀, R₁₂, R₁₃ is independently from each other a hydrogen;
iii) R₄ is a hydroxyl group;
iv) R₆ is NHCOCH₃;
v) R₉ is an O-acetyl group;
vi) R₁₁ is a hydroxyl group;
vii) R₁₄ is an O-acetyl group;
and wherein n is an integer in the range from 10 to 13, such as 10, 11, 12 or 13, and pharmaceutically acceptable salts thereof,
for use in the prevention or treatment of a neurological and neuropsychiatric disorder.

Also comprised by the present invention may be (a) derivative(s) having the formula (2) as described above, wherein at the one monomer at the non-reducing end:

i) R₁ is a carboxyl group;
ii) each of R₂, R₃, R₅, R₇, R₈, R₁₀, R₁₂, R₁₃ is independently from each other a hydrogen;
iii) R₄ is a hydroxyl group;
iv) R₆ is NHCOCH₃;
v) R₉ is an O-acetyl group;
vi) R₁₁ is a hydrogen;
vii) R₁₄ is an O-acetyl group;
and wherein n is an integer in the range from 10 to 13, such as 10, 11, 12 or 13, and pharmaceutically acceptable salts thereof,
for use in the prevention or treatment of a neurological and neuropsychiatric disorder.

In a preferred embodiment, the present invention comprises (a) derivative(s) having the formula (2) as described above, wherein at the one monomer at the non-reducing end:

i) R₁ is a carboxyl group;
ii) each of R₂, R₃, R₅, R₇, R₈, R₁₀, R₁₂, R₁₃ is independently from each other a hydrogen;
iii) R₄ is a hydroxyl group;
iv) R₆ is NHCOCH₃;
v) R₉ is a hydroxyl group;
vi) R₁₁ is a hydroxyl group or a hydrogen;
vii) R₁₄ is an O-acetyl group;
and pharmaceutically acceptable salts thereof,
for use in the prevention or treatment of a neurological and neuropsychiatric disorder.

In a preferred embodiment, the present invention comprises (a) derivative(s) having the formula (2) as described above, wherein at the one monomer at the non-reducing end:

i) R₁ is a carboxyl group;
ii) each of R₂, R₃, R₅, R₇, Ra, R₁₀, R₁₂, R₁₃ is independently from each other a hydrogen;
iii) R₄ is a hydroxyl group;
iv) R₆ is NHCOCH₃;
v) R₉ is a hydroxyl group;
vi) R₁₁ is a hydroxyl group;
vii) R₁₄ is an O-acetyl group;
and pharmaceutically acceptable salts thereof,
for use in the prevention or treatment of a neurological and neuropsychiatric disorder.

In a preferred embodiment, the present invention comprises (a) derivative(s) having the formula (2) as described above, wherein at the one monomer at the non-reducing end:

i) R₁ is a carboxyl group;
ii) each of R₂, R₃, R₅, R₇, R₈, R₁₀, R₁₂, R₁₃ is independently from each other a hydrogen;
iii) R₄ is a hydroxyl group;
iv) R₆ is NHCOCH₃;
v) R₉ is a hydroxyl group;
vi) R₁₁ is a hydrogen;
vii) R₁₄ is an O-acetyl group;
and pharmaceutically acceptable salts thereof,
for use in the prevention or treatment of a neurological and neuropsychiatric disorder.

In an even more preferred embodiment, the present invention comprises (a) derivative(s) of the present invention having the formula (2) as described above, wherein at the one monomer at the non-reducing end:

i) R₁ is a carboxyl group;
ii) each of R₂, R₃, R₅, R₇, R₈, R₁₀, R₁₂, R₁₃ is independently from each other a hydrogen;
iii) R₄ is a hydroxyl group;
iv) R₆ is NHCOCH₃;
v) R₉ is a hydroxyl group;
vi) R₁₁ is a hydroxyl group or a hydrogen;
vii) R₁₄ is an O-acetyl group;
and wherein n is an integer in the range from 10 to 13, such as 10, 11, 12 or 13, and pharmaceutically acceptable salts thereof,
for use in the prevention or treatment of a neurological and neuropsychiatric disorder.

In an even more preferred embodiment, the present invention comprises (a) derivative(s) of the present invention having the formula (2) as described above, wherein at the one monomer at the non-reducing end:

i) R₁ is a carboxyl group;
ii) each of R₂, R₃, R₅, R₇, R₈, R₁₀, R₁₂, R₁₃ is independently from each other a hydrogen;
iii) R₄ is a hydroxyl group;
iv) R₆ is NHCOCH₃;
v) R₉ is a hydroxyl group;
vi) R₁₁ is a hydroxyl group;
vii) R₁₄ is an O-acetyl group;
and wherein n is an integer in the range from 10 to 13, such as 10, 11, 12 or 13, and pharmaceutically acceptable salts thereof,
for use in the prevention or treatment of a neurological and neuropsychiatric disorder.

In an even more preferred embodiment, the present invention comprises (a) derivative(s) of the present invention having the formula (2) as described above, wherein at the one monomer at the non-reducing end:

i) R₁ is a carboxyl group;
ii) each of R₂, R₃, R₅, R₇, R₈, R₁₀, R₁₂, R₁₃ is independently from each other a hydrogen;
iii) R₄ is a hydroxyl group;
iv) R₆ is NHCOCH₃;
v) R₉ is a hydroxyl group;
vi) R₁₁ is a hydrogen;
vii) R₁₄ is an O-acetyl group;
and wherein n is an integer in the range from 10 to 13, such as 10, 11, 12 or 13, and pharmaceutically acceptable salts thereof,
for use in the prevention or treatment of a neurological and neuropsychiatric disorder.

In a preferred embodiment, the present invention comprises (a) derivative(s) having the formula (2) as described above, wherein at the one monomer at the non-reducing end:

i) R₁ is a carboxyl group;
ii) each of R₂, R₃, R₅, R₇, R₈, R₁₀, R₁₂, R₁₃ is independently from each other a hydrogen;
iii) R₄ is an O-acetyl group;
iv) R₆ is NHCOCH₃;
v) R₉ is an O-acetyl group;
vi) R₁₁ is a hydroxyl group or hydrogen;
vii) R₁₄ is an O-acetyl group;
and pharmaceutically acceptable salts thereof,
for use in the prevention or treatment of a neurological and neuropsychiatric disorder.

In a preferred embodiment, the present invention comprises (a) derivative(s) having the formula (2) as described above, wherein at the one monomer at the non-reducing end:

i) R₁ is a carboxyl group;
ii) each of R₂, R₃, R₅, R₇, R₈, R₁₀, R₁₂, R₁₃ is independently from each other a hydrogen;
iii) R₄ is an O-acetyl group;
iv) R₆ is NHCOCH₃;
v) R₉ is an O-acetyl group;
vi) R₁₁ is a hydroxyl group;
vii) R₁₄ is an O-acetyl group;
and pharmaceutically acceptable salts thereof,
for use in the prevention or treatment of a neurological and neuropsychiatric disorder.

In a preferred embodiment, the present invention comprises (a) derivative(s) having the formula (2) as described above, wherein at the one monomer at the non-reducing end:

i) R₁ is a carboxyl group;
ii) each of R₂, R₃, R₅, R₇, R₈, R₁₀, R₁₂, R₁₃ is independently from each other a hydrogen;
iii) R₄ is an O-acetyl group;
iv) R₆ is NHCOCH₃;
v) R₉ is an O-acetyl group;
vi) R₁₁ is a hydrogen;
vii) R₁₄ is an O-acetyl group;
and pharmaceutically acceptable salts thereof,
for use in the prevention or treatment of a neurological and neuropsychiatric disorder.

In an even more preferred embodiment, the present invention comprises (a) derivative(s) of the present invention having the formula (2) as described above, wherein at the one monomer at the non-reducing end:

i) R₁ is a carboxyl group;
ii) each of R₂, R₃, R₅, R₇, R₈, R₁₀, R₁₂, R₁₃ is independently from each other a hydrogen;
iii) R₄ is an O-acetyl group;
iv) R₆ is NHCOCH₃;
v) R₉ is an O-acetyl group;
vi) R₁₁ is a hydroxyl group or a hydrogen;
vii) R₁₄ is an O-acetyl group;
and wherein n is an integer in the range from 10 to 13, such as 10, 11, 12 or 13, and pharmaceutically acceptable salts thereof,
for use in the prevention or treatment of a neurological and neuropsychiatric disorder.

In an even more preferred embodiment, the present invention comprises (a) derivative(s) of the present invention having the formula (2) as described above, wherein at the one monomer at the non-reducing end:

i) R₁ is a carboxyl group;
ii) each of R₂, R₃, R₅, R₇, R₈, R₁₀, R₁₂, R₁₃ is independently from each other a hydrogen;
iii) R₄ is an O-acetyl group;
iv) R₆ is NHCOCH₃;
v) R₉ is an O-acetyl group;
vi) R₁₁ is a hydroxyl group;
vii) R₁₄ is an O-acetyl group;
and wherein n is an integer in the range from 10 to 13, such as 10, 11, 12 or 13, and pharmaceutically acceptable salts thereof,
for use in the prevention or treatment of a neurological and neuropsychiatric disorder.

In an even more preferred embodiment, the present invention comprises (a) derivative(s) of the present invention having the formula (2) as described above, wherein at the one monomer at the non-reducing end:

i) R₁ is a carboxyl group;
ii) each of R₂, R₃, R₅, R₇, R₈, R₁₀, R₁₂, R₁₃ is independently from each other a hydrogen;
iii) R₄ is an O-acetyl group;
iv) R₆ is NHCOCH₃;
v) R₉ is an O-acetyl group;
vi) R₁₁ is a hydrogen;
vii) R₁₄ is an O-acetyl group;
and wherein n is an integer in the range from 10 to 13, such as 10, 11, 12 or 13, and pharmaceutically acceptable salts thereof,
for use in the prevention or treatment of a neurological and neuropsychiatric disorder.

In a further embodiment, derivatives of the present invention may also be optionally linked to a least one nano-carrier as it has already been described for the polysialic acid of the present invention. Thus, paragraphs [0072] to [0074] may be applicable to said derivative as described above as well.

A cell free production of polysialic acid according to the present invention is also possible and may use a Maltose-Binding-Protein (MBP)-tagged engineered bacterial enzyme (MBP-NmBpolyST-F116) as described in Keys et al. (2014). The protein may be solid phase fixed to an amylose matrix (GE Healthcare; no. 28918779) and 30 min incubated at 25° C. with CMP-Neu5Ac and a DP3 primer in 50 mM Tris-HCl, pH 8.0, 25 mM KCl, 20 mM MgCl₂, 5% glycerol. Because of the distributive nature of MBP-NmBpolyST-F116, the size of products can be simply controlled by the ratio DP3:CMP-Sia (e.g. the production of DP12 would request the ratio 1:9). Reactions may be carried out in 1-2 ml volumes and may be stopped by centrifugation. Supernatants may be further processed as described in FIG. 9.

### Pharmaceutical Composition

In a further aspect, the present invention provides a pharmaceutical composition comprising as an active ingredient the polysialic acid and/or derivatives and/or pharmaceutically acceptable salts thereof as defined elsewhere herein. Further, said pharmaceutical composition may comprise as an active ingredient the polysialic acid and/or derivatives and/or pharmaceutically acceptable salts thereof as defined elsewhere herein and a pharmaceutically acceptable carrier.

As used herein, “pharmaceutically acceptable carrier” includes any and all solvents, dispersion media, coatings, isotonic and absorption delaying agents, and the like that are physiologically compatible. The “pharmaceutically acceptable carrier” may be in the form of a solid, semisolid, liquid, gaseous or combinations thereof. Preferably, the carrier is suitable for intranasal, enteral (such as oral), dermal, rectal, topical or parenteral administration (such as intravenous, intramuscular, subcutaneous, spinal or epidermal administration (e.g., by injection or infusion)).

In one embodiment the pharmaceutically acceptable carrier is a solid pharmaceutical acceptable carrier, preferably lactose, terra alba, sucrose, talc, gelatin, agar, pectin, acacia, magnesium stearate or stearic acid. In another embodiment the pharmaceutically acceptable carrier is a liquid pharmaceutical acceptable carrier, preferably sugar syrup, peanut oil, olive oil or water. In another embodiment the pharmaceutically acceptable carrier is a gaseous pharmaceutical acceptable carrier, preferably carbon dioxide or nitrogen.

The polysialic acid according to the general formula (1) as described in present invention and/or derivatives thereof and/or pharmaceutical acceptable salts thereof are preferably administered to a patient in need thereof via a pharmaceutical composition. In one embodiment, the pharmaceutical composition comprises the polysialic acid according to the general formula (1) as described in the present invention and/or derivatives thereof and/or pharmaceutical acceptable salts thereof and one or more pharmaceutically acceptable excipient(s).

The term “excipient”, when used herein, is intended to indicate all substances in a pharmaceutical composition, which are not active ingredients (e.g., which are therapeutically inactive ingredients that do not exhibit any therapeutic effect in the amount/concentration used), such as, e.g., carriers, binders, lubricants, thickeners, surface active agents, preservatives, emulsifiers, buffers, flavoring agents, colorants, or antioxidants.

Thus, the pharmaceutical composition of the present invention may comprise the polysialic acid according to the general formula (1) as described in the present invention and/or derivatives thereof and/or pharmaceutical acceptable salts thereof, one or more pharmaceutically acceptable excipient(s) and a pharmaceutically acceptable carrier.

The compounds used in the present invention are generally applied in “pharmaceutically acceptable amounts” and in “pharmaceutically acceptable preparations”. Such compositions may contain salts, buffers, preserving agents, carriers and optionally other therapeutic agents.

The pharmaceutical composition may be administered to an individual intranasal, oral, dermal, rectal, topical or parenteral.

The expressions “parenteral administration” and “administered parenterally” as used herein mean modes of administration other than enteral administration (examples of enteral administration include oral and rectal administration), usually by injection, perfusion or infusion or topical application, and include, without limitation, intravitreal injection, subcutaneous injection, intravenous injection, intraperitoneal injection, intramuscular injection, as well as topical administration (e.g., epicutaneous or through mucous membranes (such as buccal, sublingual or vaginal)).

However, an intranasally administration of the pharmaceutical composition comprising as an active ingredient the polysialic acid according to the general formula (1) as described in the present invention and/or derivatives and/or pharmaceutically acceptable salts thereof as defined elsewhere herein is preferred.

The pharmaceutical composition may also comprise adjuvants such as preservatives, wetting agents, emulsifying agents, pH buffering agents, and dispersing agents.

Prevention of the presence of microorganisms may be ensured by sterilization procedures and/or by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents, which delay absorption such as aluminum monostearate and gelatin.

Regardless of the route of administration selected, the active compounds (the polysialic acid and/or derivatives and/or pharmaceutically acceptable salts thereof as defined elsewhere herein), which may be used in a suitable hydrated form, and/or the pharmaceutical compositions used according to the present invention, are formulated into pharmaceutically acceptable dosage forms by conventional methods known to those of skill in the art (cf., e.g., Remington, “The Science and Practice of Pharmacy” edited by Allen, Loyd V., Jr., 22^{nd} edition, Pharmaceutical Sciences, September 2012; Ansel et al., “Pharmaceutical Dosage Forms and Drug Delivery Systems”, 7^{th} edition, Lippincott Williams & Wilkins Publishers, 1999).

A pharmaceutical composition can be administered by a variety of methods known in the art. As will be appreciated by the skilled artisan, the route and/or mode of administration will vary depending upon the desired results. The pharmaceutical compositions containing one or more active compounds can be prepared with carriers that will protect the active compounds against rapid release, such as a controlled release formulation, including implants, transdermal patches and microencapsulated delivery systems. Biodegradable, biocompatible polymers can be used, such as ethylene vinyl acetate, polyanhydrides, polyglycolic acid, collagen, polyorthoesters and polylactic acid. Methods for the preparation of such compositions are generally known to those skilled in the art. (See, e.g. Sustained and Controlled Release Drug Delivery Systems, J. R. Robinson, ed., Marcel Dekker, Inc., New York, 1978.)

To administer the compound used in the present invention by certain routes of administration, it may be necessary to coat the compound with, or co-administer the compound with a material to prevent its inactivation.

Pharmaceutical compositions typically are sterile and stable under the conditions of manufacture and storage.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the individuals to be treated. Each unit contains a predetermined quantity of active compound calculated to produce the desired therapeutic effect in association with the required pharmaceutical carrier. The specification for the dosage unit forms used according to the present invention are dictated by and directly dependent on (a) the unique characteristics of the active compound and the particular therapeutic effect to be achieved, and (b) the limitations inherent in the art of compounding such an active compound for the treatment of sensitivity in individuals.

Generally, out of 100% (for the pharmaceutical formulations/compositions), the amount of active ingredient (in particular, the amount of the polysialic acid according to the general formula (1) or according to any of the general formula (2) as described in the present invention and/or derivatives thereof and/or pharmaceutical acceptable salts thereof used according to the present invention, will range from about 0.01% to about 99%, preferably from about 0.1% to about 70%, most preferably from about 1% to about 30%, wherein the reminder is preferably composed of the one or more pharmaceutically acceptable excipients.

The amount of active ingredient, e.g., the polysialic acid according to the general formula (1) or according to the general formula (2) as described in the present invention and/or derivatives thereof and/or pharmaceutical acceptable salts thereof used according to the present invention, in a unit dosage form and/or when administered to an individual or used in therapy, may range from about 0.1 mg to about 10000 mg (for example, from about 1 mg to about 5000 mg, such as from about 10 mg to about 2000 mg) per unit, administration or therapy. In certain embodiments, a suitable amount of such active ingredient may be calculated using the mass or body surface area of the individual, including amounts of between about 1 mg/kg and 500 mg/kg (for example between about 2 mg/kg and 250 mg/kg, such as between about 10 mg/kg and 100 mg/kg), or between about 1 mg/m² and about 4000 mg/m² (such as between about 10 mg/m² and about 3000 mg/m² or between about 100 mg/m² and about 2000 mg/m²).

Actual dosage levels of the active ingredients in the pharmaceutical compositions used according to the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start with doses of the compounds used according to the present invention at levels lower than that required in order to achieve the desired therapeutic effect and gradually increase the dosage until the desired effect is achieved. In general, a suitable daily dose of a composition used according to the present invention will be that amount of the compound, which is the lowest dose effective to produce a therapeutic effect. Such an effective dose will generally depend upon the factors described above. It is preferred that administration be oral, intravenous, intramuscular, intraperitoneal or subcutaneous, preferably administered proximal to the site of the target. If desired, the effective daily dose of a pharmaceutical composition may be administered as two, three, four, five, six or more sub-doses administered separately at appropriate intervals throughout the day, optionally, in unit dosage forms. While it is possible for a compound used according to the present invention to be administered alone, it is preferable to administer the compound as a pharmaceutical formulation/composition.

The pharmaceutical composition used according to the invention can also, if desired, be presented in a pack, or dispenser device, which can contain one or more unit dosage forms containing the active compound. The pack can for example comprise metal or plastic foil, such as blister pack. The pack or dispenser device can be accompanied with instruction for administration.

Also comprised by the present invention is the pharmaceutical acceptable composition comprising the polysialic acid according to the general formula (1) or according to the general formula (2) as defined elsewhere herein and/or derivatives thereof and/or pharmaceutical acceptable salts thereof for use in the prevention or treatment of a neurological and neuropsychiatric disorder. In a preferred embodiment, the present invention comprises the pharmaceutical acceptable composition comprising the polysialic acid according to the general formula (1) or according to the general formula (2) as defined elsewhere herein and/or derivatives thereof and/or pharmaceutical acceptable salts thereof for use in the prevention or treatment of schizophrenia or tauopathy. In an even more preferred embodiment, the present invention comprises the pharmaceutical acceptable composition comprising the polysialic acid according to the general formula (1) or according to the general formula (2) as defined elsewhere herein and/or derivatives thereof and/or pharmaceutical acceptable salts thereof for use in the prevention or treatment of a tauopathy, preferably of Alzheimer's disease.

### Method

The present invention also comprises a method of producing the polysialic acid and/or derivatives and/or pharmaceutically acceptable salts thereof as described elsewhere herein, comprising a) dissolving colominic acid in acetic acid; b) stopping the reaction of step a) with NaOH, c) storing the mixture of step b); d) separating oligo- and polymers by chromatography with a NaCl gradient; e) obtaining the polysialic acid and/or derivatives and/or pharmaceutically acceptable salts thereof.

In the present invention the term “manufacturing/manufacture” may be used interchangeably with the term “producing/produce” as used herein. In this context, the term “producing the polysialic acid and/or pharmaceutically acceptable salts thereof” as described herein means that the polysialic acid and/or pharmaceutically acceptable salts thereof may be manufactured by using the method of the present invention.

In this context and as used throught the description, the term “dissolving” means subjecting colominic acid to acidic hydrolysis in acetic acid (HAc). Preferably, at least 50 mg, 60 mg, 70 mg, 80 mg, 90 mg colominic acid is used for step a) of the method of the present invention. Even more preferably, 100 mg colominic acid is subjected to acidic hydrolysis in acetic acid (HAc) for step a) of the method of the present invention. Most preferably, 100 mg colominic acid is subjected to acidic hydrolysis in 100 mM acetic acid (HAc) for 10 min at 70° C. for step a) of the method of the present invention. Those conditions had been demonstrated to generate DP5-DP15 at high concentration.

The term “to stop/stopping the reaction” in step b) of the method of the present invention refers to halt a certain reaction, in this case, the reaction of acidic hydrolysis of step a), by adjusting the pH to a certain value, which is alkaline. In a preferred embodiment, to stop the reaction in step b) of the method of the present invention, the pH was adjusted to 10.5 by addition of NaOH. Even more preferably, 1 M NaOH was used to stop the reaction in step b) in the method of the present invention.

The mixture of step b) of the method of the present invention, which comprises the reacting components of step a) and NaOH was then stored at 4° C. for 48 h to minimize intramolecular lactonization. In this context and as used throughout the present invention, the term “to store/storing” means transferring (e.g. filling) said mixture of step b) in a tube, container vessel, or dish and then warehousing said tube, container, vessel or dish being filled with said mixture of step b) of the method of the present invention into a refrigerator for a certain amount of time.

The term “to separate/separating” as used herein refers to subjecting the mixture from step b) of the method of the present invention to exchange chromatography thereby separating oligosialic acids and polysialic acids of individual degrees of polymerization (DP). In particular, the mixture was subjected to anion exchange chromatography applying a DNAPac100 (22×250 mm column; ThermoScientific), preceded by a guard column (4×50 mm; ThermoScientific). Elution of oligo/polysialic acid fragments was detected at λ=214 nm. To visualize potential impurities the elution was in parallel monitored at λ=280 nm. In order to obtain baseline separation of single DPs, 5 mg aliquots were loaded and eluted with a quasi-curved gradient (FIG. 9). Using this system with loading buffer A (10 mM Tris-HCl pH 8.0) and elution buffer B (buffer A+1 M NaCl) at a flow rate of 3.5 ml/min, the homogenous isolation of DP2-DP26 was possible in the method of the present invention.

Optionally, said polysialic acid and/or pharmaceutically acceptable salts thereof as described elsewhere herein may also be formulated. Thus, the present invention may also comprise an additional step f) in the method of the present invention. “Formulating” the obtained polysialic acid and/or pharmaceutically acceptable salts thereof of the present invention means bringing the polysialic acid and/or pharmaceutically acceptable salts thereof to conditions, where the polysialic acid and/or pharmaceutically acceptable salts thereof can be stored for a longer time. Many different methods known in the art are available to stabilize the polysialic acid and/or pharmaceutically acceptable salts thereof as described in the present invention. By exchanging the buffer, in which the polysialic acid and/or pharmaceutically acceptable salts thereof is existent after separation, the polysialic acid and/or pharmaceutically acceptable salts thereof can be brought under conditions, where it is more stable. Different buffer substances and additives, such as sucrose, mild detergents, stabilizer and the like, known in the art can be used.

### EXAMPLES OF THE INVENTION

The following Examples illustrate the invention, but are not to be construed as limiting the scope of the invention.

### Materials and Methods

Animals:

All treatments and behavioral procedures were conducted in accordance with animal research ethics standards defined by German law and approved by the Ethical Committee on Animal Health and Care of the State of Saxony-Anhalt (42502-2-1159 DZNE, 42502-2-1343 DZNE, and 42502-2-1346 DZNE).

C57BL/6J, constitutively ST8SIA4-deficient (St8sia4^{-/-}; Eckhardt et al., 2000) and 5×FAD mice (Oakley et al., 2006) were bred at the animal facility of DZNE Magdeburg. St8sia4^{-/-} mice were backcrossed with C57BL/6J mice for >8 generations. 5×FAD mice were backcrossed with C57BL/6J mice for >10 generations. Mice heterozygous for St8sia4 were crossed to produce homozygous mutants and littermate wild-type controls.

For electrophysiological experiments in brain slices, we used adult 2- to 4-month-old C57BL/6J and St8sia4^{-/-} mice and their respective age-matched wild-type littermates from both sexes. For behavioral experiments, we used male 2- to 3-month-old C57BL/6J mice, 7- to 10-month-old St8sia4^{-/-} mice and their wild-type littermates, 12- to 14-month-old male 5×FAD mice for the comparison with age- and gender-matched wildtype controls and for evaluation of DP12 and avDP10-9OAc, and 10- to 16-month-old 5×FAD mice of both genders for evaluation of DP10 and DP20. All mice were kept in a reverse light-dark cycle (12:12 hours, light on at 9:00 μm) with food and water ad libitum, and they were tested during the dark phase of the cycle, when mice are active.

Slice Preparation:

Each mouse was quickly sacrificed by cervical dislocation. After decapitation, the brain was rapidly removed from the skull and placed in ice-cold artificial cerebrospinal fluid (aCSF) equilibrated with 95% O₂/5% CO₂, which was composed of the following (in mM): 250 sucrose, 24 NaHCO₃, 25 glucose, 2.5 KCl, 1.25 NaH₂PO₄, 2 CaCl₂, and 1.5 MgCl₂ (osmolarity 345-350 mOsm/kg, pH 7.4). Using a VT1200S vibratome (Leica, Nussloch, Germany), coronal slices containing the prelimbic and infralimbic cortices (1.9-1.3 mm anterior to bregma) were cut 350 μm or 400 μm thick, for patch clamp and extracellular recordings, respectively, in the same sucrose-containing aCSF. Then, the slices were transferred to a slice holding chamber filled with recording aCSF (osmolarity 300-305 mOsm/kg, pH 7.4) that contained 120 mM NaCl instead of sucrose (Eckhardt et al., 2000) and was continuously gassed with 95% O₂/5% CO₂.

For endoNF treatment in C57BL/6J mice, slices were first allowed to recover for 20 min at room temperature (22-24° C.) in recording aCSF and were then placed in a 24-well plate to be incubated in 2 ml of aCSF without (sham) or with addition of endoNF (10 μg/ml) at 35° C. for 2 hours. The 24-well plate was installed in a water bath (ED, Julabo, Seelbach, Germany). For field excitatory postsynaptic potential (fEPSP) recordings in knockout and transgenic mice, mPFC slices were incubated in recording aCSF for >2 hours at room temperature before recordings. For patch-clamp recordings in knockout mice, slices were incubated first at 35° C. for 20 min and then at room temperature for >30 min before recordings started. The aCSF was continuously gassed with 95% O₂/5% CO₂ during all incubation steps and recordings.

Whole-Cell Patch-Clamp Recordings:

For whole-cell patch-clamp recordings, layer V pyramidal neurons were visualized using a SliceScope Pro 6000 equipped with a 40× water-immersion objective and infrared differential interference contrast microscopy (Scientifica, Uckfield, UK). Pyramidal cells were identified by their triangular soma and long apical dendrites, and synaptically evoked excitatory postsynaptic currents (EPSCs) were recorded using patch pipettes (3-5 MQ) fabricated using borosilicate glass capillaries (wall thickness 0.315, length 100, outer diameter 1.5 mm, Hilgenberg) and a DMZ-Zeitz puller (Zeitz Instruments GmbH, Martinsried, Germany). The intracellular pipette solution contained the following (in mM): 140.7 Cs-methane-sulfonate, 5 NaCl, 1 MgCl₂, 0.2 EGTA, 10 HEPES, 3 ATP-Mg, 0.3 Na-GTP, and 3.1 QX-314 (osmolarity 295 mOsm/kg, adjusted to pH 7.2 with CsOH). EPSCs were evoked by electrical pulses (0.2 ms) at 0.033 Hz via a glass pipette placed in layer II/III of the mPFC (identically to fEPSPs). Mixed EPSCs showing a fast AMPAR-mediated component and a slow NMDAR-mediated component were recorded at a −60 mV holding potential in whole-cell voltage-clamp mode in normal aCSF. The stimulation intensity was adjusted to evoke mixed EPSCs with amplitudes of approximately 300 pA. The slow NMDAR-mediated component of EPSCs was pharmacologically isolated at −60 mV in modified aCSF containing low Mg²⁺ (0.1 mM) and high Ca²⁺ (3.2 mM, for adjustment of divalent cation concentrations), the selective antagonist of α-amino-3-hydroxy-5-methyl-4-isoxazolepropionic acid (AMPA)/kainate receptors NBQX (10 μM), and the selective antagonist of γ-aminobutyric acid (GABA)_{B} receptors CGP-55845 (2 μM) (Chen et al., 2003). The washout of Mg²⁺ required at least 25 min. Recordings at a holding potential near the resting membrane potential allowed us to obtain stable NMDAR-EPSC recordings from pyramidal cells lasting for 1-1.5 hours. Series resistance (Rₛ, range 10-25 MΩ) was compensated (20-30%) and monitored continuously. Neurons in which Rₛ changed by more than 20% throughout the experiment were not included in the analysis. In voltage-clamp mode, membrane potential was not corrected for liquid junction potential. Evoked EPSCs were recorded at room temperature unless stated otherwise in the Results section.

Extracellular fEPSP Recordings:

Prefrontal slices (400 μm) were transferred to a submerged-type recording MC membrane chamber (volume 2 ml; Scientific Systems Design Inc., Mississauga, Ontario, Canada) perfused with oxygenated recording ACSF at a flow rate of 4 ml/min and maintained at room temperature (22-25° C.), unless stated otherwise in the “Results”-section given below. Field EPSPs were evoked and recorded using thin-walled glass electrodes (borosilicate glass, wall thickness 0.188, length 100, and outer diameter 1.5 mm, Hilgenberg, Malsfeld, Germany) filled with recording aCSF in 400 μm-thick mPFC slices. In order to stimulate electrically the input fibers of pyramidal cells, the stimulation electrode (0.3-0.5 MΩ) was inserted into layers II/III of the prelimbic cortex. The recording electrode (2-2.5 MΩ) was placed in the vicinity of dendrites and cell bodies of pyramidal neurons in layer V (Huang et al., 2004).

Before LTP induction, basal synaptic transmission was recorded in each slice and the relationship between stimulus intensity and fEPSP slope was measured. Basal fEPSPs were evoked at 0.05 Hz for at least 10 min. The supramaximal slope was determined by gradually increasing the stimulation intensity until a population spike in the fEPSP became first visible. The stimulation intensity was adjusted to elicit a basal fEPSP with a slope of approximately 50% of the supramaximal value.

LTP was induced by application of five trains of theta-burst stimulation (TBS) with an inter-theta-train interval of 20 s (Brennaman et al., 2011). Each train consisted of 8 bursts delivered at 5 Hz. Each burst consisted of four pulses delivered at 100 Hz. Duration of pulses was 0.2 ms. For calculation of the LTP level, the mean fEPSP slope was measured between 50-60 min after TBS delivery, and it was then normalized to mean baseline fEPSP slope during 0-10 min before TBS. Stimulus artifacts in representative fEPSP examples in figures were blanked to facilitate the perception of fEPSPs recorded in the mPFC. Recording and analysis of LTP in St8Sia4^{-/-} mice was performed in a blind manner.

Drugs Used in In Vitro Electrophysiological Experiments.

The enzyme endosialidase NF (endoNF) (Stummeyer et al., 2005) and the short form of polysialic acid composed of 10 or twelve sialic acid residues (DP10 and DP12) were purified as described previously (Keys et al., 2014). Sialic acid (N-acetylneuraminic acid, DP1) and the pentamer of sialic acid sodium salt (DP5) were obtained from EY Laboratories (San Mateo, Calif., USA). DMB-labelled DP2 and DP12, and non-labelled avDP10-9OAc were synthesized in the laboratory of Rita Gerardy-Schahn (see sections Synthesis of DMB-DP2 and DMB-DP12 and Synthesis of avDP10-9OAc, respectively). The GluN2B-selective antagonist (αR,βS)-α-(4-hydroxyphenyl)-β-methyl-4-(phenylmethyl)-1-piperidinepropanol maleate (Ro 25-6981 maleate, abbreviated as Ro25), the subtype-unselective antagonist of NMDA receptors D-(−)-2-amino-5-phosphonopentanoic acid (AP5), the selective AMPA receptor antagonist 2,3-dioxo-6-nitro-1,2,3,4-tetrahydrobenzo[t]quinoxaline-7-sulfonamide disodium salt (NBQX), the antagonist of GABA_{A} and glycine receptors picrotoxin, the GABA_{B} receptor antagonist (2S)-3-[[(1 S)-1-(3,4-dichlorophenyl)ethyl]amino-2-hydroxypropyl](phenylmethyl) phosphinic acid hydrochloride (CGP-55845), and the selective 5-HT₄ receptor antagonist 1-[4-amino-5-chloro-2-(3,5-dimethoxyphenyl)methyloxy]-3-[1-[2-methylsulphonylamino]ethyl]piperidin-4-yl]propan-1-one hydrochloride (RS 39604 hydrochloride) were obtained from Tocris Bioscience (Bristol, UK). The selective antagonist of the glial glycine transporter 1 (GlyT1) N-methylglycine (sarcosine) and the partial agonist at the glycine modulatory site of NMDA receptors (R)-4-Amino-3-isoxazolidone, 4-Amino-3-isoxazolidinone (D-cycloserine, DCS) were purchased from Sigma Aldrich (Taufkirchen, Germany). The selective antagonist of GlyT1 (2-chloro-N—[(S)-phenyl[(2S)-piperidin-2-yl]methyl]-3-trifluoromethyl benzamide (SSR 504734) was purchased from Axon Medchem (Groningen, Netherlands). The 5-HT₄ receptor agonist (polySia mimetic) tegaserod maleate was purchased from Sequoia Research Products LTD (Pangbourne, UK). All antagonists and agonists were applied via the slice perfusion system. Sarcosine (0.75 mM) solution was freshly prepared prior to experiments in aCSF. For all other drugs, appropriate stock solutions in double-distilled water or DMSO were prepared according to the manufacturer's recommendations and stored at −20° C. The membrane-impermeable lidocaine and the antagonist of voltage-activated Na⁺ channels N-(2,6-Dimethylphenylcarbamoylmethyl)-triethylammonium chloride (QX-314 chloride) were purchased from Tocris Bioscience. Experiments for pharmacological restoration of LTP were performed in the presence of GlyT1 blockers, Ro 25-6981, DP5 and DP12, and drugs were added to the perfusion aCSF>20 min before the start of the fEPSP recordings.

Data Analysis and Statistics for In Vitro Electrophysiology:

The data were acquired using an EPC10 amplifier at a sampling rate of 10 kHz, low-pass filtered at 3 kHz, analyzed using PatchMaster software (Heka Elektronik, Lambrecht, Germany), SigmaPlot 12 or 13 (Systat Software Inc., San Jose, Calif., USA), or Igor Pro 6.37 (WaveMetrics, Lake Oswego, Oreg., USA), and presented using Adobe Illustrator CS5 (Adobe Systems Inc., San Jose, Calif., USA) or SigmaPlot.

For evoked EPSCs, the maximal peak amplitude and decay time were analyzed using the PatchMaster software. The decay time of EPSC was defined as the time interval beginning at 80% of the EPSC maximal amplitude (after the EPSC peak) and ending at 20% of that amplitude. The NMDAR/AMPAR ratio in each cell was determined by dividing the average peak amplitude of the pharmacologically isolated NMDAR current by the average peak amplitude of AMPAR-mediated current recorded in normal aCSF. EPSC averages were based on 8-10 consecutive traces in each condition. For tonic currents, the NMDAR-mediated holding current was measured during 5 consecutive 2 s intervals before and 3 min after AP5 application. The shift in holding current was assessed as the difference between mean amplitude values before and after the NMDAR antagonist application for each cell. For fEPSP recordings, cortical fEPSPs were low-pass filtered at 500 Hz, and the rising slope of the fEPSP was measured during its initial linear phase using PatchMaster. Recordings of fEPSPs in which the fiber volley amplitude changed by more than 15% were excluded from the analysis.

Statistical comparisons of two or more independent groups used unpaired Student's t tests or analysis of variance (ANOVA) followed by multiple comparisons of groups with post hoc Holm-Sidak t test (SigmaPlot software). If raw data in the groups were not normally distributed, the Mann-Whitney rank sum test was used (for two independent groups) or ANOVA analysis was performed on ranks (for three or more groups). Effects of pharmacological treatments within one group were tested using paired Student's t test. The level of statistical significance was set at 0.05. For electrophysiological recordings in brain slices, numbers of cells/slices recorded (n) and mice used (N) were shown in bar graphs as “n/N”. All data are shown as the mean±SEM.

Isolation of Oligosialic Acids and Polysialic Acids at Preparative Scale:

100 mg colominic acid were subjected to acidic hydrolysis in 100 mM acetic acid (HAc) for 10 min at 70° C., conditions that had been demonstrated to generate DP5-DP15 at high concentration. To stop the reaction, the pH was adjusted to 10.5 by addition of 1 M NaOH. The mixture was stored at 4° C. for 48 h to minimize intramolecular lactonization.

To separate oligosialic acids and polysialic acids of individual DP (degrees of polymerization), the mixture was subjected to anion exchange chromatography applying a DNAPac100 (22×250 mm column; ThermoScientific), preceded by a guard column (4×50 mm; ThermoScientific). Elution of oligo-/polysialic acids was detected at λ=214 nm. To visualize potential impurities, the elution was in parallel monitored at λ=280 nm. In order to obtain baseline separation of single DPs, 5 mg aliquots were loaded and eluted with a quasi-curved gradient as shown in FIG. 9. Using this system with loading buffer A (10 mM Tris-HCl, pH 8.0) and elution buffer B (buffer A+1 M NaCl) at a flow rate of 3.5 ml/min, the homogenous isolation of DP2-DP26 was possible.

Each single peak was selected. DP≥10 were desalted by use of an Amicon filtration unit with 3 kDa cut-off filter and DP≥2 to DP≤10 by use of a Sartorius device with 2 kDa cut-off filter. After desalting samples were stored at −20° C.

Synthesis of avDP10-9OAc:

For the generation of avDP10-9OAc, a one-pot reaction was performed, in which (i) CMP-9OAc-Sia was synthesized from 0.5 mM 5-acetamido-9-O-acetyl-3,5-dideoxy-D-glycero-galacto-non-2-ulosonic acid (9OAc-Sia, kindly provided by Prof. Mark von Itzstein, Institute for Glycomics, Griffith University, Gold Coast Campus, Australia; 70% purity, Sia:9OAc-Sia=30:70) and 0.5 mM CTP (Sigma) using 0.5 μM of the CMP-Sialic acid synthase (CMAS) from Neisseria meningitidis serogroup B and (ii) 9OAc-Sia was then transferred from CMP-9OAc-Sia onto 0.1 mM of the acceptor oligosaccharide DP9 (FIG. 10) using 0.5 μM of a distributive Neisseria meningitidis serogroup B polysialyltransferase (clone F116 (1)). The reaction was performed in a total volume of 2000 μl of reaction buffer containing 50 mM Tris pH 8.0, 25 mM KCl, 20 mM MgCl₂, 5% glycerol and 0.001 U/μl pyrophosphatase.

The reaction products were analyzed by HPLC-anion exchange chromatography (AEC) on a Prominence UFLC-XR (Shimadzu) equipped with a CarboPac PA-100 column (2×250 mm, Dionex). Samples were separated as described by Keys et al., 2012, with the minor adjustment that H₂O and 1 M NaCl were used as mobile phases M1 and M2, respectively. 50 μl of sample were loaded for the detection of polysaccharide at 214 nm. Products were separated using an elution gradient consisting of a −2 curved gradient from 0 to 30% M2 over 4 min followed by a linear gradient from 30 to 84% M2 over 33 min.

Preparative AEC was performed on an ÄKTA pure 25 (GE Healthcare) equipped with a DNAPac PA100 22×250 mm including a 22×50 guard column at a flow rate of 4.0 ml/min. 10 mM Tris pH 8.0 and 10 mM Tris pH 8.0+1 M NaCl were used as mobile phases M1 and M2, respectively. Samples were separated using a combination of ten consecutive linear gradients:

(i) 0-7% over 7 ml
(ii) 7-18% over 44 ml
(iii) 18-18% over 3 ml
(iv) 18-22% over 16 ml
(v) 22-28% over 48 ml
(vi) 28-32% over 40 ml
(vii) 32-100% over 0 ml
(viii) 100-100% over 50 ml
(ix) 100%-0% over 0 ml
(x) 0%-0% over 100 ml
Fraction collection was initiated in parallel with gradient iv. The fraction size was set to 2 ml. Fractions containing DPs of a specific size (DPs of known size had been used to equilibrate the column) were pooled as shown in FIG. 11 and each DP was desalted in six consecutive steps of a decimal series of dilution using Amicon centrifugal devices with 3 kDa MWCO. For the generation of avDP10-9OAc, fractions containing DP11, DP10 and DP9 were pooled (see F49-52 in FIG. 11) and freeze-dried.

Synthesis of DMB-DP2 and DMB-DP12:

DP fragments of specific sizes, including DP2 to DP13, were purified by anion exchange chromatography as described (Keys et al., 2014). The preparation of fluorescently labelled DPs followed the procedure described in (Keys et al., 2012) with some modifications. In brief, 10 mg of the α-2,8-linked sialic acid dimer (DP2) or 100 mg colominic acid (polySia chains produced by acidic hydrolysis of the E. coli K1 capsule polysaccharide exhibiting an average size of 40 sialic acid units) were dissolved in 20 mM DMB (1,2-diamino-4,5-methylenedioxybenzene.2HCl) with 1 M β-mercaptoethanol and 40 mM sodium dithionite to give a final concentration of 10 mg/ml. The solution was mixed with an equal volume of ice cold 40 mM trifluoric acid (TFA) and incubated for 48 h at 4° C. Then the reaction was stopped by addition of NaOH until the pH of the mixture was 10.0. To revert lactonisation of polySia (induced by TFA treatment), the mixture was kept at room temperature for 24 h. Separation of DMB-labeled oligo- and polymers was achieved by anion exchange chromatography on a MonoQ 10/100 column (Amersham Bioscience) with a NaCl gradient. The liquid phase (M1) for chromatography was 50 mM trishydroxymethylaminomethane (Tris)/HCl buffer, pH 8.0 and the gradient was built by adding M2 (M1 containing 1 M NaCl). The column was run with a flow rate of 4 ml/min. After sample loading, a washing step with 12 ml M1 was carried out, and DMB-labelled fragments were subsequently eluted using the following gradient: 0 to 8% M2 over 12 ml, 8 to 20% M2 over 56 ml, 20 to 45% M2 over 208 ml with a fraction size of 4 ml. The fractions containing DMP-DP12 were pooled and desalted using a desalting column. DMB-DP2 was purified using a DNA Pac PA 100 column (Thermo Fisher Scientific, 22×250 mm), with water (M3) and 4 M ammonium acetate (M4) as liquid phases. After sample loading, the column was washed with 5 ml M3, and the sample eluted with a linear gradient from 0 to 13% M4 over 15 ml and 13 to 21% over 15 ml. DMB-DP2 was then passaged over a reversed phase column to remove trace impurities and concentrate DMB-DP2, and an X Select™ CSH™ C18 column (Waters Cooperation, 5 μm, 4.6×250 mm) was used. Thereby, buffer M5 (5% methanol, 5% acetonitrile) served as loading buffer and liquid phase 1, while M6 (80% acetonitrile) was used as liquid phase 2. After sample loading, the column was rinsed with 5 ml M5 (flow rate of 1 ml/min) and eluted with 10 ml M6. The DMB-DP2 peak was collected and freeze dried.

Implantation of Cannulas:

Guide cannulas were fabricated using stainless steel tubing with outer/inner diameter Ø 0.711/0.483 mm (Science Products, Germany). Dummy cannulas were made from entomological pins, size 2, Ø 0.41 mm (FST, Germany). Chronic implantation of cannulas into the mPFC in mice was performed as described previously for intrahippocampal injections (Kochlamazashvili et al., 2012; Senkov et al., 2015; Senkov et al., 2006).

Injection of EndoNF In Vivo:

Acute injection of endoNF in vivo was done under a short isoflurane anesthesia. For intra-mPFC injection, we used a digitally controlled infusion system (UltraMicroPump, UMP3, and Micro4 Controller, WPI, USA) fed with a 10 μl-Hamilton syringe and NanoFil 135 μm (35 GA) beveled needle. The thin (135 μm) part of the needle went into the mouse brain during injection, whereas its thick part (430 μm) fit perfectly in the guide cannula (inside 483 μm). Injections were performed as follows: 1) a mouse was anesthetized with 1-3% isoflurane as during implantation; 2) it was put into the stereotaxic frame; 3) the dummy cannula was gently removed from the left guide cannula; 4) a NanoFil injecting needle was carefully placed inside of the guide cannula and advanced further down until reaching the marker (made with a permanent pen at the needle shank (430 μm) to visualize the point when the needle should reach the surface of the brain coming out of the guide cannula (equal to the length of guide cannula); 5) once the marker was reached, a slow (10 μm increment) step dial was used to deepen the needle into the brain; 6) endoNF (2 μg/μl) or vehicle was injected with the following settings: 250 nl per site as deep as 1.5 and 2.25 mm from the brain surface (total injection volume per hemisphere 500 nl, injection rate of 3 nl/s); 7) after injection was complete (˜5 min), the needle was left in the injection site for another 5 min and then gently removed; 8) the dummy cannula was then put back to its place; 9) the same procedure was performed for the right hemisphere. The whole injection process took approximately 20-25 min under isoflurane anesthesia, and the mice recovered rather quickly, starting to move in approximately 2-3 min after being returned to their home cages. Complete recovery of a mouse after such short anesthesia usually occurs within 3-5 min. After injection, mice were returned to their animal facility. Behavior experiments started on the day after injection at approximately the same time, so that we could perform cognitive tests 24 hours after injection.

The Preparation and Stereotaxic Injection of Adeno-Associated Viruses (AAVs):

AAVs were produced using the AAV helper free packaging systems AAV-DJ (Cell Biolabs, Inc.). In short, HEK293 cells were transfected with the pAAV-DJ and pHelper constructs along with the viral expression constructs (pAAV vectors) encoding either eGFP or eGFP-Tau[R406W] under the control of a synapsin promotor. After three days, viruses were harvested using freeze-thaw cycles. After lysis, non-viral DNA is digested with Benzonase, and lysate is filtered through a 0.22 μm filter, washed with PBS and concentrated using Amicon Ultra-15 Centrifugal Filter Units with a cut off of 50 kDa. Titers were determined to be >10¹⁰ GC/ml using quantitative Real-Time PCR with primers for the WPRE element in the viral genome.

For stereotaxic delivery of AAVs, mice were anesthetized in a closed chamber using isoflurane (1 ml for 30 s). The mice were then mounted in a stereotaxic frame (Narishige, Tokyo, Japan) and chronically anesthetized using 1.5% isoflurane in combination with O₂. During injections, viral vectors (AAV-eGFP and AAV-Tau-eGFP) were bilaterally injected (500 nl per site) in the prelimbic cortex (AP+1.7 mm; ML±0.30 mm; DV −2.2 mm; relative to Bregma, according to the mouse brain atlas (Franklin and Paxinos, 2007) at an injection rate of 3 nl/s followed by an additional 5 min to allow diffusion. The mice remained in their home cage for 4 weeks until the start of LTP recordings or recency test.

Novel Object Recognition Test:

A white open field arena (50×50×30 cm) was used in the novel object recognition task. All behavior was video recorded and analyzed automatically by software (ANY-maze, version 4.99, Stoelting Co., Wood Dale, Ill.). The test was performed using a standard protocol (Leger et al., 2013) that includes two phases: a) a familiarization/encoding phase: mice were placed for 10 min in the arena, during which they have to explore two identical objects positioned in the center of the arena; b) a test/retrieval phase: one familiar object and one novel object were placed in the center of the arena, and mice were allowed to explore for 10 min. In the same trial, objects were counterbalanced, and between trials, different sets of objects were used. The interval between the encoding and retrieval phases was 2 h.

Recency (Temporal Order) Memory Test:

The recency test comprised of two encoding phases followed by a retrieval phase (modified from Nelson et al., 2011). The interval between the encoding phases was 1 h, and the interval between the second encoding phase and the retrieval phase was 10 min, except for experiments in 5×FAD mice when both intervals were 90 min to make the task more difficult. In the first encoding phase, animals were placed into the open field arena and allowed to explore a pair of identical objects for 10 min. Then, a different pair of identical objects was presented in the second encoding phase (10 min total exploration time). During the retrieval phase, two different objects, one object from each encoding phase, were placed into the apparatus and animals were given 10 min to explore them.

Behavioral Measurements and Statistical Analysis:

Animals were randomly assigned to treatment groups using a randomized block design to counterbalance differences in age. The time of the day when tests were performed for different animals was also counterbalanced between treatment groups using a randomized block design. All measurements and statistical analyses were performed performed blindly, i.e. the tubes containing drugs/vehicle were coded. Exploration was defined as the animal approaching the object at a distance of less than 2 cm. The animal climbing onto the object with both hind- and forelimbs was not considered as exploration. In the same trial, objects were counterbalanced, and between trials, the placements of novel and familiar objects were changed. The objects used in the novel object recognition test were not employed in the recency test. Novelty detection was evaluated by calculating the discrimination ratio as follows: [novel object time−familiar object time]/[novel object time+familiar object time]×100%. In the recency test, the discrimination ratio was calculated as follows: [least recent object time−most recent object time]/[least object time+most recent object time]×100%. Unreliable measurements (about 10%), when animals spent in total ≤10 s near to both objects, were excluded from analysis.

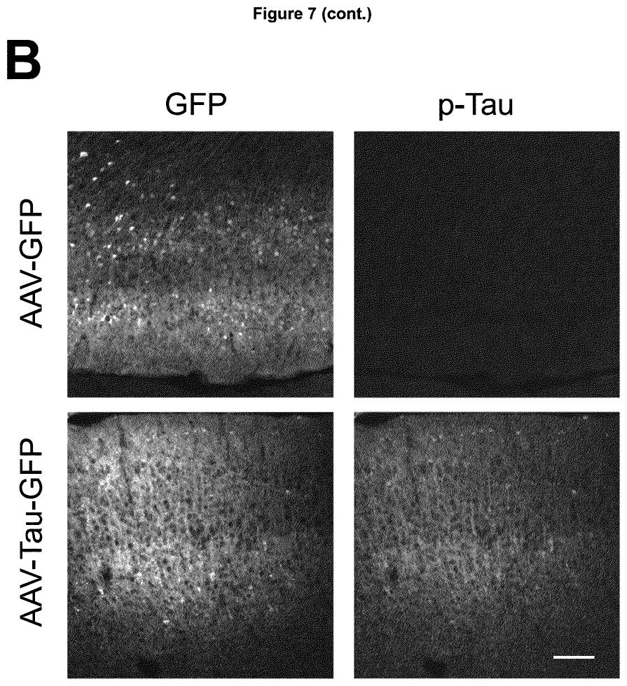

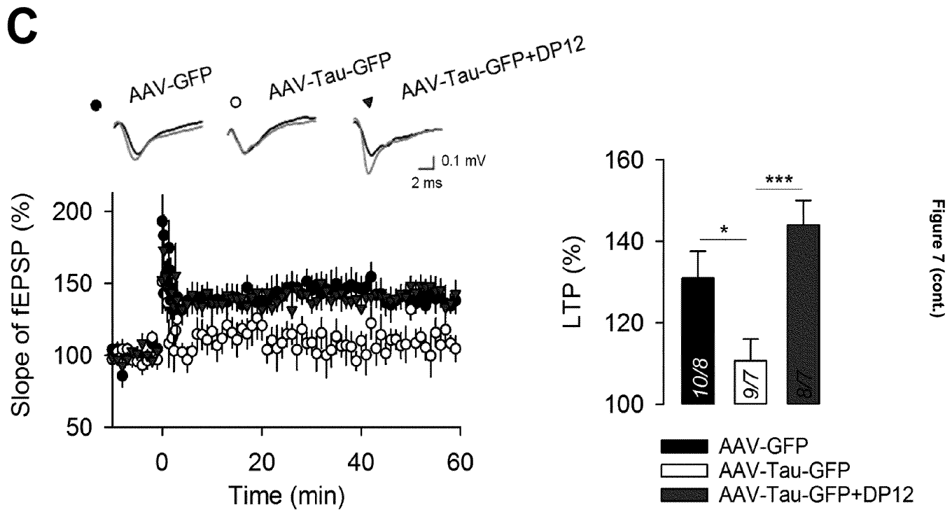

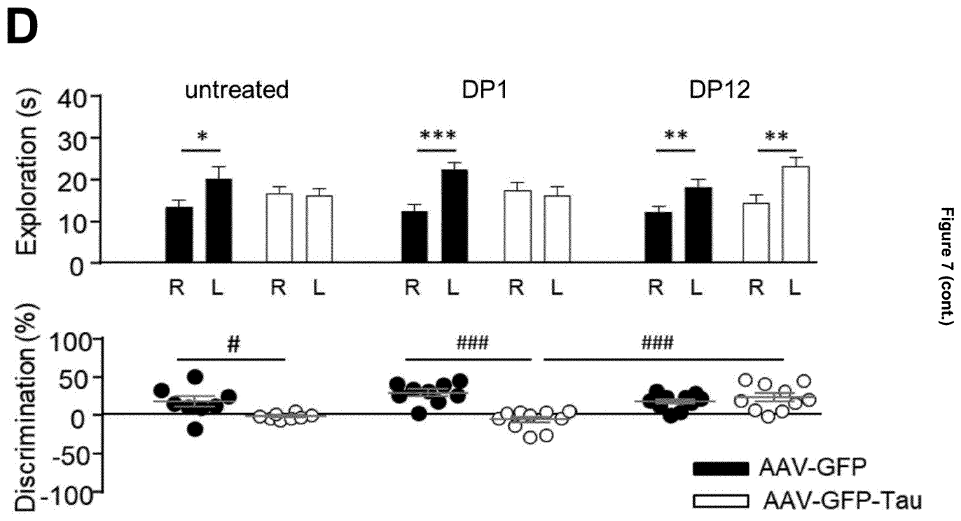

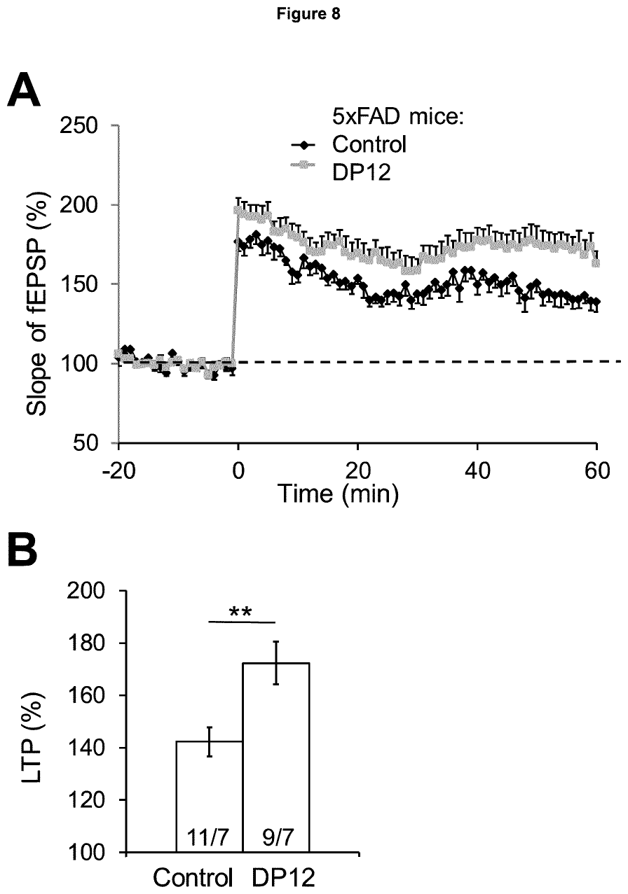

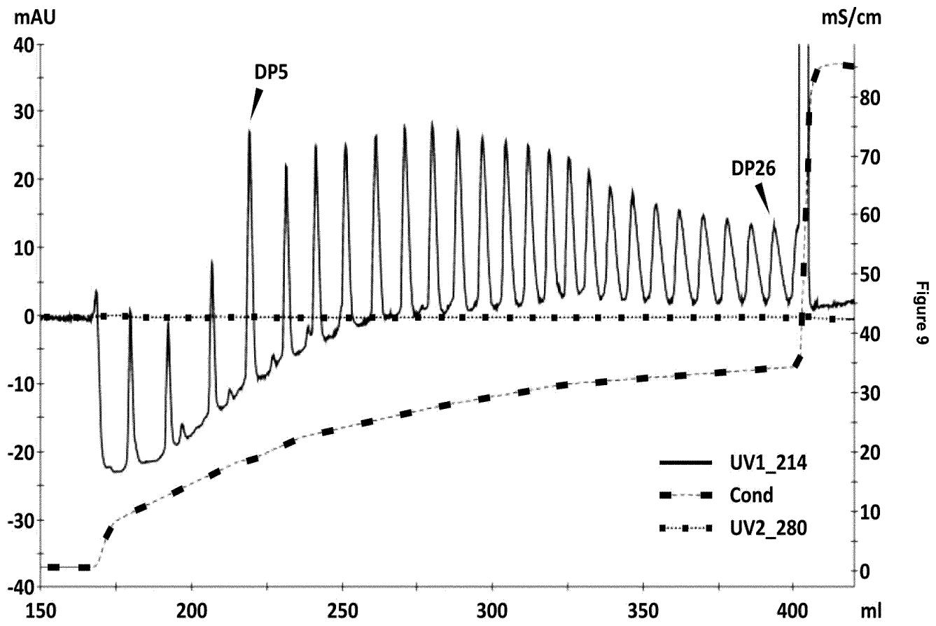

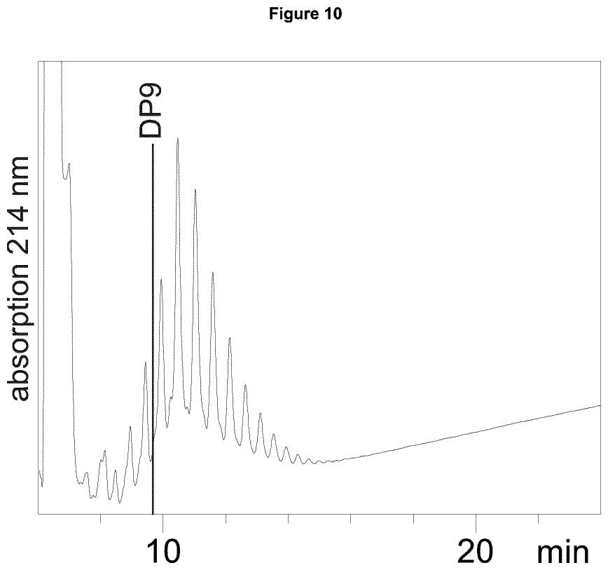

Results are expressed as the mean±SEM. SigmaPlot 12 or 13 and GraphPad 7 (La Jolla, Calif., USA) software were used for data analysis and presentation. Differences between the two genotypes undergoing two different treatments were assessed by two-way repetitive measures ANOVA followed by the Holm-Sidak post hoc test. Differences between times spent exploring familiar and novel objects were assessed using a paired Student's t test for each genotype in each treatment. For all comparisons, values of p<0.05 were considered significant. The Grubbs test was used to detect outliers (about 2%), which were excluded from analysis. Two-sided t test was used in all comparisons except for the recency test in FIG. 5(D, lower panel), when alternative hypothesis (based on the outcome of novel object recognition test, FIG. 5(C)) was that the discrimination ratio is lower for St8Sia4^{-/-} as compared to St8Sia4^{+/+} mice. In all experiments except for one shown in FIG. 14, only males were used for analysis. In that experiment, 6 males and 5 females were used. One female showed seizures and was excluded from analysis. As two-way ANOVA did not detect the effect of gender or interaction between gender and treatment, while detected the effect of treatment, the data for both genders were pulled together for further evaluation.

Immunohistochemistry for Phosphorylated Tau Protein

To investigate the expression of Tau in the mPFC of AAV-injected mice, coronal 400-μm-thick mPFC slices (two slices per mouse) were fixed in 4% paraformaldehyde in phosphate buffer (PB, 0.24 M, pH=7.2) for 1-2 hours at room temperature (RT) and cryoprotected using 30% sucrose in PB at 4° C. overnight. The sections were then mounted on a CM1950 cryostat (Leica Biosystems) using a tissue freezing medium (Jung, Leica Microsystems), and 35 μm-thick subsections were prepared (5-8 per a 400-μm-slice). After cryo-sectioning, slices were stored in cryo-protective solution (25% glycerin (Carl Roth, Karlsruhe, Germany), 25% ethylene glycol (Carl Roth) in 0.24 M PB) and stained “free-floating” using immunohistochemistry, as described below.

After washing in PBS (3×10 min, at RT), the slices were incubated in blocking solution (10% normal goat serum (NGS, Gibco®, Thermo Fisher Scientific, New Zealand origin) and 0.2% Triton X-100 (Sigma Aldrich) in PBS) at RT for 2 hours to reduce unspecific staining. For staining of phosphorylated Tau protein, we used a rabbit anti-Tau (phospho-T205) primary antibody (ab4841, Abcam, Cambridge, UK; dilution 1:500 in PB containing 5% NGS and 0.2% Triton X-100, at 4° C. for 48 hours). Sections were then washed again in PBS (3×10 min, at RT) and incubated in the secondary antibody (goat anti-rabbit Alexa 546 (Invitrogen), dilution 1:250 in PB containing 5% NGS, at RT for 3 hours). After washing in PB, the slices were stained for DAPI (dilution 1:1000, at RT for 10 min) and mounted on glass slides using Vectashield medium (Vector Laboratories, Burlingame, Calif., USA). Imaging of phospho-Tau staining was performed on a confocal laser-scanning microscope (LSM 700, Carl Zeiss, Germany) using 10× and 63× objectives, and the confocal images were analyzed using Zen software (Carl Zeiss, Jena, Germany).

Craniotomy and Implantation of mPFC Window:

Mice were first anesthetized with isoflurane in a chamber and were then placed into a stereotaxic apparatus on a 37° C. heating pad. The levels of isoflurane and oxygen were set to 1.5-2% 0.4 l/min, respectively, and the breathing rate was used for monitoring the depth of anesthesia. Ketoprofen (5 mg/kg) was injected to prevent inflammation and pain. To prevent corneal drying of the eyes during surgery, a lubricant ophthalmic ointment was applied. After cleaning the skin and shaving the fur, a cutaneous incision (length 8 mm) was made centrally over the frontal skull bone. Subsequently, a cranial window with a diameter of 5 mm was drilled using a dental micro motor. The center coordinates of the mPFC window were as follows: anterior-posterior (AP)+2.0 mm and medial-lateral (ML) 0 mm. After removing the bone, the brain pial surface was cleaned with 0.9% NaCl solution, and a 6.0-mm-diameter glass cranial window was implanted by applying a tiny layer of Roti Coll 1 superglue. The craniotomy was then sealed with dental cement. Finally, animals were placed into a recovery chamber and injected with ketoprofen (5 mg/kg) 24 h after the surgery.

Two-Photon In Vivo Imaging:

Mice were anesthetized intraperitoneally with ketamine (45 mg/kg body weight) and xylazine (18 mg/kg body weight) in 0.9% NaCl solution and then placed in a head fixation frame, while body temperature was maintained at 37° C. with a heating pad. Thirty minutes after injection, ˜1% isoflurane/O₂ gas mixture was employed to maintain anesthesia during in vivo microscopic imaging. Imaging was performed in the mPFC at the following stereotaxic coordinates: AP+1.9 mm and ML±0.5 mm. After imaging of mice under basal conditions, they were removed from the head fixation frame to apply intranasally DMB-labelled DP2 or DP12 in the same manner as described for behavioral experiments, but at concentration of 10 mg/kg, to increase the fluorescent signal. The imaging session was then continued for up to 3 h and repeated 24 h after intranasal application.

To visualize the penetration of DMB-labelled molecules in the mPFC, we have used Zeiss multiphoton microscope (LSM 7 MP) with a Ti:Sapphire laser tuned to 850 nm and 760 nm laser wavelength and water immersion objective (Zeiss, 20×) for transcranial imaging. To minimize photo toxicity, the average excitation laser power P was kept at a minimum for a sufficient signal-to-noise ratio. Fluorescence was detected using BP 420-480 and BP 500-550. We performed z-stack recording at 200-450 μm depth below the pial surface before (baseline) and at 0.5 h, 3 h and 24 h after application of DMB-labelled DP2 and DP12. Images were analyzed for mean intensity at a single plane within the mPFC cortical layer II at the respective time points, and 3 animals per group were used for analysis. Adult Thy1-EGFP mice (expressing the EGFP signal in a small fraction of neurons) were used for this analysis to facilitate repetitive findings the same imaging position.

Culturing of Neural Primary Cells:

Primary hippocampal cells were dissociated from embryonic mice on embryonic day 18. To prepare the primary neural cultures, pregnant C57/BL6J mice were sacrificed by cervical dislocation. Embryos were decapitated, and the hippocampi isolated from brains under sterile conditions and dissected. Enzymatic digestion by 0.25% trypsin/EDTA solution was followed by separation of the cells in serum-containing medium via trituration (10 passes, 1 ml pipette). The primary cells were seeded onto 96-well plates coated with 100 μg/ml poly-lysine and 20 μg/ml laminin. The plating density was 20,000 cells per well. First, 200 μl Dulbecco's modified Eagle's medium (DMEM) was applied per well (supplemented with 1% penicillin-streptomycin and 1% L-glutamine and 2% B27). After incubation for 2 h at 37° C./5% CO₂, the medium was exchanged with the pre-warmed Neurobasal medium including 1% penicillin-streptomycin, 1% L-glutamine, and 2% B27. The cells—predominantly neurons and astrocytes—were incubated at 37° C. in 5% CO₂ humidified atmosphere for 10 days before administration of reagents.

MTT Assay

The assay was performed as described elsewhere with minor modifications (Khalid et al., 2018). Primary neural cells (see above) grown in a 96-well plate were exposed to drugs/vehicles for 48 h at 37° C./5% CO₂. After aspiration of culture media, a washing was done with 100 μl phosphate buffered saline (PBS, pH=7.2). Then, PBS was completely removed, and 100 μl phenol red-free pre-warmed Neurobasal media and 50 μl MTT solution (3-(4,5-dimethylthiazol-2-yl)-2,5-diphenyltetrazolium bromide, 5 mg/ml; dissolved in PBS) were added to each well and plates were incubated for 4 h in an incubator. After aspiration of solution and a washing with 100 μl PBS, 100 μl dimethyl sulfoxide (DMSO) was added to each well, followed by shaking of the plate in orbital shaker for 10 min. DMSO destroyed the cells and released the formazan, which was detected by optical density value at 560 nm, measured using a plate reader. Optical density values for wells containing cells were corrected by subtracting values measured in cell-free wells prepared and treated in the same way as wells containing control cells.

### Results

### Example 1: Inhibition of GluN2B-EPSCs in Prefrontal Cortex Slices by polySia Fragments

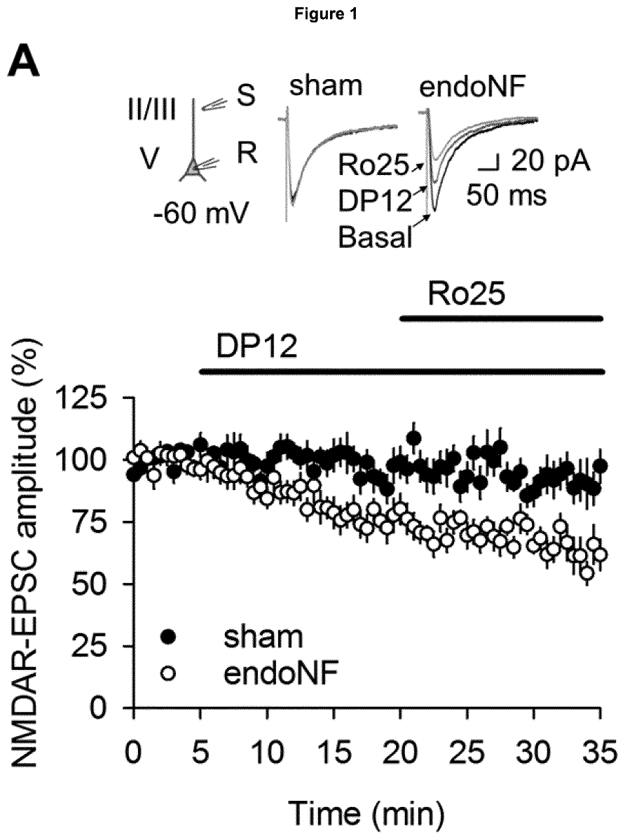

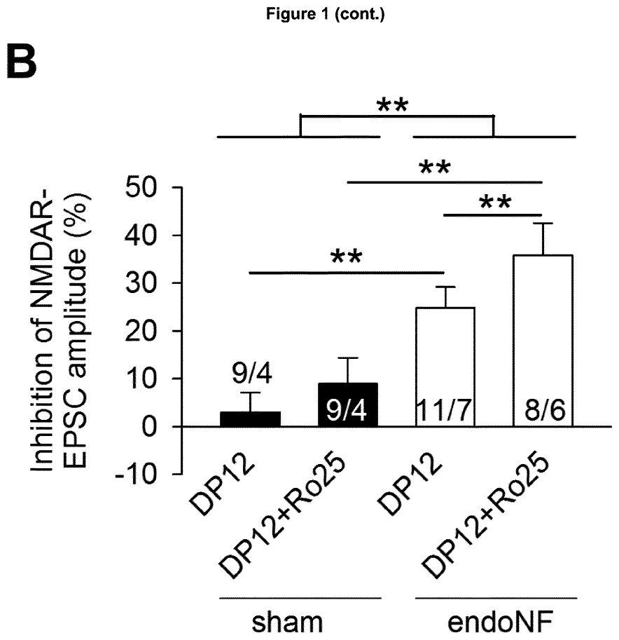

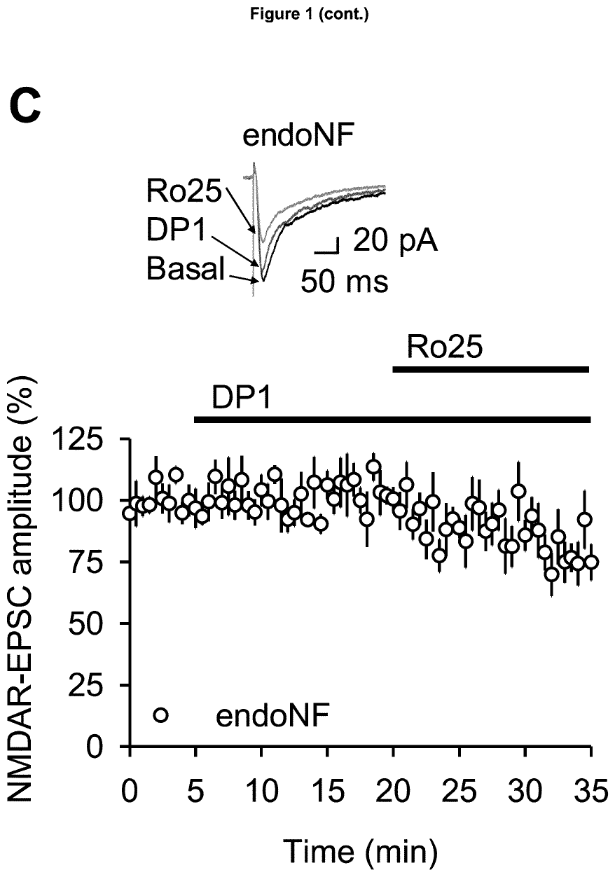

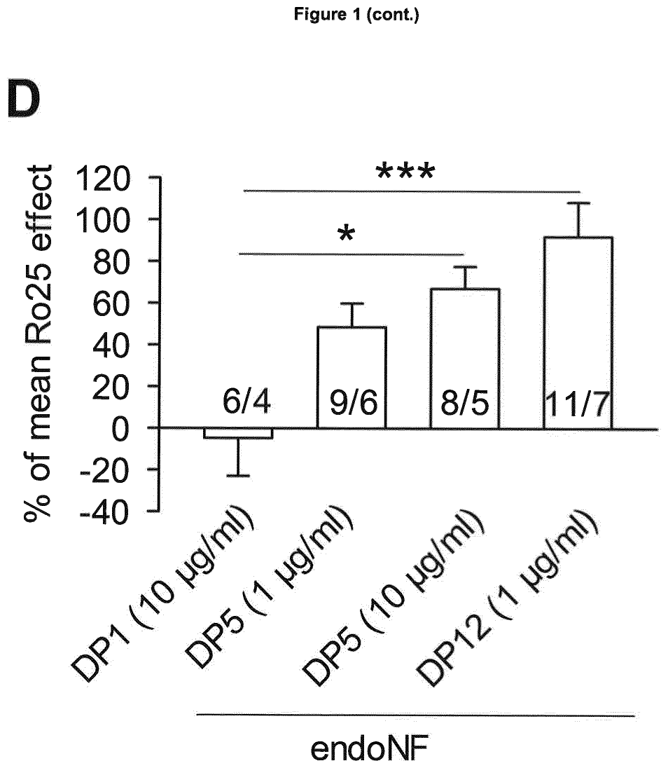

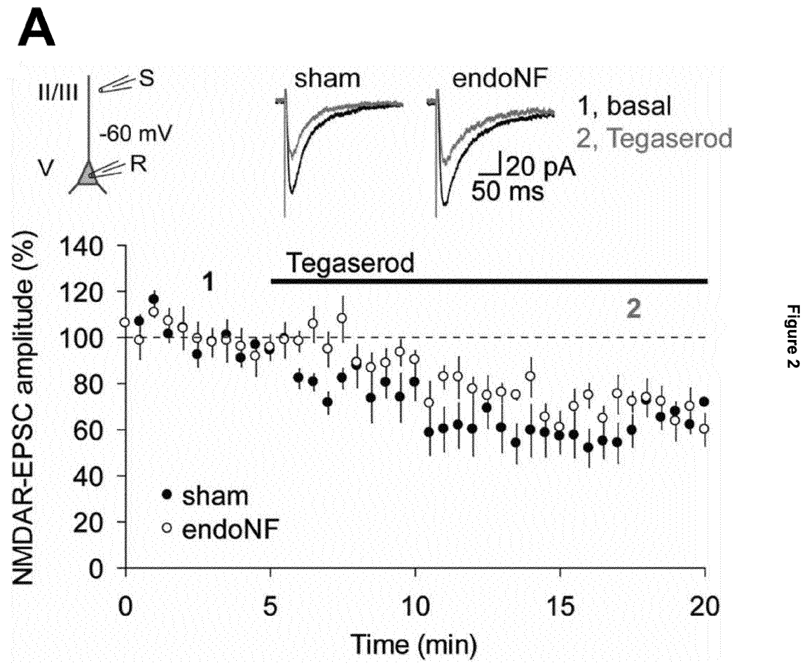

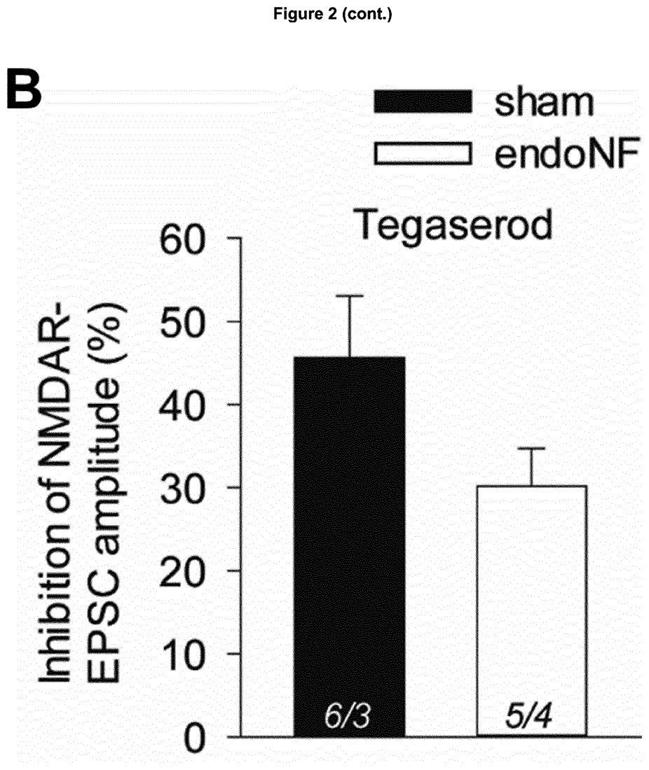

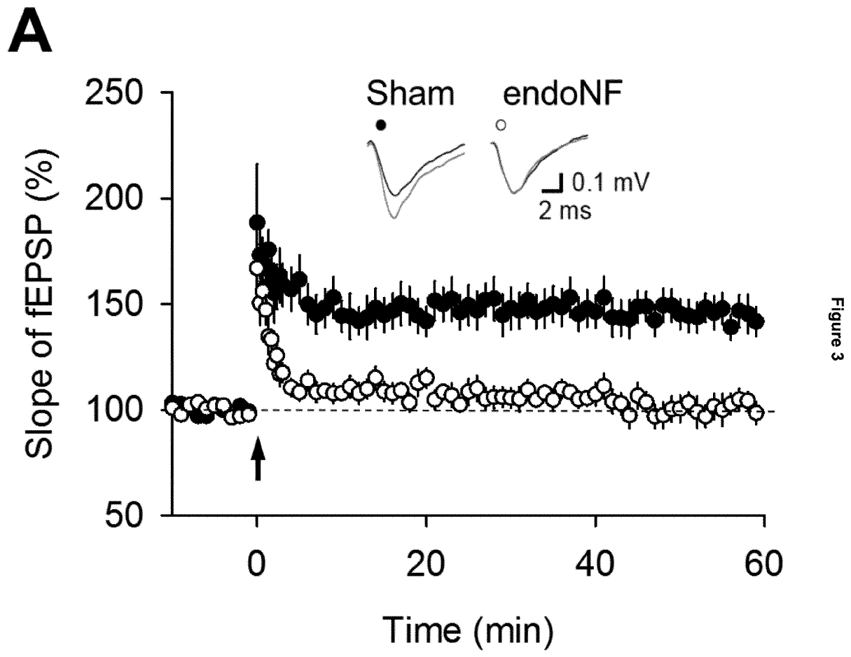

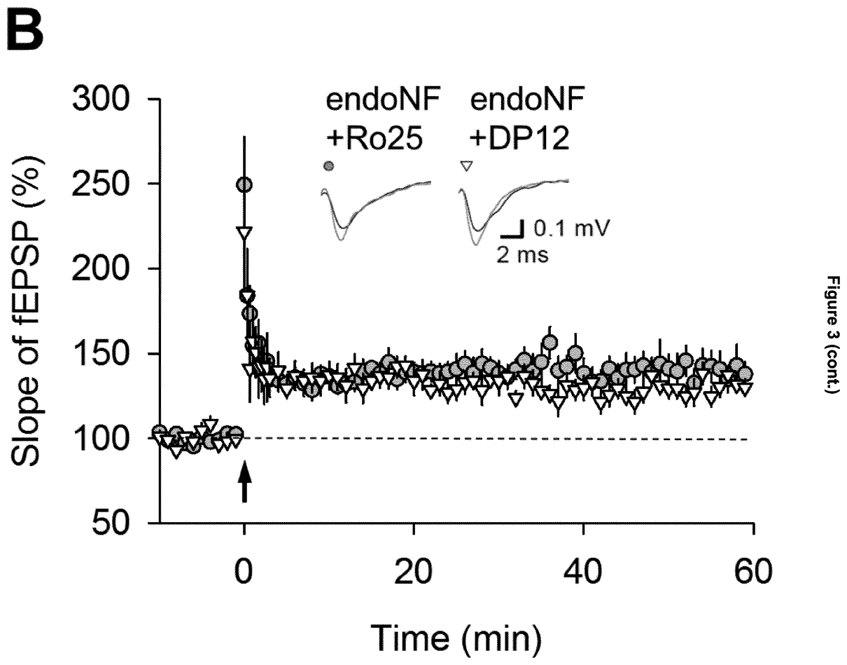

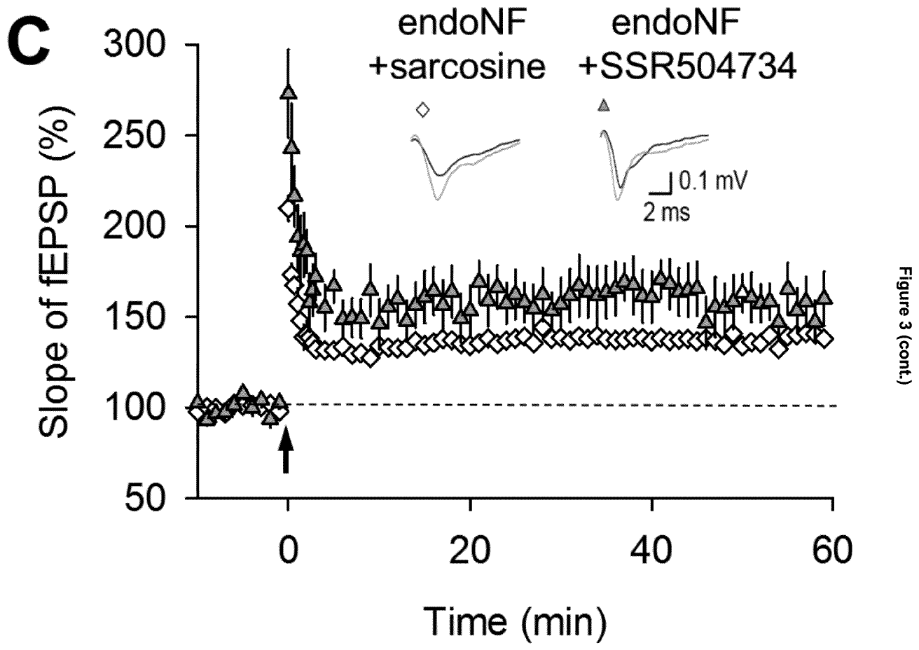

We isolated evoked NMDAR-EPSCs in mPFC slices and tested whether DP12 would occlude their inhibition by Ro 25-6981 (FIG. 1(A, B)). In sham-treated slices, we found no significant inhibition of NMDAR-EPSC amplitude by DP12 or subsequently applied 0.3 μM Ro 25-6981 to inhibit GluN1/GluN2B receptors. In contrast, in endoNF-treated slices, DP12 inhibited significantly NMDAR-EPSC amplitude (˜25% decrease) at concentration of 1 μg/ml. Subsequent application of Ro 25-6981 reduced NMDAR-EPSC amplitude marginally (FIG. 1(A, B)). We estimated that inhibition by DP12 reached˜90% of the mean inhibition produced by Ro 25-6981 applied alone to endoNF-treated slices in the same time window as DP12 (FIGS. 1(D) and 2(D)). Thus, DP12 and 0.3 μM Ro 25-6981 inhibit NMDAR-EPSCs to a similar extent in endoNF-treated slices.

To test whether the effects of the negatively charged DP12 were specific, we repeated these experiments using monomers of sialic acid (DP1), which are also negatively charged but do not inhibit NMDAR-mediated currents in cultured hippocampal neurons (Hammond et al., 2006). DP1 had no effect on the amplitude of NMDAR-EPSCs in endoNF-treated mPFC slices (FIG. 1(C, D)), whereas the subsequent application of 0.3 μM Ro 25-6981 significantly reduced (˜20%) the EPSC amplitude (FIG. 1(C)). This effect was comparable with the level of inhibition by Ro 25-6981 in endoNF-treated slices when applied directly after baseline.

Next, we tested the impact of shorter sialic acid oligomers such as DP5. DP5 (1 μg/ml and 10 μg/ml) reduced the amplitude of NMDAR-EPSCs in endoNF-treated slices, and this effect was significantly different from that of DP1 (FIG. 1(D)). This suggests that five residues of sialic acid are sufficient to inhibit evoked NMDAR-EPSCs, although less efficiently than fragments comprising 12 residues.

Another attractive option to compensate for polySia deficiency would be to use small polySia mimetics. One of these is tegaserod, a serotonin (5-HT₄) receptor agonist (Liu et al., 2005) that is a clinically approved drug for the treatment of irritable bowel syndrome and constipation (Muller-Lissner et al., 2001). Molecular modeling and docking studies have shown that tegaserod might potentially compete with polySia for binding to the polySia binding site of antibody 735 (Bushman et al., 2014). The polySia-mimicking activity of tegaserod to stimulate peripheral nerve regeneration has been shown in vitro and in vivo and found to be independent of its described function as a 5-HT₄ receptor agonist (Bushman et al., 2014). Given the potential of polySia to regulate GluN2B-NMDARs, we examined whether tegaserod may exhibit similar inhibitory effects on NMDAR-EPSCs. Whole-cell recordings in the presence of a 5-HT₄ antagonist revealed strong inhibition of NMDAR-EPSC amplitude by tegaserod in both sham-(˜50%) and endoNF-treated slices (˜30%), which were not significantly different from each other (FIG. 2). These results suggest that tegaserod does not act specifically on GluN2B-mediated synaptic transmission and thus does not mimic the effects of polySia on NMDARs. Hence, it is important to validate the use of other neural polySia mimetics, like DP12, that would more efficiently restore the synaptic function.

### Example 2: Impaired LTP after endoNF Treatment is Restored by DP12

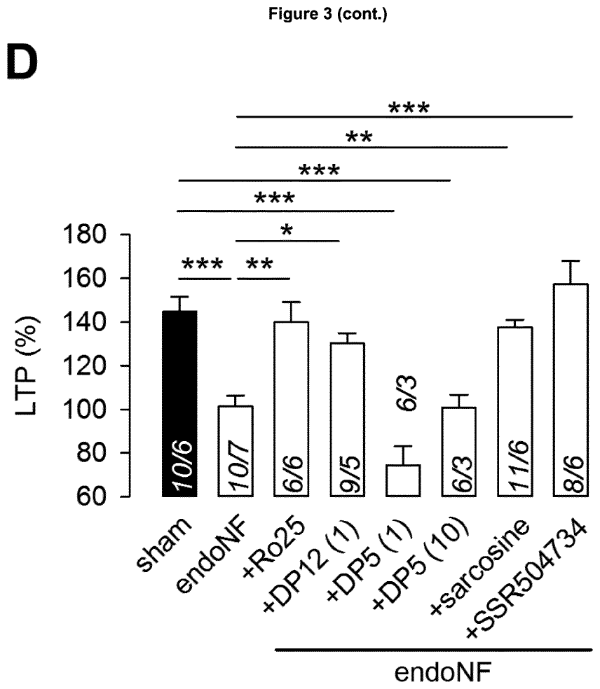

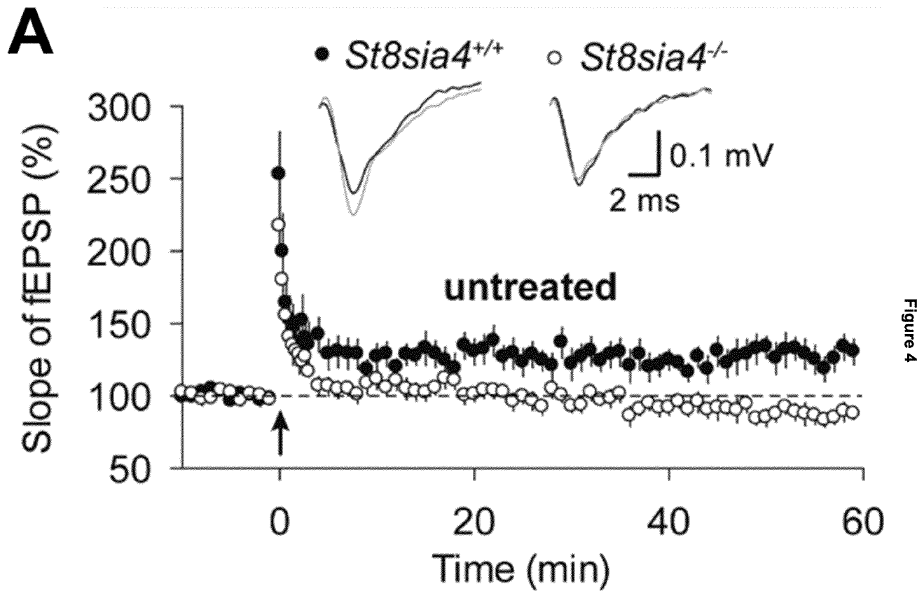

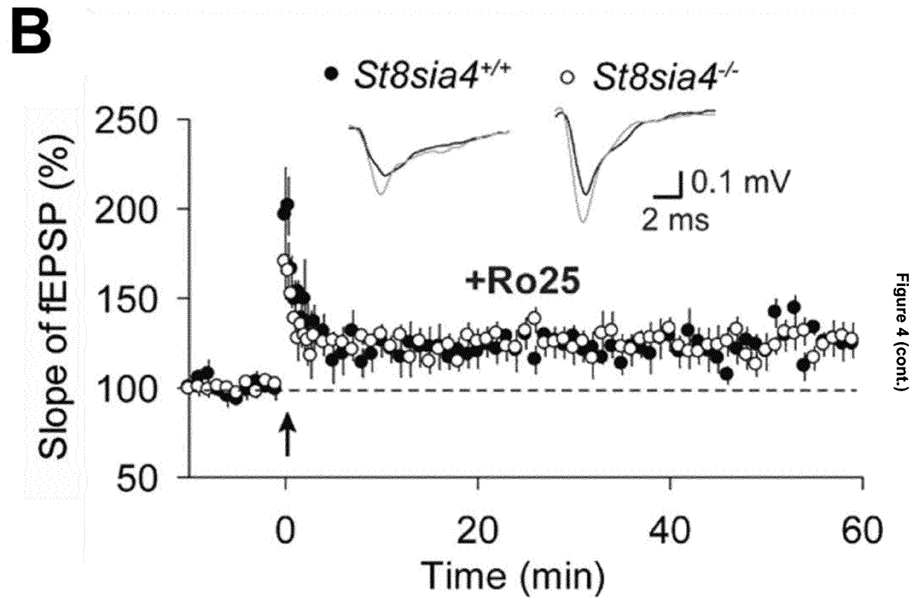

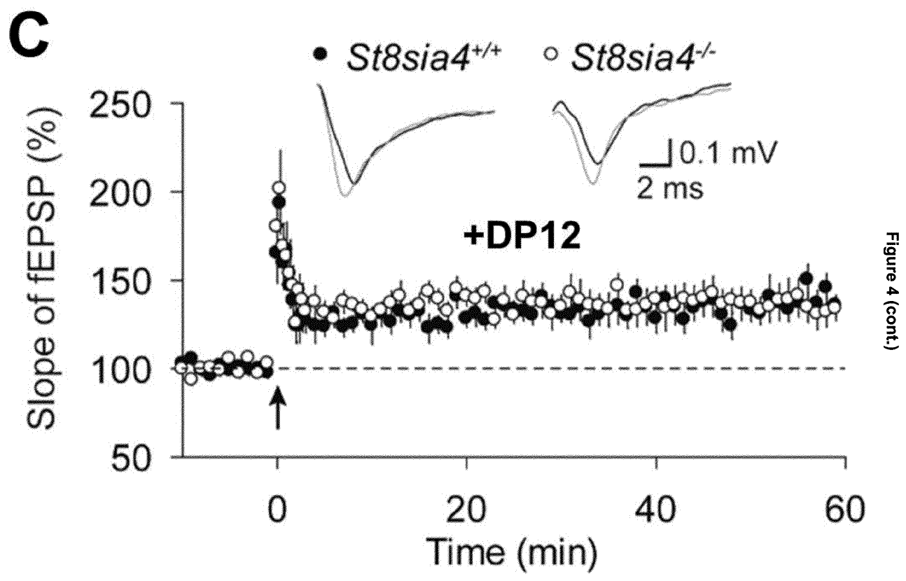

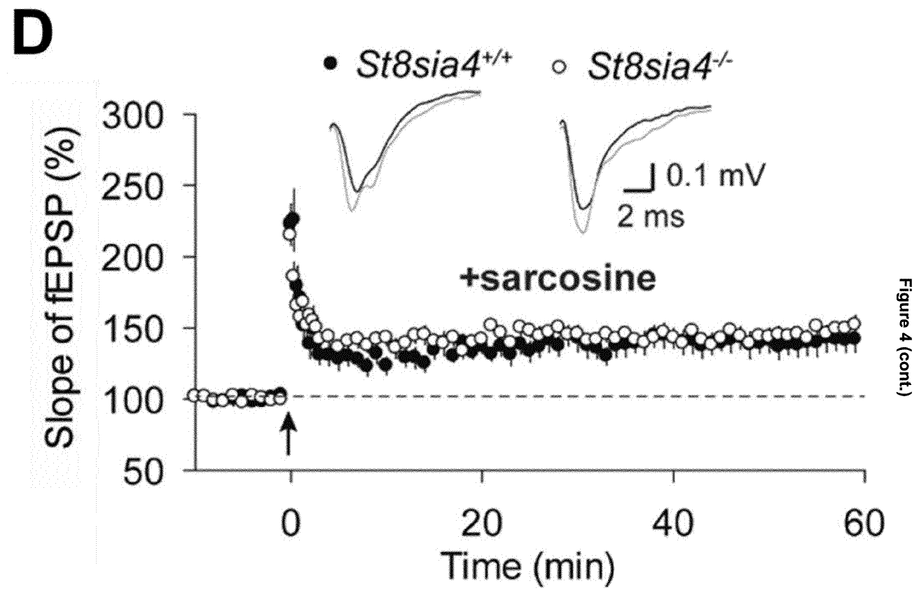

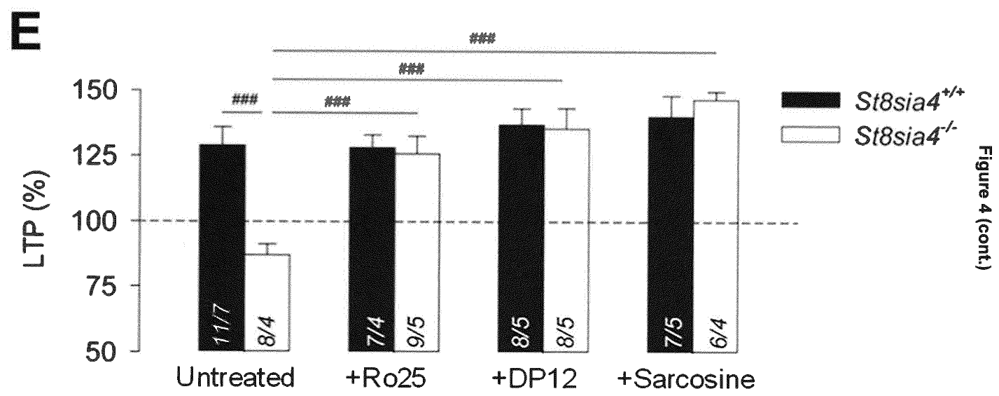

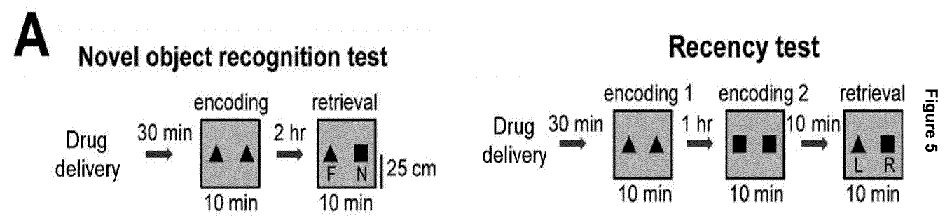

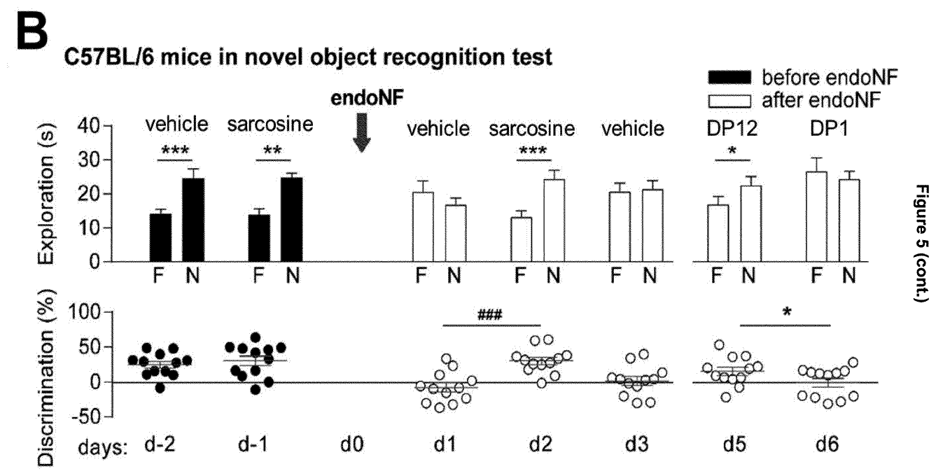

To investigate whether a loss of polySia might affect synaptic plasticity in the prefrontal cortex, we recorded theta-burst stimulation (TBS)-induced LTP from layer II/III—layer V synapses in sham- and endoNF-treated mPFC slices from C57BL/6 mice. TBS induced stable LTP in mPFC of sham-treated slices; however, LTP was impaired in slices treated with endoNF (FIG. 3(A, D)). EndoNF- and sham-incubated slices exhibited similar stimulus-response curves, indicating that the effects of polySia removal on LTP are not due to altered basal synaptic transmission.

Because our patch-clamp recordings revealed increased GluN2B-mediated transmission in endoNF slices and similar inhibition of NMDAR-EPSCs by Ro 25-6981, DP12, and DP5 (FIG. 1(D)), we investigated whether these pharmacological treatments may rescue abnormal LTP by suppressing GluN2B-NMDARs. Field EPSP recordings revealed a full restoration of LTP levels in endoNF-treated slices perfused with Ro 25-6981 or DP12 (FIG. 3(B, D)), indicating that DP12 was as effective as Ro 25-6981 in restoring LTP. In sham-treated slices, we observed no effect of Ro 25-6981 on LTP magnitude, suggesting that GluN1/GluN2B-NMDARs do not contribute to LTP induction in the prefrontal cortex in our recording conditions. In contrast to DP12, DP5 (at 1 or 10 μg/ml) did not rescue LTP in endoNF-incubated slices (FIG. 3(D)). We thus conclude that DP5 is not sufficient to mimic functions of neural polySia, while DP12 is.

Next, we explored the possibility that impaired LTP may be rescued by improving the ratio between synaptic and extra synaptic NMDAR-mediated transmission by increasing synaptic NMDAR transmission with the clinically used glycine transporter type 1 (GlyT1) inhibitor sarcosine (Javitt, 2012). Strikingly, in the presence of sarcosine, the LTP magnitude in endoNF-treated slices was fully restored to sham levels (FIG. 3(C, D)). In agreement with these data, another GlyT1 inhibitor SSR 504734 (Depoortere et al., 2005) (3 μM), which is more potent and specific than sarcosine, also rescued LTP in endoNF-treated slices (FIG. 3(C, D)).

### Example 3: Restoration of LTP in ST8SIA4-Deficient Mice by DP12

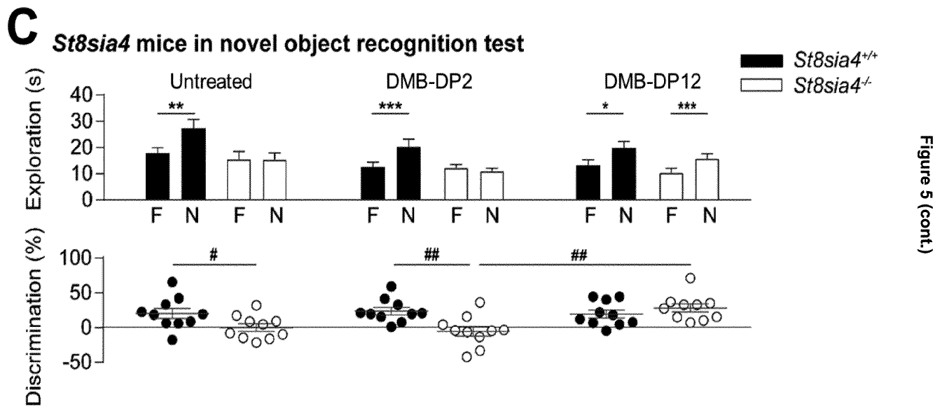

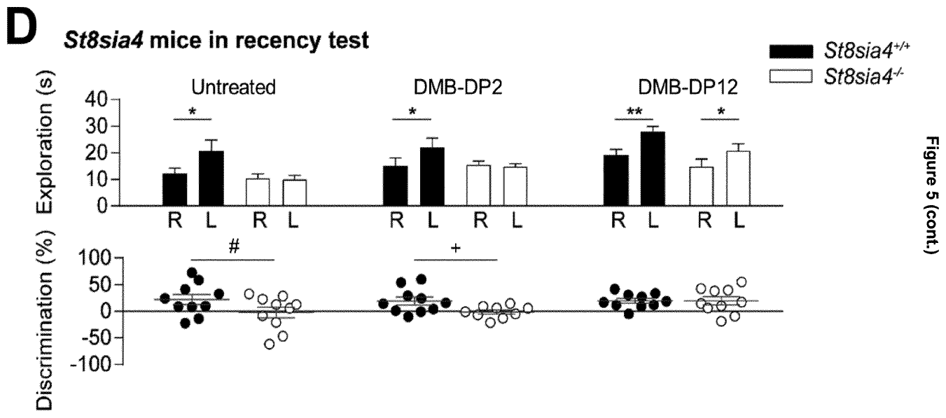

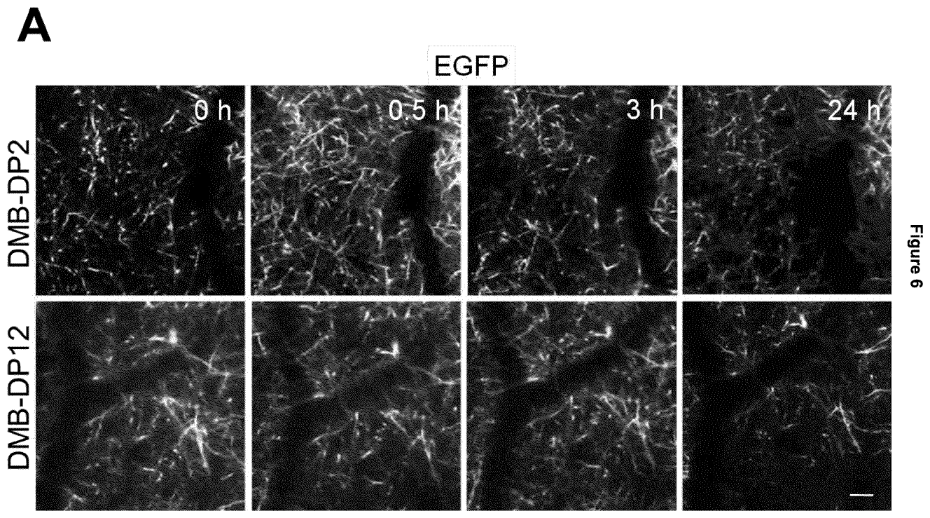

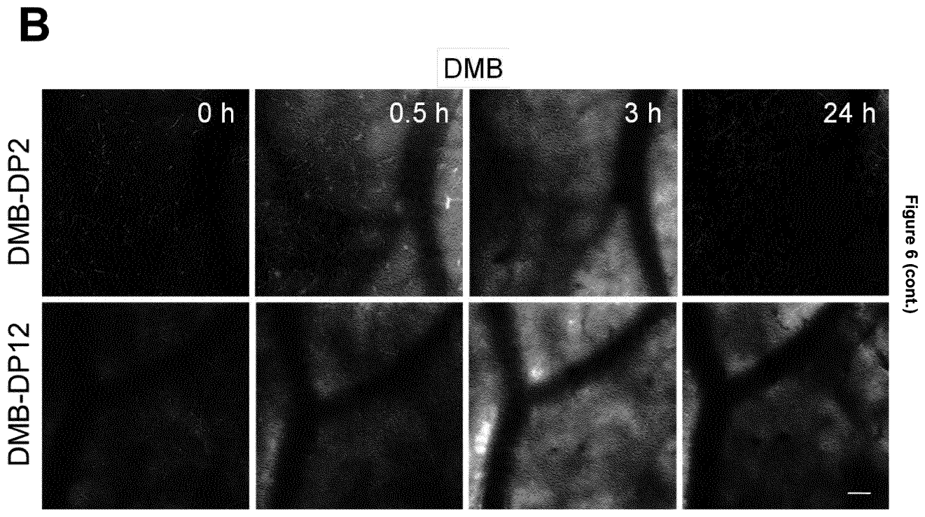

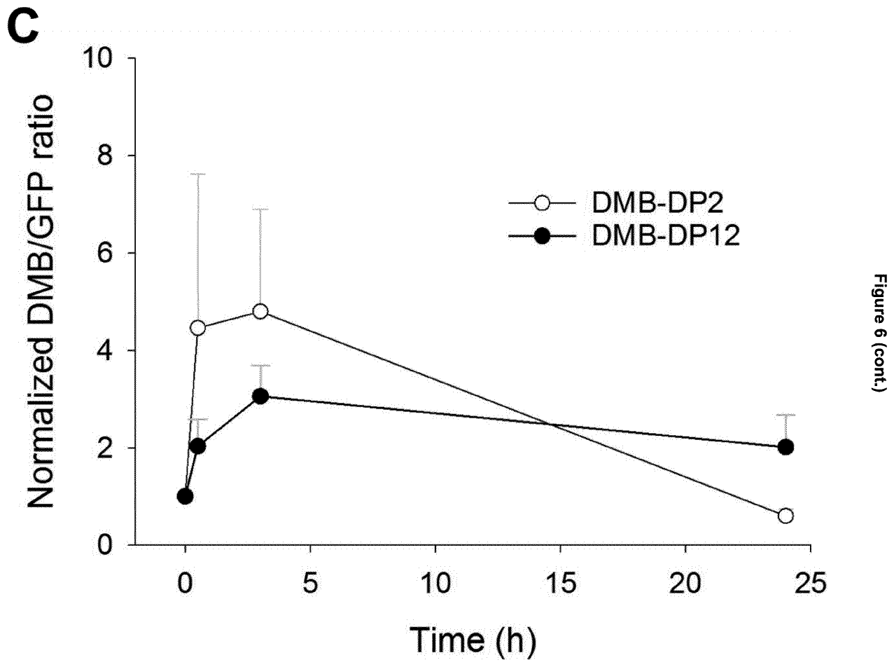

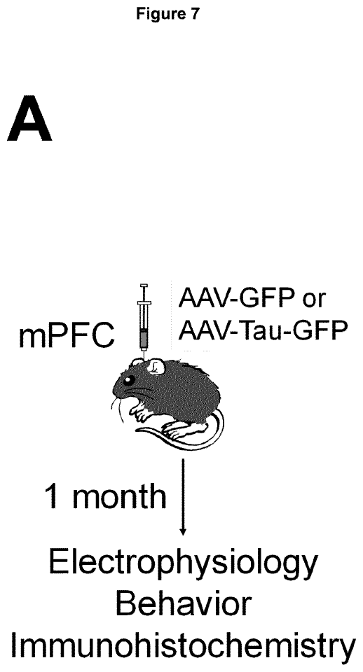

Because acute removal of polySia led to elevated synaptic transmission through GluN2B-NMDARs, we wondered whether this might also be the case in mice that are constitutively deficient in the polysialyltransferase ST8SIA4 (St8sia4^{-/-}) and that show no detectable polySia in the mPFC during adulthood (Eckhardt et al., 2000; Nacher et al., 2010). Extracellular fEPSP recordings demonstrated a pronounced decrease in LTP magnitude (by ˜40%) in St8sia4^{-/-} mice compared with St8sia4^{+/+} mice (FIG. 4(A, E)), which resembled the LTP deficits found in endoNF-treated slices (FIG. 3(A, D)). This result could be reproduced in an independent set of recordings on another setup and by another set of LTP experiments performed at 35° C. (data not shown).

In agreement with our endoNF data, we observed full restoration of LTP levels in mPFC slices from St8sia4^{-/-} mice in the presence of either Ro 25-6981 (FIG. 4(B, E)) or DP12 (FIG. 4(C, E)), or sarcosine (FIG. 4(D, E)). Notably, Ro 25-6981, DP12, and sarcosine had no effect on the LTP magnitude in St8sia4^{+/+} mice (FIG. 4(B-D)). These results indicate that resetting the balance between synaptic and extra synaptic NMDAR activation may normalize synaptic plasticity in St8sia4^{-/-} mice.

### Example 4: Impaired Object Recognition Memory in polySia-Depleted Mice is Normalized by DP12

The prefrontal cortex is known to be involved in object recognition memory in rodents (Barbosa et al., 2013). Because LTP was impaired in mPFC slices treated with endoNF, we asked whether acute removal of polySia in the prefrontal cortex would affect this form of learning and memory. Thus, we performed a longitudinal experiment comparing performance of C57BL/6J mice before and after endoNF injection into the mPFC in vivo, which efficiently digested polySia (FIG. 5(A)). Two days before administration of endoNF, mice showed normal object recognition being i.p. injected with either vehicle (d-2) or sarcosine (d-1) 30 min before the encoding phase (FIG. 5(B)). However, one day after injection of endoNF into mPFC (d1), the same mice were impaired in discriminating novel versus familiar objects (FIG. 5(B)). This deficit was fully alleviated by i.p. injection of sarcosine on day 2 (d2). On the day after sarcosine injection (d3), impairment in novel object recognition was reinstated in endoNF-treated mice, which is consistent with the short lifetime of sarcosine (Rehberg and Gerritsen, 1968) and the fact that the recovery of polySia expression lasts more than 1 week after a single endoNF injection (Seki and Rutishauser, 1998). Because DP12 rescued LTP in vitro, we also tested its effects in vivo. To improve the delivery of DP12 to the brain, we applied it intranasally 30 min before the encoding phase. DP12 significantly improved novel object recognition (d5), while administration of DP1 on day 6 (d6) had no effect (FIG. 5(B)).

### Example 5: Delivery of DP12 to mPFC by Intranasal Administration

To ensure that DP12 indeed penetrates to the brain after intranasal delivery, we conjugated DP12 with a fluorescent dye, 1,2-diamino-4,5-methylenedioxybenzene.2HCl (DMB), and performed live two-photon imaging of DMB-DP12 conjugates in the mPFC of Thy1-EGFP mice. The EGFP signal (FIG. 6(A)) was used for finding the same position during repetitive imaging and for normalization of DMB signal (FIG. 6(B)). We observed a rapid penetration of both DMB-DP12 and the control reagent DMB-DP2 already at 30 min after intranasal delivery, which was followed by a further increase in the DMB/EGFP signal at 3 h after delivery (FIG. 6(C)). We detected a decline of the signals at 24 h after intranasal application.

### Example 6: Impaired Object Recognition Memory in polySia-Deficient Mice is Normalized by DP12

Here, we attempted to rescue cognitive functions in St8sia4^{-/-} mice by DMB-DP12 delivery. Since we observed a positive effect of DP12 in endoNF-treated mice with 30 min interval between intranasal delivery and encoding phase (FIG. 5(B)), we continued to use this time interval. St8sia4^{-/-} mice showed impaired novel object recognition, which was fully rescued by intranasal delivery of DMB-DP12 but not by control DMB-DP2 (FIG. 5(C)). To provide further support to the idea that DP12 can rescue mPFC function, we examined performance of St8sia4^{-/-} mice using the mPFC-dependent recency task (FIG. 5(A), right). St8sia4^{+/+} mice showed a clear preference for the least as compared to the most recently explored object in the retrieval phase. In contrast, St8sia4^{-/-} mice explored the two objects equally (FIG. 5(D)). Their performance in this task could be restored by DMB-DP12, but not by DMB-DP2.

### Example 7: Impaired mPFC LTP and Recent Object Recognition Memory in Mice Overexpressing GFP-Tau[R406W] are Normalized by DP12

As overexpression of human Tau mutants was reported to lead to upregulation of extra synaptic GluN2B-mediated signaling, we attempted to rescue synaptic and cognitive functions in the mouse model of tauopathy. Immunohistochemical analysis showed that mice infected with AAV-GFP-Tau[R406W] in the mPFC showed a strong upregulation of Tau phosphorylation one month after injection (FIG. 7(A)) as compared to GFP-expressing mice (FIG. 7(B)). AAV-GFP-Tau[R406W] infected mice had impaired mPFC LTP, but bath application of DP12 could fully rescue LTP (FIG. 7(C)): The levels of LTP after this treatment were not different from those in control AAV-GFP expressing mice. Consistent with data shown in FIG. 5, control AAV-GFP-injected mice showed discrimination between recent (R) and less recent (L) objects. Untreated AAV-GFP-Tau[R406W] injected mice showed impaired recent object recognition, which was fully rescued by intranasal delivery of DP12, but not by control DP1 (FIG. 7(D)).

### Example 8: Increase of LTP in CA3-CA1 Synapses in Slices from 5×FAD Mice after Treatment with DP12

Finally, we tested if DP12 would have an effect in the 5×FAD mouse model of AD associated with amyloidosis and upregulation of extracellular glutamate and signaling through extra synaptic GluN2B receptors. Using a special slice incubation chamber with improved oxygen supply, we were able to induce LTP in CA3-CA1 synapses in slices from 5×FAD mice (FIG. 8). Still, the levels of LTP could be strongly increased by bath application of DP12, confirming that DP12 is efficient treatment to improve synaptic functions in the hippocampus.

### Example 9: Impaired Recent Object Recognition Memory in 5×FAD Mice is Normalized by DP12

As the recency test was consistently used for analysis of DP12 effects in other mouse models, it was further investigated whether 13- to 14-month-old 5×FAD mice are impaired in this form of memory. In this series of experiments, the intervals between each session during recency test were increased to 90 min to increase the difficulty to recall the temporal order of the two objects shown in the arena (FIG. 12(A)). It was found that 5×FAD mice spent equal exploration time near the most recently and less recently explored objects and showed no discrimination between objects in contrast to age-matched wild-type mice from the same breeding (FIG. 12(B)). To verify whether DP12 or avDP10-9OAc may improve cognitive function in the 5×FAD model of AD, these compounds or a vehicle (H₂O) were intranasally delivered in the same cohort of 5×FAD mice before multiple recency test sessions (separated by 4 day-interval). The numbers of mice in each treatment group during one session were counterbalanced and each mouse received in a random order both drugs and the vehicle. In this series of experiments, it was relied on the time-course of DP12 delivery to the brain as shown in FIG. 6, and hence the interval between drug delivery and the beginning of the recency test was increased to 2 hours (i.e. at the estimated peak of drug concentration), while a lower concentration of drug was used (0.5 mg/kg for DP12 and equimolar concentration for avDP10-9OAc). Excitingly, 5×FAD mice treated with DP12 showed a clear preference for the least recently explored object as compared to the most recently explored object in the retrieval phase (FIG. 12(C)). In contrast, vehicle-treated 5×FAD mice explored the two objects equally (FIG. 12(C)). There was a significant difference in object discrimination after treatment with DP12 as compared to vehicle. avDP10-9OAc possibly improved discrimination between objects in a fraction of mice. Noteworthy, four avDP10-9OAc-treated mice showed discrimination more than 20%, while only two of vehicle-treated mice showed comparable levels of discrimination. DP12 treatment affected most mice (10 of 11 mice showed discrimination more than 20%).

### Example 10: DP12 and avDP10-9OAc do not Affect the Cell Viability, while DP10 Improves it

To test if DP12, avDP10-9OAc or DP10 affect neural cell viability in the range of physiologically relevant concentrations from 30 to 1000 nM, the compounds were applied to dissociated hippocampal cultures on 10th day in vitro for 48 hours after that the cell viability was measured using the MTT assay. No effects of DP12 and DP10-9OAc were detected, thus confirming that these compounds are not toxic. Surprisingly, DP10 had a strong potentiating effect on cell viability at concentration of 30, 100 and 300 nM (FIG. 13). Because application of Ro25 had no effects on cell viability, it was concluded that the DP10 effects on cell viability are not mediated by inhibition of GluN2B-containing NMDA receptors, but there are additional mechanisms that may potentiate therapeutic effects of some of the claimed compounds, including DP10.

### Example 11: Impaired Recent Object Recognition Memory in 5×FAD Mice is Normalized by DP10 but not DP20

The positive effect of DP10 on cell viability in vitro stimulated an interest to test its effects in vivo. For comparison, DP20 was included, as one of previously patented polySia variants with immunological activities. The design of experiment was as shown in FIG. 12(A). To verify whether DP10 or DP20 (taken in equimolar concentration corresponding to 0.5 mg/kg of DP12) may improve cognitive function in the 5×FAD model of AD, these compounds or a vehicle (H₂O) were intranasally delivered in the same cohort of 5×FAD mice before multiple recency test sessions (separated by a 4 day-interval). 5×FAD mice treated with DP10 showed a preference for the least recently explored object as compared to the most recently explored object in the retrieval phase (FIG. 14, upper panel). In contrast, vehicle-treated 5×FAD mice explored the two objects equally (FIG. 14, upper panel). DP20 had a tendency to improve discrimination as compared to vehicle, but mice treated with DP10 were significantly better in object discrimination as compared to both vehicle and DP20-treated groups (P<0.05; FIG. 14, lower panel). It is plausible to assume that the lesser potency of DP20 as compared to DP10 to improve cognitive function is due to a large size of DP20, which may reduce its efficacy for intranasal delivery to the brain.

The Invention is Further Characterized by the Following Items:

1. A polysialic acid according to the general formula (1) as given as follows and derivatives thereof:

(α(2→8)Neu5Ac)ₙ (1)

wherein Neu5Ac is N-acetylneuraminic acid, and
n is an integer in the range from 6 to 13,
for use in the prevention or treatment of a neurological and neuropsychiatric disorder,
wherein said derivatives are substituted with at least one sugar, acetyl group, or acyl group at at least one monomer of the polysialic acid,
optionally linked to at least one nano-carrier,
and pharmaceutically acceptable salts thereof.
2. The polysialic acid of item 1, wherein n is an integer in the range from 10 to 13.
3. The polysialic acid of item 1 or 2, wherein the polysialic acid inhibits the activation of heterodimeric GluN1/GluN2B or heterotrimeric GluN1/GluN2A/GluN2B-containing NMDA receptors.
4. The polysialic acid of any one of the preceding items, wherein said derivatives are substituted with at least one sugar, acetyl group, or acyl group at one monomer of the polysialic acid.
5. The polysialic acid of any one of the preceding items, wherein said derivatives are substituted with at least one sugar, acetyl group or acyl group at the one monomer at the non-reducing end of the polysialic acid.
6. The polysialic acid of any one of the preceding items, wherein the at least one sugar is glucose, N-acetylglucosamine, N-acetylgalactosamine, galactose, fucose, mannose or xylose.
7. The polysialic acid of any one of the preceding items, wherein said derivatives thereof have the formula (2) as given as follows:

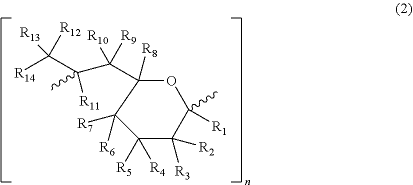

wherein at the one monomer at the non-reducing end of the polysialic acid:
i) R₁ is a carboxyl group;
ii) each of R₂, R₃, R₅, R₇, R₈, R₁₀, R₁₂, R₁₃ is independently from each other a hydrogen;
iii) R₄ is a hydroxyl group or an O-acetyl group;
iv) R₆ is NHCOCH₃;
v) R₉ is a hydroxyl group or an O-acetyl group;
vi) R₁₁ is a hydroxyl group or hydrogen; and
vii) R₁₄ is a hydroxyl group or an O-acetyl group.
8. The polysialic acid of item 7, wherein at the one monomer at the non-reducing end of the polysialic acid:
i) R₄ is a hydroxyl group;
ii) R₆ is NHCOCH₃;
iii) R₉ is a hydroxyl group or an O-acetyl group; and
iv) R₁₄ is an O-acetyl group.
9. The polysialic acid of item 7 or 8, wherein n is an integer in the range from 10 to 13.
10. The polysialic acid of any one of the preceding items, wherein the polysialic acid is chemically linked via its reducing end to a nano-carrier.
11. The polysialic acid of any one of the preceding items, wherein the neurological and neuropsychiatric disorder is schizophrenia, tauopathy, bipolar disorder, depression, epilepsy, Huntington's disease, multiple sclerosis, amyotrophic lateral sclerosis or stroke.
12. The polysialic acid of item 11, wherein tauopathy comprises Alzheimer's disease, frontotemporal dementia (FTD), primary age-related tauopathy (PART), chronic traumatic encephalopathy, progressive supranuclear palsy (PSP), corticobasal degeneration, dementia with Lewy Bodies, frontotemporal dementia and parkinsonism linked to chromosome 17 (FTDP-17), argyrophilic grain disease (AGD), glial globular tauopathy, Pick's diseases, Parkinson's disease.
13. A pharmaceutical composition comprising as an active ingredient the polysialic acid and/or derivatives and/or pharmaceutically acceptable salts thereof according to any one of the preceding items, optionally comprising a pharmaceutical acceptable carrier.
14. The pharmaceutically composition of item 13, wherein the pharmaceutically composition comprises a pharmaceutically acceptable carrier, wherein the pharmaceutical acceptable carrier is a solid pharmaceutical acceptable carrier, preferably lactose, terra alba, sucrose, talc, gelatin, agar, pectin, acacia, magnesium stearate or stearic acid, a liquid pharmaceutical acceptable carrier, preferably sugar syrup, peanut oil, olive oil or water, or a gaseous pharmaceutical acceptable carrier, preferably carbon dioxide or nitrogen.
15. The pharmaceutical composition of item 13 or 14, wherein the pharmaceutical composition is administered intranasal, oral, dermal, rectal, parenteral, preferably intranasal.
16. The pharmaceutical composition of item 15, wherein parenteral administration comprises intravitreal injection, subcutaneous injection, intravenous injection, intraperitoneal injection, intramuscular injection, perfusion, infusion or topical administration.
17. The pharmaceutical composition of any one of items 13 to 16, for use in the prevention or treatment of a neurological and neuropsychiatric disorder.
18. The pharmaceutical composition of item 17, wherein the neurological and neuropsychiatric disorder is schizophrenia, tauopathy, bipolar disorder, depression, epilepsy, Huntington's disease, multiple sclerosis, amyotrophic lateral sclerosis, or stroke.
19. The pharmaceutical composition of item 18, wherein tauopathy comprises Alzheimer's disease, frontotemporal dementia (FTD), primary age-related tauopathy (PART), chronic traumatic encephalopathy, progressive supranuclear palsy (PSP), corticobasal degeneration, frontotemporal dementia, dementia with Lewy Bodies, frontotemporal dementia and parkinsonism linked to chromosome 17 (FTDP-17), argyrophilic grain disease (AGD), glial globular tauopathy, Pick's diseases, and Parkinson's disease.
20. A method of producing the polysialic acid and/or derivatives and/or pharmaceutically acceptable salts thereof according to any one of items 1-12, comprising:
a) dissolving colominic acid in acetic acid;
b) stopping the reaction of step a) with NaOH;
c) storing the mixture of step b);
d) separating oligo- and polymers by chromatography with a NaCl gradient;
e) obtaining the polysialic acid and/or derivatives and/or pharmaceutically acceptable salts thereof according to any one of items 1-12.

### REFERENCES

Ansel H. C. and Loyd A. (2013), Pharmaceutical Dosage Forms and Drug Delivery Systems, 7^{th} edition, Lippincott Williams & Wilkins Publishers, 1999.
Barbosa, F. F., Santos, J. R., Meurer, Y. S., Macedo, P. T., Ferreira, L. M., Pontes, I. M., Ribeiro, A. M., and Silva, R. H. (2013). Differential Cortical c-Fos and Zif-268 Expression after Object and Spatial Memory Processing in a Standard or Episodic-Like Object Recognition Task. Frontiers in behavioral neuroscience 7, 112.
Berge, S. M., Bighley, L. D. and Monkhouse D. C. (1977). Pharmaceutical salts. Journal Pharm. Sci., 66(1), 1-19.
Brennaman, L. H., Kochlamazashvili, G., Stoenica, L., Nonneman, R. J., Moy, S. S., Schachner, M., Dityatev, A., and Maness, P. F. (2011). Transgenic mice overexpressing the extracellular domain of NCAM are impaired in working memory and cortical plasticity. Neurobiology of disease 43, 372-378.
Bushman, J., Mishra, B., Ezra, M., Gul, S., Schulze, C., Chaudhury, S., Ripoll, D., Wallqvist, A., Kohn, J., Schachner, M., et al. (2014). Tegaserod mimics the neurostimulatory glycan polysialic acid and promotes nervous system repair. Neuropharmacology 79, 456-466.
Chard T. (1980). Ammonium sulphate and polyethylene glycol as reagents to separate antigen from antigen-antibody complexes. Methods Enzymol., 70, 280-91.
Chen, L., Muhlhauser, M., and Yang, C. R. (2003). Glycine tranporter-1 blockade potentiates NMDA-mediated responses in rat prefrontal cortical neurons in vitro and in vivo. Journal of neurophysiology 89, 691-703.
Depoortere, R., Dargazanli, G., Estenne-Bouhtou, G., Coste, A., Lanneau, C., Desvignes, C., Poncelet, M., Heaulme, M., Santucci, V., Decobert, M., et al. (2005). Neurochemical, electrophysiological and pharmacological profiles of the selective inhibitor of the glycine transporter-1 SSR504734, a potential new type of antipsychotic. Neuropsychopharmacology: official publication of the American College of Neuropsychopharmacology 30, 1963-1985.
Eckhardt, M., Bukalo, O., Chazal, G., Wang, L., Goridis, C., Schachner, M., Gerardy-Schahn, R., Cremer, H., and Dityatev, A. (2000). Mice deficient in the polysialyltransferase ST8SialV/PST-1 allow discrimination of the roles of neural cell adhesion molecule protein and polysialic acid in neural development and synaptic plasticity. J Neurosci 20, 5234-5244.
Hildebrandt, H., Muhlenhoff, M., and Gerardy-Schahn, R. (2010). Polysialylation of NCAM. Advances in experimental medicine and biology 663, 95-109.
Finne, J., Leinonen, M., Makela, P. H. (1983). Antigenic similarities between brain components causing meningitis. Implications for vaccine development and pathogenesis. Lancet, 2, 355-7.
Frosch, M., Görgen, I., Boulnois, G. J., Timmis, K. N., Bitter-Suermann, D. (1985). Mutant bacteriophage with non-catalytic endosialidase binds to both bacterial and eukaryotic polysialic acid and can be used as probe for its detection, Proc. Natl. Acad. Sci. USA, 82(4):1194-8, PMID: 3919387.
Hammond, M. S., Sims, C., Parameshwaran, K., Suppiramaniam, V., Schachner, M., and Dityatev, A. (2006). Neural cell adhesion molecule-associated polysialic acid inhibits NR2B-containing N-methyl-D-aspartate receptors and prevents glutamate-induced cell death. J Biol Chem 281, 34859-34869.
Häyrinen, J., Jennings, H., Raff, H. V., Rougon, G., Hanai, N., Gerardy-Schahn, R., Finne, J (1995). Antibodies to polysialic acid and its N-propyl derivative: binding properties and interaction with human embryonal brain glycopeptides. Journal Infect. Dis.; 171(6), 1481-90.
Huang, Y. Y., Simpson, E., Kellendonk, C., and Kandel, E. R. (2004). Genetic evidence for the bidirectional modulation of synaptic plasticity in the prefrontal cortex by D1 receptors. Proceedings of the National Academy of Sciences of the United States of America 101, 3236-3241.
Javitt, D. C. (2012). Glycine transport inhibitors in the treatment of schizophrenia. Handb Exp Pharmacol, 367-399.
Keys, T. G., Fuchs, H. L., Ehrit, J., Alves, J., Freiberger, F., and Gerardy-Schahn, R. (2014). Engineering the product profile of a polysialyltransferase. Nature chemical biology 10, 437-442.
Keys, T. G., Freiberger, F., Ehrit, J., Krueger, J., Eggers, K., Buettner, F. F., and Gerardy-Schahn, R. (2012). A universal fluorescent acceptor for high-performance liquid chromatography analysis of pro- and eukaryotic polysialyltransferases. Analytical biochemistry 427, 107-115.
Khalid, M. K., Asad, M., Henrich-Noack, P., Sokolov, M., Hintz, W., Grigartzik, L., Zhang, E., Dityatev, A., van Wachem, B., Sabel, B. A. (2018). Evaluation of Toxicity and Neural Uptake In Vitro and In Vivo of Superparamagnetic Iron Oxide Nanoparticles. Int J Mol Sci, 19(9).
Kochlamazashvili, G., Senkov, O., Grebenyuk, S., Robinson, C., Xiao, M. F., Stummeyer, K., Gerardy-Schahn, R., Engel, A. K., Feig, L., Semyanov, A., et al. (2010). Neural cell adhesion molecule-associated polysialic acid regulates synaptic plasticity and learning by restraining the signaling through GluN2B-containing NMDA receptors. J Neurosci 30, 4171-4183.
Liu, M., Geddis, M. S., Wen, Y., Setlik, W., and Gershon, M. D. (2005). Expression and function of 5-HT4 receptors in the mouse enteric nervous system. American journal of physiology Gastrointestinal and liver physiology 289, G1148-1163.
Leger, M., Quiedeville, A., Bouet, V., Haelewyn, B., Boulouard, M., Schumann-Bard, P., and Freret, T. (2013). Object recognition test in mice. Nature protocols 8, 2531-2537.
Muller-Lissner, S. A., Fumagalli, I., Bardhan, K. D., Pace, F., Pecher, E., Nault, B., and Ruegg, P. (2001). Tegaserod, a 5-HT(4) receptor partial agonist, relieves symptoms in irritable bowel syndrome patients with abdominal pain, bloating and constipation. Alimentary pharmacology & therapeutics 15, 1655-1666.
Nacher, J., Guirado, R., Varea, E., Alonso-Llosa, G., Rockle, I., and Hildebrandt, H. (2010). Divergent impact of the polysialyltransferases ST8SiaII and ST8SiaIV on polysialic acid expression in immature neurons and interneurons of the adult cerebral cortex. Neuroscience 167, 825-837.
Nelson, A. J., Cooper, M. T., Thur, K. E., Marsden, C. A., and Cassaday, H. J. (2011). The effect of catecholaminergic depletion within the prelimbic and infralimbic medial prefrontal cortex on recognition memory for recency, location, and objects. Behavioral neuroscience 125, 396-403.
Oakley, H., Cole, S. L., Logan, S., Maus, E., Shao, P., Craft, J., et al. (2006). Intraneuronal β-amyloid aggregates, neurodegeneration and neuron loss in transgenic mice with five familial Alzheimer's disease mutations: potential factors in amyloid plaque formation. J. Neurosci. 26, 10129-10140.
Remington, (2012). The Science and Practice of Pharmacy, edited by Allen, Loyd V., Jr., 22^{nd} edition, Pharmaceutical Sciences.
Rehberg, M. L., and Gerritsen, T. (1968). Sarcosine metabolism in the rat. Archives of biochemistry and biophysics 127, 661-665.
Robinson J. R. (1978), Sustained and Controlled Release Drug Delivery Systems, Marcel Dekker, Inc.
Seki, T., and Rutishauser, U. (1998). Removal of polysialic acid-neural cell adhesion molecule induces aberrant mossy fiber innervation and ectopic synaptogenesis in the hippocampus. The Journal of neuroscience: the official journal of the Society for Neuroscience 18, 3757-3766.
Stummeyer, K., Dickmanns, A., Muhlenhoff, M., Gerardy-Schahn, R., and Ficner, R. (2005). Crystal structure of the polysialic acid-degrading endosialidase of bacteriophage K1F. Nature structural & molecular biology 12, 90-96.
Schnaar, R. L., Gerardy-Schahn, R., and Hildebrandt, H. (2014). Sialic acids in the brain: gangliosides and polysialic acid in nervous system development, stability, disease, and regeneration. Physiol Rev 94, 461-518.
Senkov, O., Mironov, A., and Dityatev, A. (2015). A novel versatile hybrid infusion-multielectrode recording (HIME) system for acute drug delivery and multisite acquisition of neuronal activity in freely moving mice. Frontiers in neuroscience 9, 425.
Senkov, O., Sun, M., Weinhold, B., Gerardy-Schahn, R., Schachner, M., and Dityatev, A. (2006). Polysialylated neural cell adhesion molecule is involved in induction of long-term potentiation and memory acquisition and consolidation in a fear-conditioning paradigm. J Neurosci 26, 10888-109898.

The invention claimed is:

1. A polysialic acid according to the general formula (1) as given as follows and derivatives thereof:
(α(2→8)Neu5Ac)ₙ (1)
wherein Neu5Ac is N-acetylneuraminic acid, and
n is an integer in the range from 6 to 13,
for use in the prevention or treatment of a neurological and neuropsychiatric disorder,
wherein said derivatives are substituted with at least one sugar, acetyl group or acyl group at at least one monomer of the polysialic acid,
optionally linked to at least one nano-carrier,
and pharmaceutically acceptable salts thereof.

2. The polysialic acid of claim 1, wherein n is an integer in the range from 10 to 13.

3. The polysialic acid of claim 1 or 2, wherein the polysialic acid inhibits the activation of heterodimeric GluN1/GluN2B or heterotrimeric GluN1/GluN2A/GluN2B-containing NMDA receptors.

4. The polysialic acid of any one of the preceding claims, wherein said derivatives are substituted with at least one sugar, acetyl group or acyl group at one monomer of the polysialic acid.

5. The polysialic acid of any one of the preceding claims, wherein said derivatives are substituted with at least one sugar, acetyl group or acyl group at the one monomer at the non-reducing end of the polysialic acid.

6. The polysialic acid of any one of the preceding claims, wherein the at least one sugar is glucose, N-acetylglucosamine, N-acetylgalactosamine, galactose, fucose, mannose or xylose.

7. The polysialic acid of any one of the preceding claims, wherein said derivatives thereof have the formula (2) as given as follows: 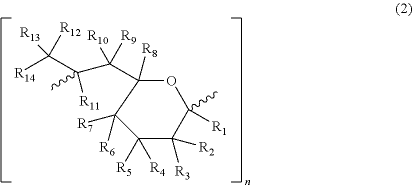
wherein at the one monomer at the non-reducing end of the polysialic acid:
i) R₁ is a carboxyl group;
ii) each of R₂, R₃, R₅, R₇, R₈, R₁₀, R₁₂, R₁₃ is independently from each other a hydrogen;
iii) R₄ is a hydroxyl group or an O-acetyl group;
iv) R₆ is NHCOCH₃;
v) R₉ is a hydroxyl group or an O-acetyl group;
vi) R₁₁ is a hydroxyl group or a hydrogen;
vii) R₁₄ is a hydroxyl group or an O-acetyl group.

8. The polysialic acid of claim 7, wherein at the one monomer at the non-reducing end of the polysialic acid:
i) R₄ is a hydroxyl group;
ii) R₆ is NHCOCH₃;
iii) R₉ is a hydroxyl group or an O-acetyl group;
iv) R₁₄ is an O-acetyl group.

9. The polysialic acid of claim 7 or 8, wherein n is an integer in the range from 10 to 13.

10. The polysialic acid of any one of the preceding claims, wherein the polysialic acid is chemically linked via its reducing end to a nano-carrier.

11. The polysialic acid of any one of the preceding claims, wherein the neurological and neuropsychiatric disorder is schizophrenia, tauopathy, bipolar disorder, depression, epilepsy, Huntington's disease, multiple sclerosis, amyotrophic lateral sclerosis or stroke.

12. The polysialic acid of claim 11, wherein tauopathy comprises Alzheimer's disease, frontotemporal dementia (FTD), primary age-related tauopathy (PART), chronic traumatic encephalopathy, progressive supranuclear palsy (PSP), corticobasal degeneration, dementia with Lewy Bodies, frontotemporal dementia and parkinsonism linked to chromosome 17 (FTDP-17), argyrophilic grain disease (AGD), glial globular tauopathy, Pick's diseases, and Parkinson's disease.

13. A pharmaceutical composition comprising as an active ingredient the polysialic acid and/or derivatives and/or pharmaceutically acceptable salts thereof according to any one of the preceding claims, optionally comprising a pharmaceutical acceptable carrier.

14. The pharmaceutically composition of claim 13, wherein the pharmaceutically composition comprises a pharmaceutically acceptable carrier, wherein the pharmaceutical acceptable carrier is a solid pharmaceutical acceptable carrier, preferably lactose, terra alba, sucrose, talc, gelatin, agar, pectin, acacia, magnesium stearate or stearic acid, a liquid pharmaceutical acceptable carrier, preferably sugar syrup, peanut oil, olive oil or water, or a gaseous pharmaceutical acceptable carrier, preferably carbon dioxide or nitrogen.

15. The pharmaceutical composition of claim 13 or 14, wherein the pharmaceutical composition is administered intranasal, oral, dermal, rectal, parenteral, preferably intranasal.

16. The pharmaceutical composition of claim 15, wherein parenteral administration comprises intravitreal injection, subcutaneous injection, intravenous injection, intraperitoneal injection, intramuscular injection, perfusion, infusion or topical administration.

17. The pharmaceutical composition of any one of claims 13 to 16, for use in the prevention or treatment of a neurological and neuropsychiatric disorder.

18. The pharmaceutical composition of claim 17, wherein the neurological and neuropsychiatric disorder is schizophrenia, tauopathy, bipolar disorder, depression, epilepsy, Huntington's disease, multiple sclerosis, amyotrophic lateral sclerosis, or stroke.

19. The pharmaceutical composition of claim 18, wherein tauopathy comprises Alzheimer's disease, frontotemporal dementia (FTD), primary age-related tauopathy (PART), chronic traumatic encephalopathy, progressive supranuclear palsy (PSP), corticobasal degeneration, frontotemporal dementia, dementia with Lewy Bodies, frontotemporal dementia and parkinsonism linked to chromosome 17 (FTDP-17), argyrophilic grain disease (AGD), glial globular tauopathy, Pick's diseases, and Parkinson's disease.

20. A method of producing the polysialic acid and/or derivatives and/or pharmaceutically acceptable salts thereof according to any one of claims 1-12, comprising:
a) dissolving colominic acid in acetic acid;
b) stopping the reaction of step a) with NaOH;
c) storing the mixture of step b);
d) separating oligo- and polymers by chromatography with a NaCl gradient;
e) obtaining the polysialic acid and/or derivatives and/or pharmaceutically acceptable salts thereof according to any one of claims 1-12.
